(19) United States
(12) Patent Application Publication
(10) Pub. No.: US 2023/0321817 A1
(43) Pub. Date: Oct. 12, 2023

(54) SOFT ROBOTIC TECHNOLOGIES, ARTIFICIAL MUSCLES, GRIPPERS AND METHODS OF MAKING THE SAME

(71) Applicant: NEWSOUTH INNOVATIONS PTY LIMITED, Sydney, New South Wales (AU)

(72) Inventors: Thanh Nho DO, Chester Hill, New South Wales (AU); Mai Thanh THAI, Chester Hill, New South Wales (AU); Phuoc Thien PHAN, Chester Hill, New South Wales (AU); Trung Thien HOANG, Chester Hill, New South Wales (AU); Nigel Hamilton LOVELL, Chester Hill, New South Wales (AU)

(21) Appl. No.: 18/042,383

(22) PCT Filed: Aug. 20, 2021
(86) PCT No.: PCT/AU2021/050924

§ 371 Date: Feb. 21, 2023

(30) Foreign Application Priority Data
Aug. 21, 2020 (AU) 2020902995

(51) Int. Cl. B25J 9/10; B25J 9/14; B25J 9/00; B25J 13/08; B25J 15/00; A61F 2/08; A61M 60/191

(52) U.S. Cl. CPC B25J 9/1075; B25J 9/142; B25J 9/0012; B25J 13/08; B25J 15/0009; A61F 2/08; A61M 60/191; B25J 9/0006

(57) ABSTRACT

An elongated actuator including: an elongated inner tube for carrying a pressurized actuation fluid; a helical coil wrapped around the elongated inner tube; wherein the actuator undergoes actuation by means of pressure fluctuations in the elongated inner tube.

### RELATED APPLICATION

The present disclosure claims benefit of priority to Australian Provisional Patent Application Number: 2020902995 filed 21 Aug. 2020, the contents of which are incorporated herein by reference. In jurisdictions where incorporation by reference is not permitted, the applicant reserves the right to add any or the whole of the contents of said Application Number: 2020902995 as an Appendix hereto, forming part of the specification.

### FIELD OF THE INVENTION

The disclosure relates to soft robotic technologies and specifically to soft artificial muscles and soft grippers for use in sensing and robotic technologies.

### BACKGROUND OF THE INVENTION

Soft robotic technologies offer the potential to transform the way humans interact with intelligent machines. Research interest in soft robotics, especially soft actuators, is increasing.

Actuation holds a fundamental position in every robotic system. Soft actuators belong to a novel branch of actuation mechanisms, in that they are made from soft, compliant materials and may exert motion and forces by outside excitation. In contrast to their rigid counterparts, soft actuators possess advanced properties such as flexibility, versatility, resilience to disturbances, adaptability to dynamic environments, and human-friendly interaction. The development of soft actuators follows two main streams involving the advancement of novel soft, active materials and structural design. The former approach has gained impressive results, leading to the presence of a wide range of amazing soft materials such as shape memory polymers (SMPs), liquid crystal polymers (LCPs), electroactive polymers (EAPs), hydrogels, liquid metals, ferrofluids, etc. On the other hand, the latter approach focuses on configurations, geometric arrangements of features or components of the soft actuators. The harmony of these two developing streams generates various mechanisms of operation and nurtures the development of fields such as artificial muscles, shape transformation sheets, soft grippers, soft robotic locomotion, and robotic fabrics.

There are soft muscles available in the literature that may produce elongation and force by various stimuli such as pneumatic (McKibben), electric (electroactive polymers, dielectric polymers), magnetic, and thermal.

External excitations play an essential role in soft robotic systems. They are not only a source of power but also a participatory element in the soft systems, significantly impacting the soft actuators' characteristics and performance There are four primary types of soft actuation based on their sources of excitation that have been widely studying and employing in the field of soft robotics.

1. Electric-driven actuation relies on the characteristic of response to electrical excitation of soft materials, for instance, electroactive polymers (EAPs). When receiving excitation, these materials will change their parameters, which generate motion and force. Due to the easy and versatile modulation of electric signals, this type of actuation has been well developed. Dielectric elastomer actuators (DEAs) was introduced by Pelrine et al. could achieve more than 100% of strain. The DEAs consist of a thin and compressible membrane located between two opposed electrodes. The electrostatic interaction of two electrodes deforms the membrane, generating actuation. The DEAs provide high-speed actuation, high strain, and the versatile performance by the customization of membrane material and thickness. However, the DEA requires a high operating voltage (several kilovolts), potentially causing electrical breakdown. The developers of Conductive polymers (CPs), won the Nobel Prize in Chemistry in 2000, are biocompatible and have low operating voltage. The CPs may produce moderate force but slow response time and require submersion in the electrolyte. Bay et al. reported a 50 mm strip of CPs could achieve 8% of strain in 20 s when receiving 1.5 V.

2. Magnetic-driven actuators are typically made from a combination of magnetic particles and soft, uncured silicone elastomers. Upon exposure to an external magnetic field (permanent magnet or electromagnet), the alignment movement of magnetic particles generates motion and force. Depending on the distribution and magnetization of these particles, which may be programmed at the fabrication stage, the various desired motions of the actuators may be achieved. The base materials would be soft, flexible polymers such as poly-dimethylsiloxane (PDMS), Ecoflex, and polyurethanes. Hu et al. published a small-scale soft robot that could perform multimodal locomotion by a programmable external magnetic field. The property of the magnetic attraction being wireless allows magnetic-driven actuators may be useful for many applications in confined spaces or require locomotion as microsurgery and drug delivery. Fast response time is another advancement point of this kind of actuation. However, the magnetic force will drop significantly when the distance between the actuators and the external source increase, and the operating of magnetic-driven actuation is greatly affected by the surrounding environment.

3. Temperature-driven actuation depends on the thermal conductive property of materials. The actuation is obtained by the deform of material structures under exposure to an external heat source like resistive heating, visible light, or infrared light. Jiang et al. introduced a bilayer robot (1×7 mm) made from PDMS and graphene nanoplatelets that could deflect 1.5 mm for multicycle by near-infrared irradiation within 3.4 s. Shape memory polymers (SMPs) in the work of Lendlein et al. could reach an elongation of up to 1000%. Temperature-driven actuators may be remotely activated so that they provide simple structural design and the ability to miniaturize the robot dimension. Although high strain, the inherent problem of these heat-activation actuators is notably slow response time, low energy efficiency, and low exertion force.

4. Pressure-driven actuators typically consist of a soft, deformable-body which has a chamber and a mean of pressure transmission and its container. When pressure is applied to the chamber, the actuators will generate motions respective to their structural design. The stiffer wall may resist the bending better than the softer one, resulting in the bending motion toward the stiffer layer. In other aspects, the actuators may elongate, expand, contract under pressurization. There are two main sources of pressure: air and fluid. Air is a lightweight and low viscous but may decrease the actuator response time due to its compressible attribute whereas fluid may exert high force with high frequency. Which source is being used relying on the specific applications. A popular actuator of this category is McKibben muscles with pneumatic actuation. Kurumaya et al. demonstrated a thin McKibben muscle could produce 15.77 N contraction force and 34% elongation under 0.5 MPa air pressure. Li et al. proposed a fluid-driven origami-inspired artificial muscle that produced 90% strain, 600 kPa stress, and 2 kW/kg power density.

Biology has long been a rich source of inspiration that guides the designs in the robotics field, especially the designs of soft robots. There are many instances in nature where continuum, helical manipulators are used to efficiently grasp objects of various shapes and sizes such as elephant trunks, snake or python body constriction, or cephalopod tentacles.

Over the years, more and more innovative applications in many areas such as human-machine interface and interaction, healthcare, haptics, locomotion and exploration, and assistive technologies have been enabled by the introduction of robots made out of intrinsically soft materials. Such robotic systems, while maintaining compliant structures, may inherently adapt to environments, conform to surfaces, exert forces, produce motions, and induce shape changes. Soft robotic devices that are wearable may interface with the human body for rehabilitation, movement assistance or virtual reality purposes. Soft robots in the form of end effectors may be used for tasks such as grasping, locomotion, surgery or even underwater operations. These soft robotic systems have emerged as a potential candidate to replace conventionally rigid counterparts in an attempt to create universal and versatile machines that possess capabilities to perform a wide range of tasks and active adaptability to changing conditions within those tasks.

Among all of the other applications, soft robotic grippers are one of the most extensively investigated research areas in the field of soft robotics. The inherent compliance of constituent materials enables soft grippers to safely work with flexible, fragile and delicate objects. A number of soft gripping technologies have been developed in the past 30 years and categorized into three groups, including actuation, controlled stiffness, and controlled adhesion. Gripping using controlled adhesion relies on surface forces at the interface between the gripper and objects for holding and typically requires another gripping actuation to partially envelope the object to be gripped. Controlled adhesion may be implemented via either electro-adhesion or dry adhesion, also known as gecko adhesion. Controlled adhesion is particularly suitable for manipulating very delicate objects because it eliminates the requirement of a large compressive force for grasping. This technology is also ideal for picking up flat objects which are difficult to be enveloped by the other two methods. However, controlled adhesion has a limitation that it requires clean, dry and relatively smooth surfaces to be effective. Controlled stiffness, on the other hand, exploits the significant variation in stiffness of some materials when they transform between rigid and soft states. As the gripper in the soft state, it may grasp and envelope delicate objects with a lower force while being in the rigid state, the gripper may have higher holding force. In a similar manner as controlled adhesion, controlled stiffness is usually used in combination with another actuation mechanism to grip the object (except granular jamming that may be used directly to grip objects). When being combined with other gripping actuation, controlled stiffness may also be known as variable stiffness structures. Two main types of variable stiffness mechanisms include phase-change materials such as thermoplastics, shape memory polymers (SMPs) or low-melting-point alloys (LMPAs) and vacuum-driven jamming of granules or layers. Stiffness of these structures may vary over a broad scale, ranging from a few MPa to a few GPa. Gripping by actuation is the final and the largest group among the three categories. In this method, gripper fingers or elements are bent to wrap around the object to be gripped. The bending shape may be either actively controlled or passively induced by the contact with the object due to the compliance of gripper materials. A great number of actuation methods have been investigated for gripping by actuation, including cable-driven, fluid elastomer actuators (FEA), dielectric elastomer actuators, and shape memory materials including alloys and polymers. Among these actuation methods, FEA is one of the oldest and the most widespread technologies employed for soft robotic grippers owing to a number of advantages, including lightweight, high power-to-weight ratio, large stroke and force production, easy fabrication, robustness and low-cost materials.

FEA-based soft grippers have been mostly developed based on claws or human-like structures consisting of multiple inward-bending fingers. This configuration is suitable for gripping objects spanning a wide range of sizes; however, its conformability is not good for objects with irregular shapes, and its load capacity is also limited due to the mechanical compliance of constituent materials. On the one hand, the latter issue may be resolved by the combination of these grippers with variable stiffness structures. Meanwhile, there have been studies conducted to address both the aforementioned issues by designing fingers that may bend with adjustable lengths via the use of separately embedded variable stiffness segments. On the other hand, grippers with closed structures have been investigated in an attempt to improve both conformability and load capacity. Brown et al. and Amend et al. demonstrated universal grippers with closed structures based on granular jamming that could hold a load up to 8.5 kg and reliably grasp objects of different shapes and sizes. Li et al. reported vacuum-actuated grippers made of flexible thin membrane and origami shell that were lightweight and highly conformable. However, grippers with closed structures are restricted on the size range of objects that they may grip, which means they cannot grip objects that are larger than the opening orifice of the gripper.

Another approach that has been investigated involves grippers that are designed to enclose objects via helical winding. This gripping strategy got inspiration from natural instances such as elephant trunks, python body constriction or cephalopod tentacles that use a continuum finger to helically grasp around the objects, thereby increasing the area of contact and stability between the gripper and objects. Continuum, helical grippers that are not constrained by any host have the advantage of being free to wrap around objects and adapt to a large variety of object sizes, shapes and orientations. Especially, these grippers may be particularly suitable for gripping long and slender objects that have been challenging for single-point gripping of conventional finger-based designs. The continuum construction with a small footprint of helical grippers also makes them ideal for retrieving objects from confined spaces such as long, narrow tubes that grippers with finger designs normally cannot reach or for hooking through holes or slots available on object bodies, providing an alternative way to grip objects that are challenging to wrap around. There have been several studies focusing on this approach over the past few years. For example, Martinez et al. introduced a single, soft tentacle able to hold flowers and wrap around and lift a metallic wrench. Uppalapati et al. and Bishop-Moser et al. reported the use of pneumatic Fiber Reinforced Elastomeric Enclosure (FREE) actuators to develop helical manipulators that could grip long and slender objects such as light tubes and PVC pipes. Galloway et al. presented the development of a helical fiber-reinforced gripper for underwater retrieval of specimens from the deep reef. More recently, Guan et al. contributed to this gripping strategy with the work on the development of helical extensile/contractile actuators based on Pneumatic Artificial Muscles (PAMs). The fabricated prototype was able to grip objects of different shapes and sizes, such as a water bottle, a glue gun and a digital multimeter. Nevertheless, similar to other FEA grippers, these continuum, helical grippers generally exhibit low load capacity due to inherent characteristics of soft materials. Li et al. reported the development of high-load soft grippers with PAM actuators arranged in helical winding patterns. The reported gripper could lift heavy objects that weight up to 35.5 kg, which is 47 times of the gripper weight. However, these grippers were fabricated following the closed gripping structure, which restricts their object sizes. As a result, it will be beneficial to have a gripper that possesses advantages of both designs, being continuum to fit a wide range of object sizes and strong enough to handle large loads.

In addition to the development of gripping methods, soft sensing technologies have been extensively studied to improve the performance of soft robotic grippers. These sensors may be either proprioceptive, which monitor the status of gripper elements such as curvature or bending angle or exteroceptive, which detect external stimuli like contact pressure and gather information about the objects. The working principle of these sensors may be based on resistive, capacitive, optical waveguide or magnetic sensing. Among these technologies, resistive sensing is the most widely applied technology because of its facile fabrication and readout scheme. Soft elastomers with prefabricated microchannels injected with liquid metals such as eutectic gallium-indium (EGaIn) have been favorably used as stretchable, resistive sensors in soft robotics field recently because of its high stretchability, reliability, high electrical conductivity and low melting temperature (beneficial for fabrication). Many studies have reported the use of soft, resistive sensors filled with liquid metals for both single and multi-modal sensory feedback in soft grippers. However, so far, liquid-metal-based soft sensors have been mostly fabricated by injecting liquid metals into microchannels prefabricated in blocks of soft elastomers. This fabrication process requires multiple steps of molding and bonding to create microchannels, which causes the workflow complicated and labor-intensive. Meanwhile, microchannels of liquid metal embedded inside an elastomer block usually have reduced sensitivity due to limited strain that may be induced under stress compared to liquid metal microtubules arranged in the same pattern but not having as much elastomer material surrounding.

Any discussion of the background art throughout the specification should in no way be considered as an admission that such art is widely known or forms part of common general knowledge in the field.

### SUMMARY OF THE INVENTION

In accordance with a first aspect the present invention, there is disclosed a soft hydraulic filament artificial muscle comprising an inner microtubule and an outer coil and configured to extend and contract in response to differentiation in hydraulic pressure.

The soft hydraulic filament artificial muscle (HFAM) overcomes many challenges of existing actuation technologies. The HFAM in some forms can operate at low nonlinear hysteresis, long-distance, and high energy efficiency, overcoming major limitations of high friction loss, low force transmission from expensive tendon actuators.

The soft muscle may be valuable in applications involving shape programmable robots, smart textile/fabric, assistive wearable systems, soft wearable haptic, shape-shifting structures, bio-inspired soft robots or creating active robotic skin that may turn passive conformable sheets into active elements.

In some forms, disclosed is the use of HFAM to form complex programmable robots and structures to develop compression devices for heart failure, exo-suits, soft grippers, and others. The soft actuator may be twisted, braided, knitted, and weaved to create smart garments or integrated into fabrics to reconfigured passive objects to become active ones.

Also disclosed is a method of fabricating a hydraulic filament artificial muscle, the method comprising inserting a microtubule into a hollow helical coil. In some forms, the method further comprises closing the first end of the microtubule and adhering it to the coil. In some forms, the method further comprises attaching a second end of the microtubule to a fluid source.

The fabrication method may, in some forms, have the benefit of obtaining a high aspect ratio of HFAM ranging from several micrometers to millimeters scale. The fabrication method to obtain HFAM is conceptually and methodologically innovative because it overcomes existing challenges for creating miniature, high aspect ratio microtubule with arbitrary shapes and at scales. The fabrication method may have economic significance because the HFAM may be mass-produced at low cost while overcoming the low energy efficiency, low adaption compared to existing technologies. The novel fabrication methods may be used for both academic research and commercial purposes. The new fabrication method in some forms avoids the complexity of manual wrapping of fiber along a hollow silicone body that requires strict uniform distribution of helical structure in order to avoid ballooning during the operation as well as overcoming limitations on the muscle lengths.

In some forms, disclosed are assistive compressive medical devices based on the innovative HFAM, which may restore the function of human organs with diseases such as heart or bladder failure. The flexibility and versatility of HFAM may, in some forms, permit a conformability to the complex tissue surface, enabling high effectiveness to support the organ as it may mimic the complex motion of these organs in a biomimetic fashion. It will overcome major limitations from existing devices and technologies to support a large number of patients.

Further, disclosed in some embodiments is a novel HFAM-based exoskeleton suit to augment the human performance on lifting or carrying heavy objects in defence, military, space, industry, or to support disabled patients in rehabilitation. It allows assistive devices to provide mechanical support to both lower and upper limbs or the whole human body.

In some forms, disclosed are smart garments such as adjustable clothes, shoes, or gloves to provide a customized fit and effective protection. It may also be used to make compression sleeves for arms, legs, or body to serve as massage therapy to relieve pain or improve blood circulation. These devices have a large market ranging from hospitals, general practitioners, and individuals as sportsmen, workers, or the elderly.

In some forms, disclosed are configurations of HFAM to create multifunctional devices and shape-shifting robots that give significance to medicine and rescue tasks such as bushfires, natural and man-made disasters, collapsed buildings. The new technologies will also advance knowledge in the realm of physically programmed robots, adding new understanding about the scalability of such systems to specific environments, exploring optimal control, and shape change of a given topology.

Disclosed in some forms is a bio-inspired soft gripper that makes use of the continuum, helical configuration, to achieve highly conformable grasping of objects of various shapes and sizes. The small footprint of the proposed gripper is proved to be useful for retrieving objects in confined environments such as pipelines and for gripping by hooking through holes/slots when objects are challenging to wrap around. The helical gripper is incorporated with a variable stiffness structure made of thermoplastic material to enhance the load capacity. A highly sensitive contact sensor with novel design, employing stretchable, liquid-metal microtubules, is also integrated into the gripper, providing the sensory feedback of contact pressure when the gripper finger touches the objects.

Disclosed, in some forms, is a new gripper design that combines both the flexibility of fingered grippers and high load capacity due to the variable stiffness structure. In addition to the single-channel design, the continuum, the helical gripper may be fabricated to suit applications at multiple scales, depending on the size and load capacity requirements. Because silicone tubes, inextensible coils and fabric sleeve may be fabricated at different scales (diameter, length, number of channels), the gripper may have plenty of sizes, ranging from millimetres to centimetres that may fit tiny spaces or lift much heavier objects. The materials constituting the grippers are abundantly available in the market while their fabrication process employs reliable computerized methods of apparel engineering, which makes them suitable for mass production. Thanks to the simple design and facile fabrication process that eliminates complex molding structures, it is also possible to quickly modify the gripper surfaces, both top and bottom, with a variety of textures to expand the task versatility. For example, a thin silicone layer with microstructures, mimicking the gecko adhesion, may be attached to the bottom surface of the gripper to increase the frictional force, and thereby, the load capacity of the gripper. For applications involving holding ultra-delicate samples, other materials such as soft foam may also be used to maximize the protection of samples from damaging.

Disclosed, in some forms, is a helical soft fabric gripper with a variable stiffness structure for high load capacity and a stretchable, liquid-metal-based sensor for touch sensing. The continuum gripper was constructed from a hydraulic-pressurized silicone tube constrained by an inextensible coil and an anisotropic fabric sleeve. The variable stiffness structure made from PET tubes enabled the gripper to successfully lift a 1.8 kg object, which is 220 times the mass of the gripper. An active cooling system employing a vortex tube could significantly increase the cooling rate, allowing the variable stiffness structure to complete thermal cycles within 24 s. The novel design of the stretchable, liquid-metal-based sensor produced a remarkably enhanced sensitivity, i.e., a 157% change in sensor resistance at 15 kPa normal contact pressure and 15 times higher than the value of conventional designs. The gripper successfully exhibited the ability to grasp and lift objects of a wide range of shapes and weights. In addition, the thin and flat geometry of the gripper was shown to be ideal for object retrieval from confined spaces. The design of the proposed gripper is also scalable and may be easily modified to suit applications of different requirements. The helical soft fabric gripper may be of great potential for applications in areas such as gripping fragile objects or objects of arbitrary shapes and various weights, exploration, rescuing, maintenance or manipulation applications in confined and dangerous environments such as gas/oil or drainage industry.

In accordance with another aspect of the present invention, there is provided an elongated actuator including: an elongated inner tube for carrying a pressurized actuation fluid; a helical coil wrapped around the elongated inner tube; wherein the actuator undergoes actuation by means of pressure fluctuations in the elongated inner tube.

In some embodiments, the inner tube is open at at least one end and attached to a fluid pressure control means for causing controlled pressure fluctuations in the inner tube. In some embodiments, the helical coil formed from one of metal wire, fishing line, a polymer or sowing thread. In some embodiments, the actuator is twisted, knitted, weaved, or braided to form a fabrics or rope structure.

In some embodiments, a collection of elongated actuators is attached to at least one substrate so as to cause relative controlled movement thereto. In some embodiments, actuator tube expands on pressure increase and contracts on pressure decrease. In some embodiments, a wide range of controlled motions can result, including contraction, elongation, bending, shape-shifting structure.

In some embodiments, the actuator is woven into a fabric suitable for use in a heart assist device, or into a fabric suitable for use in a muscle assist device. In some embodiments, the actuator forms a helical gripper. In some embodiments the fluid is actively cooled. In some embodiments, a surgical suture is formed from an actuator.

### BRIEF DESCRIPTION OF THE DRAWINGS

Embodiments of the invention will now be described, by way of example only, with reference to the accompanying drawings in which:

FIG. 1 illustrates schematically a muscle system;

FIG. 2 illustrates the corresponding one embodiment of the soft hydraulic filament artificial muscle (HFAM);

FIG. 3 shows application areas of the bioinspired soft hydraulic filament artificial muscles;

FIG. 4 shows a fabrication process for one embodiment of the soft hydraulic filament artificial muscle (HFAM) and its special arrangement to form different planar structures or to tune passive objects to active ones;

FIG. 5 shows two operating types of HFAM. A) Single HFAM without sheath; B) HFAM with routing sheath;

FIG. 6 illustrates schematically a generated force and motion diagram of HFAM with respect to the applied input pressure;

FIG. 7 shows several HFAM prototypes.

FIG. 8 to FIG. 11 shows elongation and force responses of a single HFAM with respect to the change of applied input volume and pressure. A, B) Hysteresis profile which shows the relation between input volume, input pressure and output elongation in both forward and backward directions; C, D) Hysteresis profile which shows the relation between input volume, input pressure and output force in both forward and backward directions.

FIG. 12 shows variants of the HFAM for the comparison tests.

FIG. 13 to FIG. 16 shows performance comparison between five variants of the HFAM in terms of elongation and force. Force data were collected at the initial elongation of approximately 87% for all variants.

FIG. 17 and FIG. 18 illustrate the maximum elongation of a single HFAM.

FIG. 19 to FIG. 21 shows lifting performance A. HFAM1 (length 50 mm) lifts a weight of 100 grams. B. HFAM2 (length 50 mm) lifts a weight of 500 grams. C. One-dimension weaving sheet lifts a weight of 500 grams.

FIG. 22 shows a compression garment for a finger. Merge two ends of the sheet (A) to form a cylindrical sleeve (B), then wear it in an index finger (C). D. E. Side views of the sleeve when releasing and pressurizing, respectively. The sheet is made from a 400 mm long HFAM and acrylic yarns with the original dimension of 57×27 mm.

FIG. 23 shows the shape transformation of a two-dimension weaving sheet.

FIG. 24 shows bio-inspired soft robots. A. The principle structure of embedding and routing HFAM in fabric layers by double-sided tape. B-D. 4-legged robot at the initial phase, medium pressurization, and high pressurization, respectively. E. Butterfly robot and flower robot with embedded HFAMs under the wings and petals. F-H. The flower robot at the initial phase, pressurizing only the top-layer petals, and pressurizing both layers of petals, respectively. I-K. The butterfly robot at the initial phase, medium pressurization, and high pressurization, respectively.

FIG. 25 shows artificial muscles. A. A pair of HFAM1, each has the original length of 600 mm, quadruple twisted configuration, the initial length of 135 mm B C The artificial muscles mimic the human bicep and tricep. D. A pair of HFAM2, each has the original length of 90 mm, single configuration, coated with a layer of EcoFlex. E. F. The artificial muscles mimic the main muscles of the human index finger;

FIG. 26 shows knitting and weaving HFAM. A. Knitting sheet made from two single HFAMs and elastic strings at the initial phase (i); pressurizing the muscle 1 (ii); pressurizing the muscle 2 (iii); pressurizing both muscles (iv);

FIG. 27 illustrates B. One-dimension weaving sheet made from a single HFAM and acrylic yarns at the initial phase (i) and pressurizing phase (ii);

FIG. 28 illustrates a two-dimension weaving sheet made from two single HFAMs and acrylic yarns at the initial phase (i) and pressurizing phase of both muscles (ii);

FIG. 29 shows radial expansion. A. Spiral configuration of a single HFAM generates planar radial expansion;

FIG. 30 illustrates B. Helical configuration of a single HFAM generates cylindrical radial expansion. Both configurations are secured by acrylic yarns by weaving technique;

FIG. 31 illustrates the design of the compression robotic device (CRD);

FIG. 32 shows the compression robotic device (CRD) working principle;

FIG. 33 illustrates one form of incorporation of a CRD device;

FIG. 34 illustrates an alternative form of CRD device;

FIG. 35 shows the fabrication of one form of CRD device;

FIG. 36 shows one form of CRD deployment;

FIG. 37 shows one form of the CRD that wraps around a porcine heart;

FIG. 38 shows an alternative form of the CRD device;

FIG. 39 shows an exo-suit made by embedding HFAMs into garments;

FIG. 40 illustrates one form of HFAM glove;

FIG. 41 illustrates the utilisation of HFAM in an outer garment;

FIG. 42 illustrates photographs of a prototype outer garment;

FIG. 43 shows (a) The time history of the proposed model, the experimental elongation in the HFAM and the error result between them;

FIG. 44 illustrates the nonlinear hysteresis predicted by the proposed model for 0.1 Hz input signal;

FIG. 45 illustrates the time history of the proposed model, the experimental elongation in the HFAM and the error result between them;

FIG. 46 illustrates the identified results for multi-periodic input combined by two sinusoidal signals of frequencies of 0.1 Hz and 0.2 Hz;

FIG. 47 shows the time history of the proposed model, the experimental elongation in the HFAM and the error result between them;

FIG. 48 illustrates the nonlinear hysteresis predicted by the proposed model for a non-harmonic sequence of the input signal with frequencies of 0.1 Hz and 0.1\sqrt{3};

FIG. 49 illustrates the time history for 0.1 Hz input signal, the time history and nonlinear hysteresis for 0.15 Hz input signal, and 0.2 Hz input signal;

FIG. 50 illustrates the nonlinear hysteresis for the signals of FIG. 49;

FIG. 51 illustrates schematically a proposed bio-inspired soft, helical gripper with potential uses in gripping fragile objects, conforming to different shapes, lifting heavy objects and working in confined spaces.

FIG. 52 shows a schematic illustration of the design of soft, helical gripper incorporated with a variable stiffness structure and soft, liquid-metal-based contact sensor.

FIG. 53 illustrates an exploded perspective of the helical gripper;

FIG. 54 shows a schematic of the fabrication process for the twisting core actuator of the soft gripper;

FIG. 55 shows fabric and stitch selection for the fabric sleeve;

FIG. 56 shows a schematic of the fabrication process for the fabric sleeve;

FIG. 57 illustrates an example fabrication of a fabric sleeve;

FIG. 58 shows the variable stiffness structure made of PET tube and heating coil. Top: demonstration of phase transition of the VST between glassy and rubbery states;

FIG. 59 illustrates the working principle of the vortex tube that is used to shorten the cooling process;

FIG. 60 shows a first design for soft, stretchable resistive sensors based on liquid metals;

FIG. 61 shows a second design, more conventional design;

FIG. 62 shows a structure of the core actuator with deformation parameters. A) Helix angle α of the inextensible coil and rotation parameter ω. B) Diameter and radii of the actuator under low and high pressures. C) The actuator is undergoing deformation with a helix radius of curvature ρ.

FIG. 63 shows motions of the soft, helical gripper corresponding to different input pressure values.

FIG. 64 shows an experimental setup for characterization of contact sensors, for the measurements of resistance as a function of normal contact pressure;

FIG. 65 illustrates the schematic diagram of the readout circuit of FIG. 64;

FIG. 66 illustrates the comparisons of the change in resistance, ΔR/R, as a function of normal contact pressure;

FIG. 67 is a photo of the two sensor designs;

FIG. 68 illustrates a wavy-shaped sensor integrated on the helical gripper;

FIG. 69 illustrates the dynamic response of a Helical gripper grasped with no load;

FIG. 70 illustrates the dynamic response for a helical gripper grasping a screwdriver handle;

FIG. 71 shows experimental setups for characterization of holding force, including a schematic explaining the experiment setup;

FIG. 72 illustrates a photo of an arrangement of FIG. 71;

FIG. 73 shows the results of pulling experiments, including a comparison of peak holding forces between gripper with and without VST in horizontal and vertical pulling tests. The error bar represents three standard deviations (3σ).

FIG. 74 shows an embodiment of a helical gripper grasping objects with multiple shapes and sizes. (A) Left to right: cylinder, cone, rectangular prism and gourd;

FIG. 75 shows a Lateral view of helical gripper grasping four objects;

FIG. 76 illustrates a top view of helical gripper grasping four objects;

FIG. 77 shows examples of the helical gripper gripping multiple objects. (A) Syringe. (B) Marker. (C) Cylinder container with drill bits. (D) Plastic tripod. (E) Screwdriver. (F) Superglue bottle. (G) Hand saw. (H) Hammer (I) Lemongrass. (J) Cucumber. (K) Grape. (L) A long tube of 540 mm length.

FIG. 78 shows examples of helical gripping in confined space: (A) Marker. (B) Screwdriver. (C) Metallic wrench;

FIG. 79 illustrates examples of gripping by hooking through the center holes of a masking tape roll. Examples of using VST to lift heavy objects: (E) Zehntner film applicator case. (G) Bosch tool case.

FIG. 80 illustrates a smart surgical suture, with typical stitches;

FIG. 81 illustrates potential application areas of a typical stitch;

FIG. 82 illustrates various forms of the smart surgical suture;

FIG. 83 to FIG. 85 illustrate various forms of a pressure locking mechanism for the suture;

FIG. 86 to FIG. 88 illustrate photos of knot self-tightening ability of the sutures;

FIG. 89 illustrates the use of the sutures in perforation closure;

FIG. 90 illustrates the use of the sutures in tissue folding;

FIGS. 91 and 92 illustrate the use of the sutures in perforation closure;

FIG. 93 illustrates the use of the sutures in cerclage correction; and

FIG. 94 illustrates a prototype suture in operation.

### DETAILED DESCRIPTION

Disclosed in some forms is a soft filament artificial muscle comprising an inner microtubule and an outer coil, the muscle being configured to extend under hydraulic pressure. In some forms, the muscle is configured to retract at a reduction in hydraulic pressure.

Inspired by these natural examples, a continuum, helical soft fabric gripper that is thin, lightweight, and scalable is disclosed. The gripper was hydraulic-driven and could be fabricated by a facile fabrication process without complicated steps of molding. The construction of the gripper also incorporated a thermally activated variable stiffness structure (VST) for high load capacity that may complete a softening-stiffening cycle within 24 s, which is among the fastest results reported so far. In addition, a stretchable, liquid-metal-based sensor with a novel design for enhanced sensitivity (15 times more sensitive compared to conventional designs) was added to the gripper for the touch sensing purpose. The continuum, helical gripper was proved to be applicable in multiple scenarios, including gripping fragile objects, grasping objects of different geometries and weights (up to 220 times the mass of the gripper itself), and retrieving objects from confined spaces such as pipelines. This property makes the gripper ideal for exploration, rescuing, and manipulation applications in confined and hazardous environments such as gas/oil or drainage industry.

In some forms, the inner microtubule is configured to engage a fluid source at a fluid engagement end such that fluid pressure in the microtubule may be varied.

Disclosed, in some forms, is a method of fabricating a soft filament artificial muscle comprising inserting a microtubule into an outer coil, the microtubule having first and second ends. In some forms, the method comprises tying off the first end of the microtubule and engaging it with the coil. In some forms, the method comprises attaching the second end of the microtubule to a fluid source.

Also disclosed, in some forms, is a soft filament artificial muscle being incorporated into shape programmable robots, smart textile/fabric, assistive wearable systems, soft wearable haptic, shape-shifting structure, a bio-inspired soft robot, an active robotic skin, assistive compressive medical devices or HFAM-based exoskeleton suit to augment the human performance on lifting or carrying heavy objects among other systems.

In some forms, disclosed is a soft filament artificial muscle being configured to be twisted, knitted, or weaved to create smart garments or reconfigure passive objects to become active ones.

In a further embodiment, disclosed is a gripper system comprising a soft fabric gripper having a continuum helical shape. In some forms, the soft fabric gripper comprises a core actuator that is hydraulic-driven. In some forms, the soft fabric gripper comprising a fabric sleeve 43 that constrains and causes the core actuator to bend. In some forms, the gripper system comprises a contact sensor. In some forms, the gripper system comprises a variable stiffness structure to enhance the load capacity.

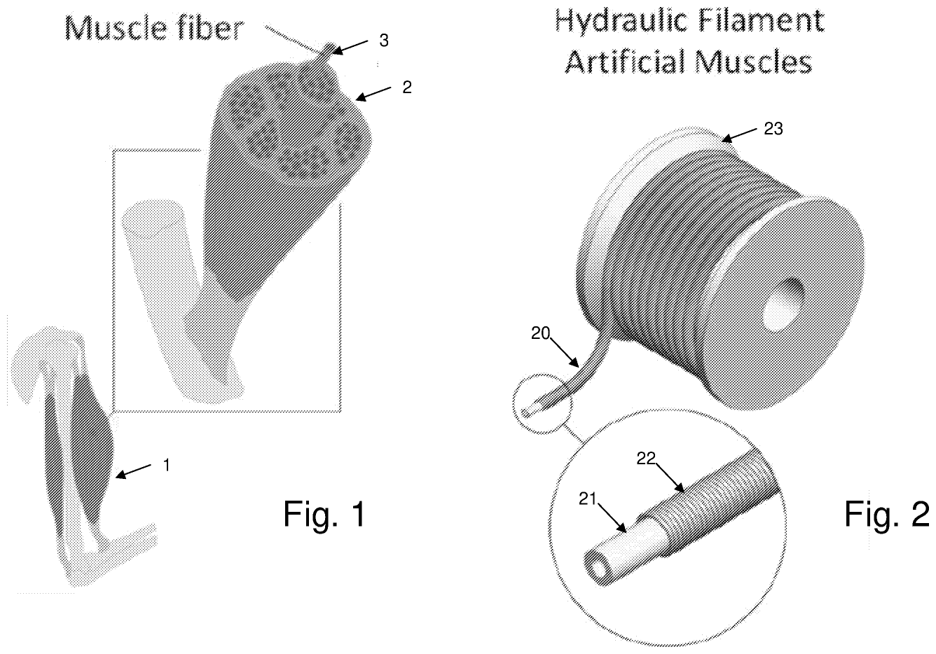

Referring initially to FIG. 1, there is disclosed a schematic illustration of an arm muscle structure 1, shown in an enlarged sectional form 2, with a muscle fiber 3. The embodiment fiber is designed to replace such fiber with an analogous use. There are multiple application areas for artificial muscles in the form of soft microtubule muscles. These include wearable suits, compression garments and robotic fabrics.

Referring to FIG. 2, there is illustrated a roll of the soft microtubule muscle 20 which consists of an inner microtubule 21 and an outer helical coil 22, both of which may be customized in terms of size and material. The HFAM 20 is configured to extend lengthwise under increased hydraulic pressure and to contract when the pressure is released. Fluid powers the HFAM, which has benefits as fluid is an incompressible means with low hysteresis and high response time. However, pneumatic is also an example a source of actuation.

Fluid provides the characteristic of high force transmission, fast response, and high energy efficiency. The actuator disclosed can be scalable and multifunctional soft actuator that may produce high elongation.

In some forms, the simple yet effective manufacturing method enables mass-production of a long HFAM, and the ability to reserve it in a spool for mass demands A single HFAM may reach a maximum elongation of 246.8%, lift a weight which is 352 times heavier than its mass, and achieve 62.7% of energy efficiency. A single HFAM may utilize twisting, braiding, knitting, and weaving with various configurations to enhance the capability of the HFAM, augmenting its functions as in artificial muscles, robotic fabrics, compression garments, and shape transformation.

Multifunctional, soft, hydraulic filament artificial muscle (HFAM) is a long, high aspect ratio, miniature actuator that may be twisted, knitted, weaved, braided to form smart textiles/fabrics at scale or flexibly embedded into arbitrary passive objects to induce desired motions and deformations. These configurations may scale up the force, amplify the motion or increase the number of degrees of freedom (DOFs) of its planar structure by a parallel assembly of single HFAM that is lengthened or contracted under the applied fluid pressure.

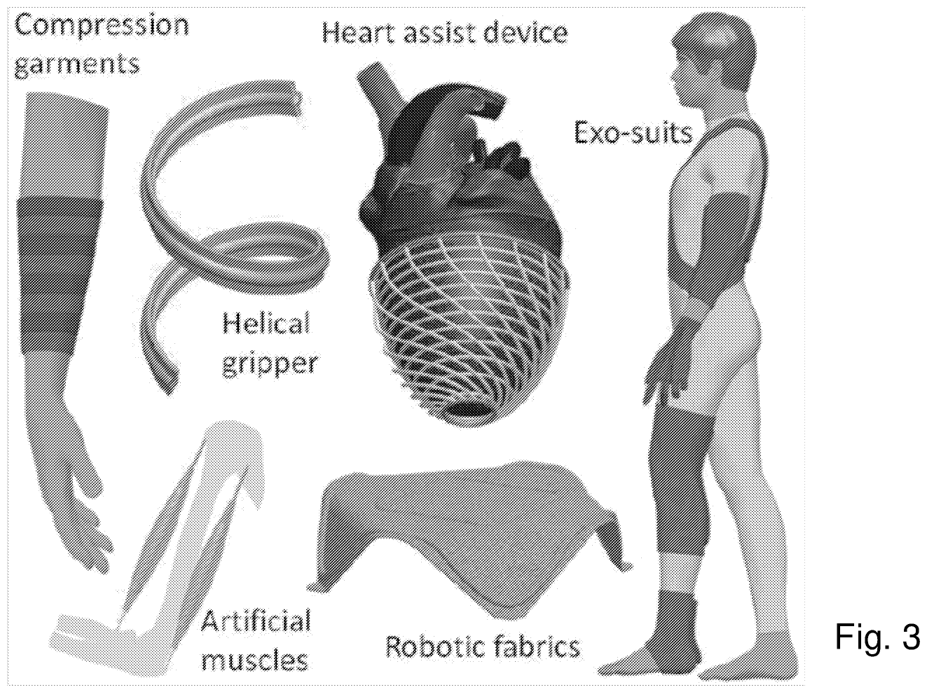

Turning to FIG. 3, there is shown various example uses for the HFAM, which will be discussed further herein after, including compression garments, helical grippers, artificial muscles, heart assist devices, robotic fabrics and exo-suits.

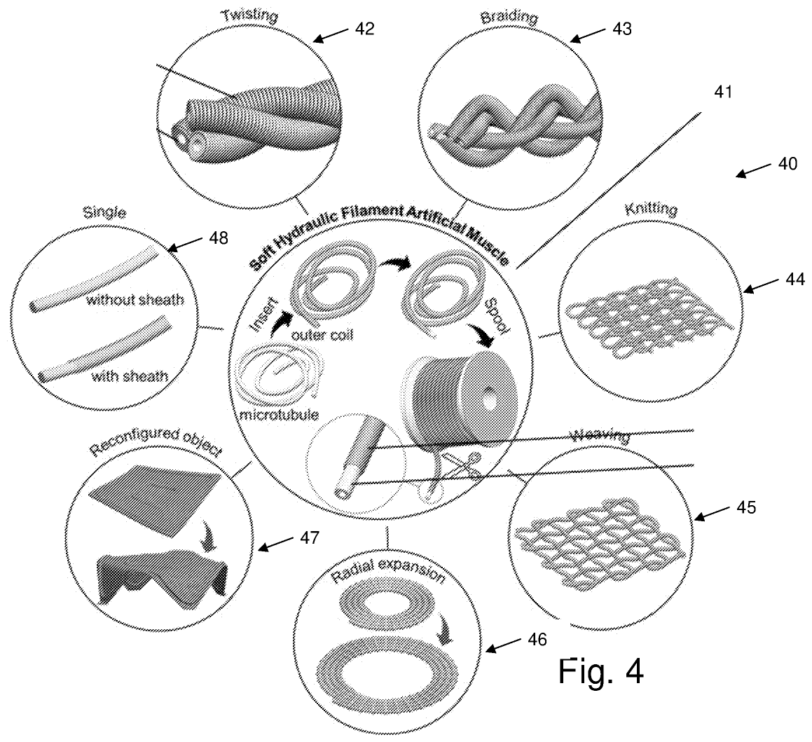

As further shown in FIG. 4, different configurations of a high aspect ratio hydraulic filament muscle, when pressurized hydraulically, generate a wide range of motions. These motions were changed in both amplitudes and directions simply by varying the arrangement of the filament muscles in their associated structure of conformable planar sheets through twisting, knitting, weaving, braiding or directly laminate onto the surface of 3D objects to turn their status from passive to active motions.

As shown 40 in FIG. 4, the HFAM can be subject to various uses. Including twisted together 42, braiding 43, knitting 44, weaving 45, radial expansion 46, reconfigurable objects 47, single use 48.

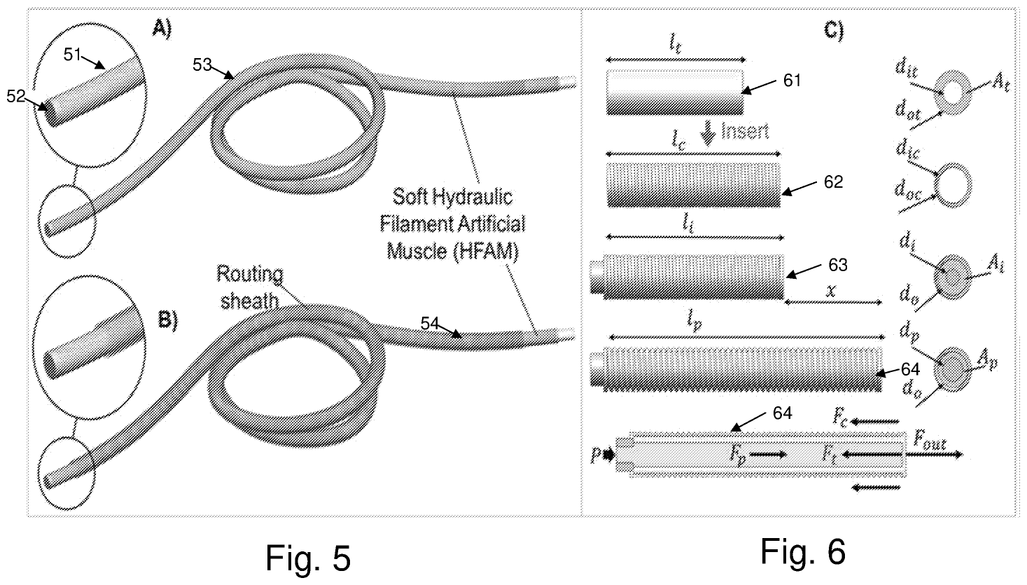

FIG. 5 illustrates generally the HFAM structure.

As shown in FIG. 6, to obtain the HFAM, a soft microtubule 61 is directly inserted into a hollow helical coil 62 to form the HFAM structure 63. In some forms, insertion is performed using a sewing thread or fishing line as a guider, which is attached to a micro-needle or a long micro-carbon fiber rod. Once the soft microtubule is completely inserted into the micro-coil, its one end may be tied into a knot or otherwise closed off and maybe permanently or semi-permanently adhered onto the micro-coil end by, for example, an adhesive glue (LOCTITE®, USA) while the other end may be connected to a fluid tube which is connected to fluid control source.

Unlike some conventional approaches on fiber-reinforced soft extensible actuators where inextensible fiber is manually wrapped around a hollow soft elastic tube, in some forms of the disclosure, the fabrication leverages the simplicity of insertion method, in which an inner elastic microtubule is directly routed through an outer constrained helical coil without directly wrapping constrained fiber along the soft microtubule body, allowing facile manufacturing of miniature, meter-long muscle with the diameter ranging from few hundred micrometers to several millimeters.

In some forms, the disclosed fabrication method may avoid the strict requirement of uniform fiber wrapping along the soft elastic microtubule, which poses many challenges for any fluid-driven soft actuators at micrometer scale and high aspect ratio. The inner soft microtubule 61 from the disclosed HFAM may be made from a wide range of materials such as silicone rubbers, Ecoflex™ series (Smooth-On, Inc., Macungie, PA, USA) and other silicone elastomers such as NuSil™ (NuSil™ Technology LLC, Carpinteria, CA, USA) and may use previous methods of rolling coating process or off-the-shelf silicone microtube which are well suited with extrusion or dip coating processes such as ones available from Saint-Gobain S. A., Courbevoie, France, and McMaster-Carr Supply Co., Elmhurst, IL, USA. One of the advantages of this approach may be the use of a separate constrained outer layer which is a type of long helical coil that may be manufactured from a diversity of inextensible materials such as stainless steel wires, brass wires, fishing line or sewing thread. A winding machine that provides rotational motion of a mandrel and longitudinal translation of a wire guide simultaneously may be used to produce the constrained outer layer. Alternatively, this constrained helical coil may be off-the-shelf components such as ones available from McMaster-Carr Supply Co., USA.

HFAM may have the ability to receive power sources from a variety means of transmission, including compressible air or incompressible fluids such as water, saline, or oil. In the disclosed design, the long HFAM is stored in a spool that may be cut at a predefined length for specific applications or alternatively be made excessively long. In one example, a meter long, miniature HFAM was fabricated from stainless steel coil as an outer constrained layer and soft silicone microtubule with an outer diameter of 0.84 mm and a length of 4 m (OD0.84 mm×L4000 mm). The obtained HFAM has a great aspect ratio of 4762:1 that is extremely high compared to existing hydraulic or pneumatic soft muscles available in the literature.

As shown in FIG. 6, one of the main advantages of HFAM may be the capability to transmit mechanical force or energy from a distance, similar to that of the conventional flexible tendon or cable mechanisms in which its one end is fixed and the other end moves.

As shown in FIG. 5, HFAM may exist in two main types, including HFAM with sheath 54, where the movement of an inner HFAM relative to a hollow outer sheath and HFAM without sheath 53 where HFAM directly conveys force and motion without using any routing supports. Compared to tendon or cable systems, both types of HFAM offer higher energy efficiency due to the use of fluid source, allowing its external power supply is located at a long distance without scarifying its input energy. In addition, HFAM generates its motion via local extension of individual muscle segment, which is analogous to the natural behavior of certain plant cells that are lengthened or shortened when being pressurized, enabling uniform distributions of the motion and its generated mechanical force while maintaining its energy regardless of the distance. In contrast, the motion and force output of the tendon or cable mechanisms highly depends on the distance of its power source located at its proximal end.

In applications that require highly tortuous path such as flexible surgical robots or wearable exoskeleton where tendon-sheath or Bowden cable mechanism are currently dominant, most energy loss originates from the nonlinear friction between the tendon and guided path. HFAM, in contrast in some forms, may avoid this limitation by local sliding of the muscle element over the inner sheath surface, allowing minimal energy loss. Without helical constraint, the inner soft microtubule may be expanded axially and radially when being pressurized. In HFAM, the outer helical coil restrains the radial expansion of soft microtubule, leading to a lengthwise enlargement of the muscle. HFAM may generate actuation forces by switching between the initial phase and the pressurizing phase. Specifically, it may store elastic potential energy (EPE) by elongating to a certain length, and then by reducing pressure, it converts this EPE to mechanical power. It means that HFAM, in at least some forms, extends to store energy and contracts to generate mechanical force.

In some forms, the HFAM is used without a sheath. In these forms, depending on the nature of specific applications, each HFAM may be arranged in a certain configuration, material combination, and predefined parameters such as outer diameter and original length lₒ. As shown in FIG. 6, mathematically, when a fluid pressure P is applied to HFAM inner channel, it will be lengthened from the initial length lᵢ to length lₚ or a displacement x=(lₚ−lᵢ), which is due to the circumferential constraint by helical coil around the soft microtubule. At this displacement, HFAM accumulates elastic energy. When fluid pressure is removed, HFAM discharges this EPE and returns to its initial phase. If a load is connected to the end of the HFAM, the release of EPE will allow HFAM to apply a contraction force, which is sufficient to bring the load to the desired displacement. The higher pressure that is applied, the higher elastic energy that is stored and thus, a higher contraction force is achieved. Each HFAM has a limit of the elongated length lₘₐₓ to maintain each normal function, corresponding to maximum pressure Pₘₐₓ where it achieves maximum contraction force when this pressure is withdrawn.

In some forms, the disclosure includes using the soft microtubule wherein the outer diameter dₒₜ is larger than the inner diameter d_{ic} of the helical coil. In some forms, this may have an advantage in overcoming the initial nonlinear dead-zone of HFAM, which is due to no change in the motion or force output regardless of an increase in the fluid pressure. As a result, inserting the soft microtubule into the helical coil will shrink its inner diameter dᵢ<dᵢₜ and stretching the length of the silicone tube, lᵢ>lₜ, where dᵢ and lᵢ are the inner diameter of the silicone tube and initial length of the muscle at the initial phase. The outer diameter of the silicone tube after insertion is equal to the inner diameter of the helical coil, dₒ=d_{ic}. Without wishing to be bound by theory, a model of the HFAM with respect to the change of applied pressure P with the coil stiffness k_{c}, Young's modulus of the silicone tube material E, stretching ratio of the HFAM α=lₜ/lᵢ, cross-section area Aₜ of microtubule before insertion. The relation between the output force Fₒᵤₜ or elongation x and applied pressure P is expressed by:

$$P = \frac{α {EA}_{t} ( 1 - \frac{1}{1 + x / {l}_{i}} ) + {k}_{c} x}{\frac{π}{4} {d}_{o}^{2} - \frac{α {A}_{t}}{1 + x / {l}_{i}}}$$

$${F}_{out} = α {EA}_{t} ( 1 - \frac{1}{1 + x / {l}_{i}} ) + {k}_{c} x$$

In some forms, one of the advantages of the disclosed HFAM is that its contraction force output has the component F_{c} from the helical coil, which is higher than existing soft actuators with the same structure. In addition, the use of stretching ratio α also contributes to increasing the contraction force threshold due to an increase in its stored elastic energy.

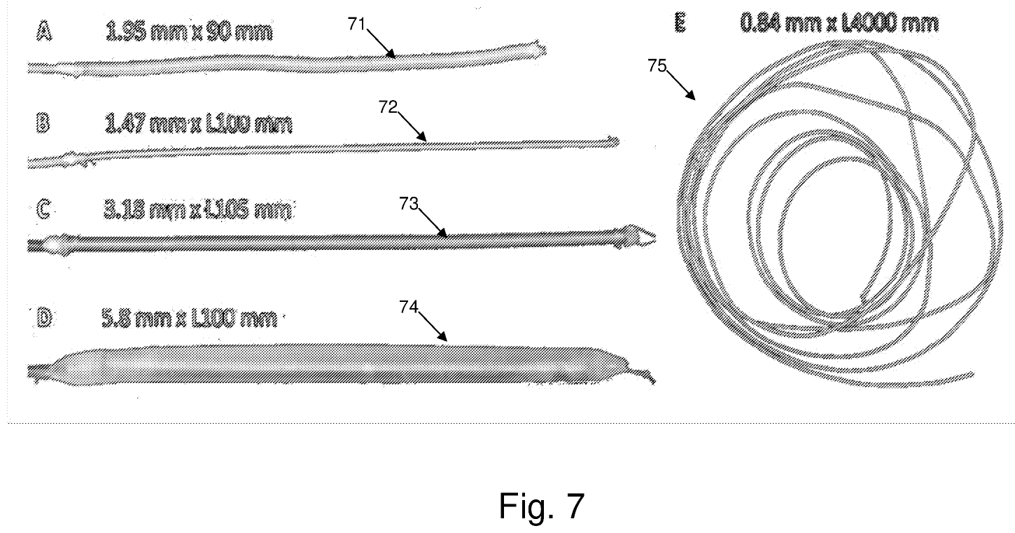

Referring to FIG. 7, disclosed are several HFAM prototypes 71, 75. A. Silicone rubber tube and fishing line (PVDF) coil. B. Silicone rubber tube and stainless steel coil. C. Latex rubber tube and stainless steel coil. D. The muscle in C after coating a layer of Ecoflex. E. A long, miniature muscle made from silicone rubber tube and stainless steel coil.

To characterize the performance of the novel HFAM in various aspects, a measuring system was developed that consists of a DC-motor, amplifier, pressure sensor, miniature syringe, linear ball screw, encoder, force gauge, and real-time controller. This system may generate input pressure up to 2 MPa to the HFAM. In the experiment, one end of HFAM is directly connected to the syringe while the other end is connected to the encoder or force gauge where the real-time controller decodes the recorded signals.

Experimental characterization of a single HFAM was performed in both elongation and force mode. A sinusoidal signal was applied to the syringe plunger, which controls fluid volume or pressure to induce elongation or contraction force of the HFAM and construct the relation between the input (volume and pressure) and the output (elongation and force). To measure the output displacement, we used a rotational encoder which is connected to one end of the HFAM via a linear slider and an elastic string. For force measurement, we firstly elongated HFAM to a certain length and connected its one end to a load cell. Sinusoidal wave signals with a frequency of 0.1 Hz were generated by the piston movement.

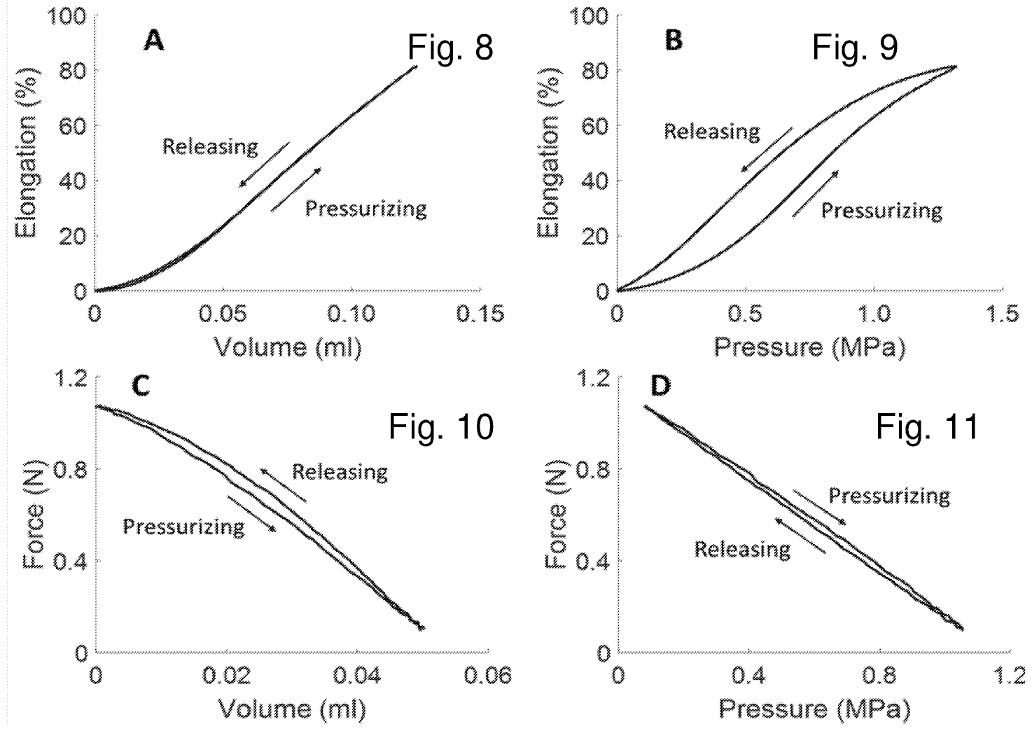

Experimental results for both testing modes are given in FIG. 8 to FIG. 11. With FIG. 8 and FIG. 9 showing an elongation test and FIG. 10 and FIG. 11 showing a force test). It was observed that the 80 mm long HFAM could reach approximately 81.4% of elongation with approximate 0.125 ml of supplied fluid volume (water in this case), which corresponds to around 1.32 MPa in maximum pressure. Experimental results also revealed that the hysteresis profiles in forward and backward directions appear noticeable distinction. In the volume-elongation (FIG. 8), the pressurizing phase profile has almost resembled the releasing phase profile. In contrast, the pressure-elongation (FIG. 9) has a relatively wide hysteresis profile, in which a greater gap exists between the pressurizing and releasing phase. For the contraction force response, the hysteresis profile has a reverse proportional relationship with respect to the change of input volume and pressure. When pressurizing to 86.3% of elongation, HFAM could generate a force of 1.08 N. There are relatively narrow gaps in the hysteresis profiles of volume-force (FIG. 10) and pressure-force (FIG. 11). A nearly linear relationship between force and pressure at both pressurizing and releasing profiles may be observed in FIG. 11. Throughout the two series of experiments, there are settled relationships between each individual output (elongation and force) and the input power (volume and pressure). To better understand the HFAM performance in terms of storage elastic energy or elongation, we performed another experiment to establish the relationship between maximum elongation and output contraction force. We used similar HFAM with 80 mm long. We gradually increased the elongation from 10% to 180% with a step of 10%. The maximum force values were collected at each blocked elongation. There is a nearly linear relationship between the elongation and contraction force. The force has a proportional relation with the elongation, which approximately follows the disclosed model given by the equations. When the initial elongation of the HFAM increases, it accumulates more elastic energy, leading to a stronger force when releasing the pressure.

Durability tests were conducted to demonstrate the reliability and reusability of the HFAM, durability for both the elongation and force durability tests is carried out. We applied sinusoidal signals to the DC micromotor over time period of 2000 seconds. Experimental results revealed that both elongation and force data almost remained stable with respect to the input signals. However, the signal amplitudes are gradually shrinking over times. The force signal exhibits a larger shrinking rate compared to the elongation signal. After 2000 seconds, force shrinks 11.9% from 0.957 N to 0.843 N, while that of elongation is only 1.9% (elongation reduces from 81.36% to 79.82%). This shrinking amplitude of signals may be explained by the inherent elastic properties of the HFAM. Both the helical coil and microtubule are elastic components that are affected by fatigue after performing multiple periodic cycles of stretching. A huge difference in the shrinking rate of elongation and force is mainly because of the nature of the experimental setup. The HFAM in the elongation test was being stretched to the pressurizing phase and then restoring its initial phase, where the HFAM was temporarily relaxed. In contrast, the force test maintained a constant elongation of the HFAM while the data collection process was running. As a result, the accumulated fatigue of the HFAM was augmented faster in the case of force test than elongation test.

The maximum elongation of HFAM was also reviewed. An HFAM with an initial length of 50 mm was used to test the elongation limit. The HFAM was switching in between the initial phase and pressurizing phase with a steady increase of signal amplitude at each cycle. In the last full cycle, HFAM reached an elongation of 224% and successfully return to its initial phase. Subsequently, the HFAM touched its elongation at break of 246.8% before malfunctioning. We performed 10 trials to validate the experiment. Results showed that the failure mostly originates from the interface between the rigid fluid transmission tube and soft microtubule. It means that a stronger adhesive glue should be used.

We also carried experiments to characterize the lifting performance and energy efficiency of HFAM. To demonstrate the scalability, we used two different versions of HFAMs (each has the original length of 50 mm). We performed lifting tests where HFAMs were connected to their relevant weights. We supplied input pressure to HFAMs to store elastic energy while the weights are simultaneously lowered. Thereafter, we withdrew the fluid pressure to induce the contraction force to lift the weight up. The single 50 mm long HFAM weighed 0.28 gram could lift a load of 100 grams, which is about 357 times larger than its weight, with a stroke of 64 mm (95.5% of elongation). Likewise, the single 50 mm long HFAM could lift a load that is 352 times heavier than its mass (500 grams compared to 1.42 grams) but achieved a lower stroke and elongation at 42 mm and 67.7% respectively. We also conduct the energy efficiency tests through a series of lifting experiments using three distinguished variants, including two single HFAMs and a one-dimension weaving sheet. Energy efficiency reflects how effective the HFAM to convert energy consumption to mechanical power. It is defined by the ratio between the output and input mechanical power. In the disclosed experiments, HFAM was powered by fluid via a standard 1 ml syringe. Therefore, the input mechanical power is a product of the applied force Fₚₗᵤ to the plunger and displacement of the plunger xₚₗᵤ inside the syringe barrel per moving time tin. The applied force was measured by a force gauge (MARK-10), which directly connected to the syringe plunger. The output mechanical power is a product of a lifting load F_{load} and moving distance (stroke), x_{load}, per lifting time tₒᵤₜ. However, the instant response of the HFAM due to nonlinear hysteresis makes an insignificant deviation between tᵢₙ and tₒᵤₜ. In this test, we try to understand the performance of HFAM with respect to different sizes and strokes. Therefore, we assume that this deviation is small and maybe ignored or tᵢₙ≈tₒᵤₜ. Experimental data of the three variants of the HFAM and their energy efficiency, EE=Pₒᵤₜ/Pᵢₙ=F_{load}x_{load}/Fₚₗᵤ. xₚₗᵤ, are calculated. Experimental results revealed that the single 50 mm long HFAM1 could reach an energy efficiency of 46% while this value is higher than that of the single 50 mm long HFAM2 at 62.7%. Despite more energy consumption, the key point of the HFAM2 to surpass the HFAM1 in terms of energy efficiency is the ability to carry a much higher load. More interesting, a one-dimension weaving sheet made from a single HFAM1 and acrylic yarns outperformed the other two variants by producing an energy efficiency of 69%. The comparison between the disclosed developed HFAMs, and other soft actuator means that the weaving configuration gives better energy efficiency.

By varying the arrangement of HFAMs, a wide range of motions can result, including contraction, elongation, bending, shape-shifting structure and different structures, devices, and robots such as smart garment by knitting and weaving, high-performance muscle by twisting and braiding, planar muscles with omnidirectional motion including radial and axial expansion, reconfigured surface, multimodal locomotion and shape-shifting robots from 2D to 3D, and metamaterial structure.

Although the soft hydraulic filament artificial muscles are scalable and versatile in terms of sizes, specifications, and material combinations, a single HFAM is far from a one-for-all actuation solution. Of course, multiple single HFAM can be combined to produce a higher contraction force if needed. This method is simple yet effective to satisfy numerous actuation force demands. On the other hand, inspired by the human muscles and rope industry where multiple filaments are twisting and braiding to enhance its endurance limit while retaining its flexibility. The exertion force of a single HFAM can be augmented by folding it with or without twisting or braiding it in the lengthwise direction. A twisting soft hydraulic filament artificial muscle (T-HFAM) can be achieved by twisting multiple single HFAM or folding a single HFAM then twisting their segments together or a combination of these two methods. There is a wide range of T-HFAM configurations that depend on the number of segments after folding, the number of turns after twisting, and how many single HFAM are used. Braiding technique in wire and rope technology is required to obtain a braiding soft hydraulic filament artificial muscle (B-HFAM). Several segments of a single SHAM, multiple single HFAM, or T-HFAM are braided together to form an integrated muscle. Braiding techniques may vary from the classic 3-strand braid, the classic multiple-strand braid, and the rope braid. It is worth mention that all folding points must avoid a sharp bend otherwise the fluid will be impeded running throughout the muscle, leading to a significant decline of the muscle performance.

The fundamental working principle of T-HFAM and B-HFAM is identical to the single HFAM. The muscle is firstly at its relaxed condition/initial phase with no reserved energy. When pressurizing by air or fluid, the muscle will elongate and store elastic energy before converting this energy to mechanical power under the releasing phase. It is predicted that the muscle with more segments will produce higher contraction force than those with lesser segments when the same specifications and initial elongation are applied. The explanation for this occurrence is that the more-segment muscle is required more input energy to reach a certain initial elongation. As a result, it will exert more force when releasing. Related to elongation of the muscle, the rule of thumb is that a predefined input volume of fluid will cause the muscle to extend to a certain length. Therefore, the elongation will entirely depend on the original length and the number of segments of the muscle.

Comparison experiments between several variants of the T-HFAM, B-HFAM, and single HFAM were conducted to gain insight knowledge about the output elongation and force of these muscles corresponding with the input volume and pressure.

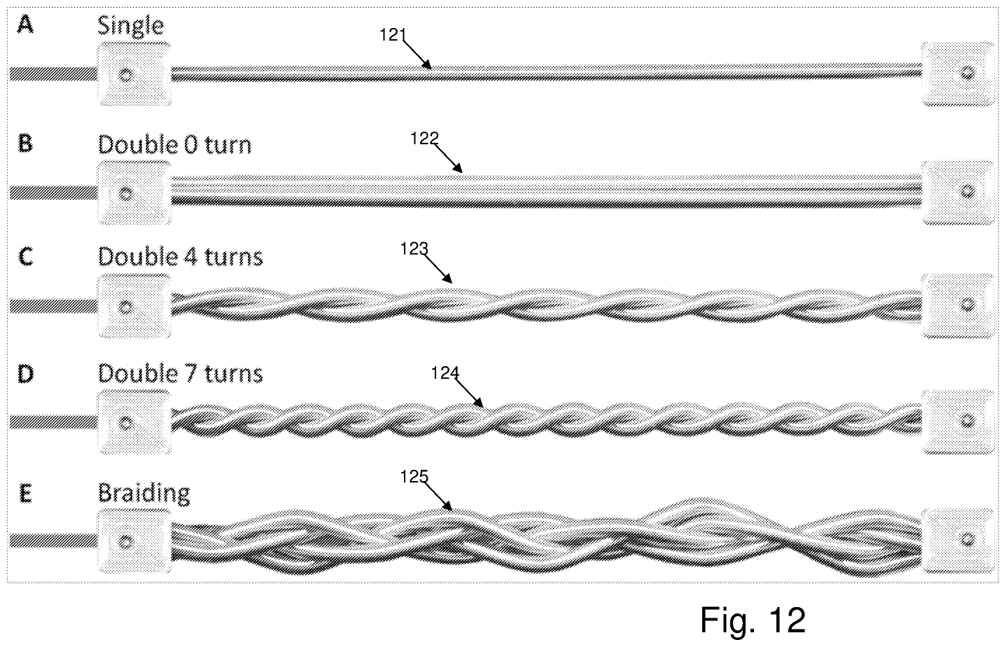

Turning now to FIG. 12, five variants 121-125 were tested. All variants are made from HFAM. A. Single muscle, length 80 mm (121). B (122). C (123). D (124). Single muscle, length 160 mm, double twisted with 0, 4, and 7 turns respectively. E (125). Two muscles, length 160 mm each, braiding in 4 segments. All these variants were HFAM with different lengths and configurations as shown in FIG. 12. A single muscle with one segment 121 belongs to group 1; group 2 gathers two-segment muscles including three variants of the double twisted muscle with different twisted revolution at 0, 4, and 7 turns (122, 123, 124); and group 3 has a four-segment muscle that was braided from two double folding muscle (125). This categorization has the two-fold benefit. Firstly, the peculiar behaviors of the three groups will reveal the influence of the number of segments on muscle performance. Secondly, three variants of group 2 will expose how the number of twisted turns affects muscle function.

The proposed single soft microtubule muscle (HFAM) and its variants of twisting (T-HFAM) and braiding (B-HFAM) may serve as a fundamental soft actuator. The HFAM possesses various advanced properties such as high elongation, high energy efficiency, scalability, versatility, and multifunctionality that enable its involvement in a diversity of robotic applications. The HFAM elongates to store energy and retracts to generate force. The comparable characteristic between the HFAM and the human muscles empower the HFAM to potentially become an ideal candidate for artificial muscle applications.

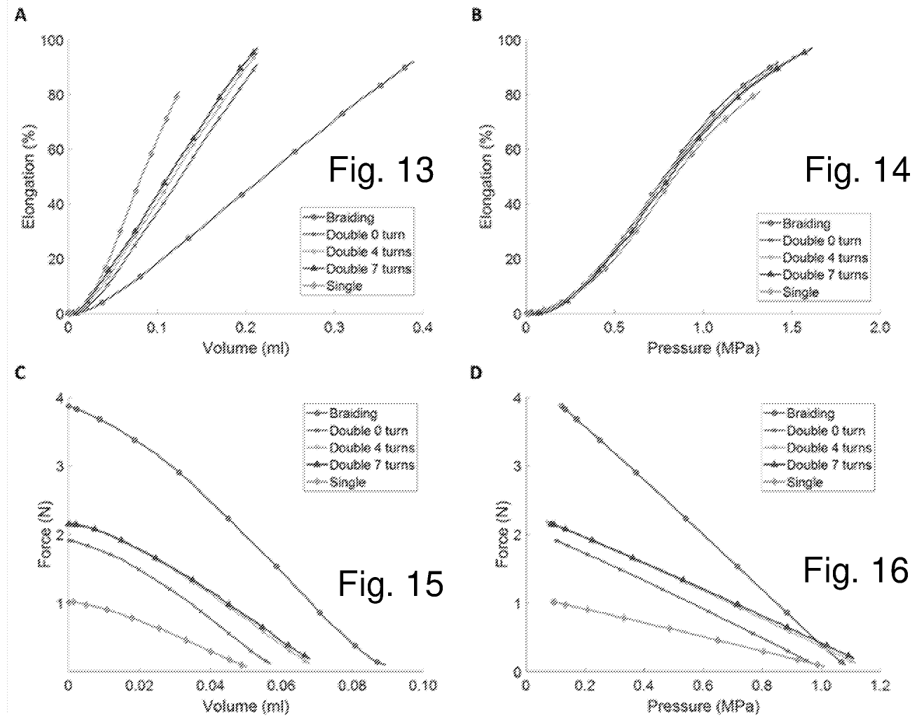

FIG. 13 to FIG. 16 shows the experimental results, where each hysteresis profile is presented in a half cycle to ease the comparison illustration. The elongation lines are at the pressurizing phase, whereas the force lines are at the releasing phase. At a glance, relationship profiles of all five variants have the same tendency. Elongation is proportional, while force is reverse proportional to input volume and pressure. Specifically, with the same amount of input volume e.g., 0.12 ml, the single HFAM achieved the highest elongation at about 78%, following by three variants of the T-HFAM at the vicinity of 50%, and the B-HFAM stood at the lowest with nearly 24% (FIG. 13). This happened because all variants have a similar initial length but different original lengths. It is estimated that they were accumulated an equally elongated length when being supplied with the same amount of fluid. As a result, those are longer will have lesser elongation and vice versa. Three variants of the T-HFAM possess a slight divergence that those with more turns have a little higher elongation. Twisted revolutions decrease the inner diameter of the muscle, therefore the muscle may be stretched a bit further with the same amount of volume. However, experimental data revealed that the influence of the number of turns on elongation is minor. Interestingly, regardless of the obvious distinctions between the five variants in terms of length and configuration, their pressure—elongation profiles remain proximity (FIG. 14). This is strong evidence to confirm the pressure—elongation relationship of the HFAM mostly dependent on material properties and parameters of the muscle components rather than the length and configuration. Results in FIGS. 15 and 16 justified the prior prediction about the influence of the number of segments on the muscle contraction force. When pressurizing to 87% of elongation, the B-HFAM exerted the highest contraction force at roughly 3.9 N, three variants of T-HFAM fluctuated from 1.9 N to 2.2 N, and the single HFAM held the lowest at about 1 N. It is clear that the number of segments of the muscle greatly affect its contraction force. Furthermore, two T-HFAM variants with 4 and 7 turns had the same maximum force at 2.2 N while the remaining T-HFAM (double 0 turn) reached a little lower at 1.9 N. The interaction between two segments of the muscle when twisting reduces force loss and enhances the energy efficiency. However, this circumstance only promotes a modest to negligible force increase. The convergence of all lines in FIG. 16 reinforces the conclusion drawn from FIG. 14 that disregarding the length and configuration, all the five variants are required about 1 to 1.1 MPa to attain the initial elongation at around 87%.

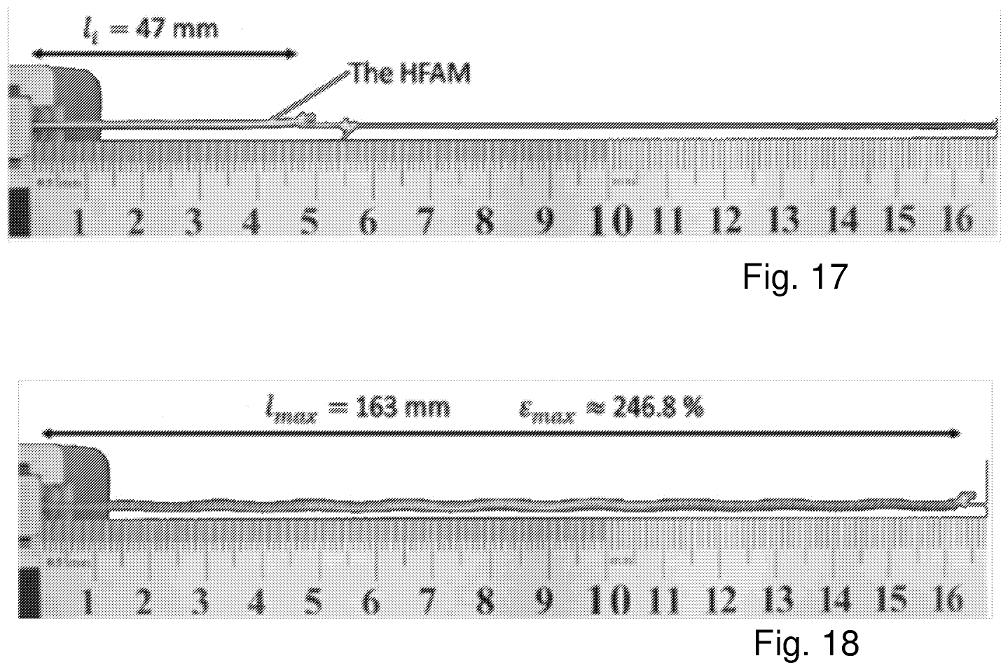

FIG. 17 and FIG. 18 shows rest and maximum elongation of a single HFAM. A 50 mm long HFAM1 was used.

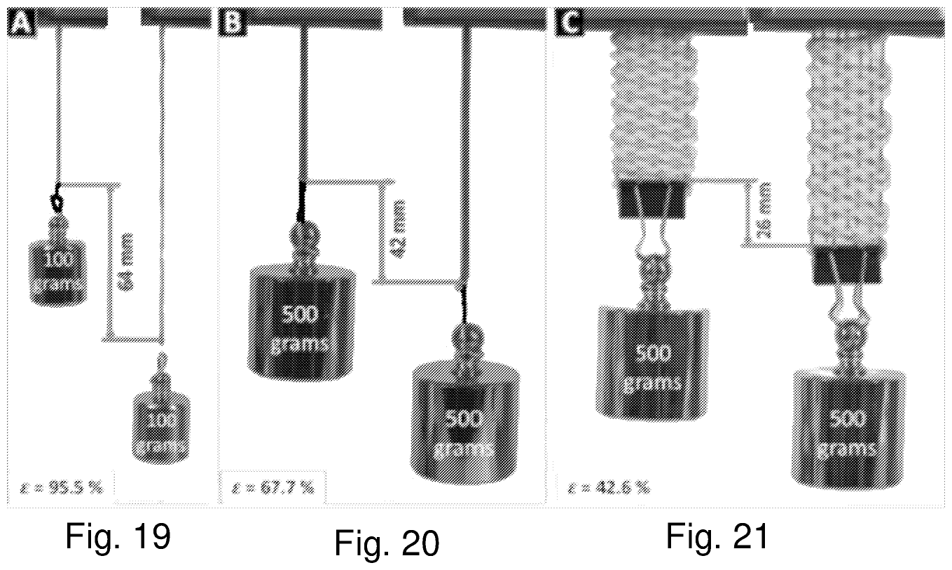

As shown in FIG. 19 to FIG. 21, the weaving HFAM (W-HFAM) was tested for lifting capability and energy efficiency. Two ends in the lengthen direction of a one-dimension weaving sheet were fixed to a fixture and a load. By switching between the pressurizing phase and releasing phase, the weaving sheet carried the load downward and upward, respectively. A one-dimension weaving sheet weighs 2.6 grams could lift a 500-gram load, which is 192 times heavier than its mass, with a stroke of 26 mm and achieved a remarkable energy efficiency at 69%.

The W-HFAM is a soft, flexible, and active fabric that may produce elongation, area expansion, and actively provide contraction force. These advanced characteristics of the W-HFAM will benefit a wide range of robotic fabric applications. The following part introduces two typical applicable functions of the W-HFAM, including compression garment and shape transformation.

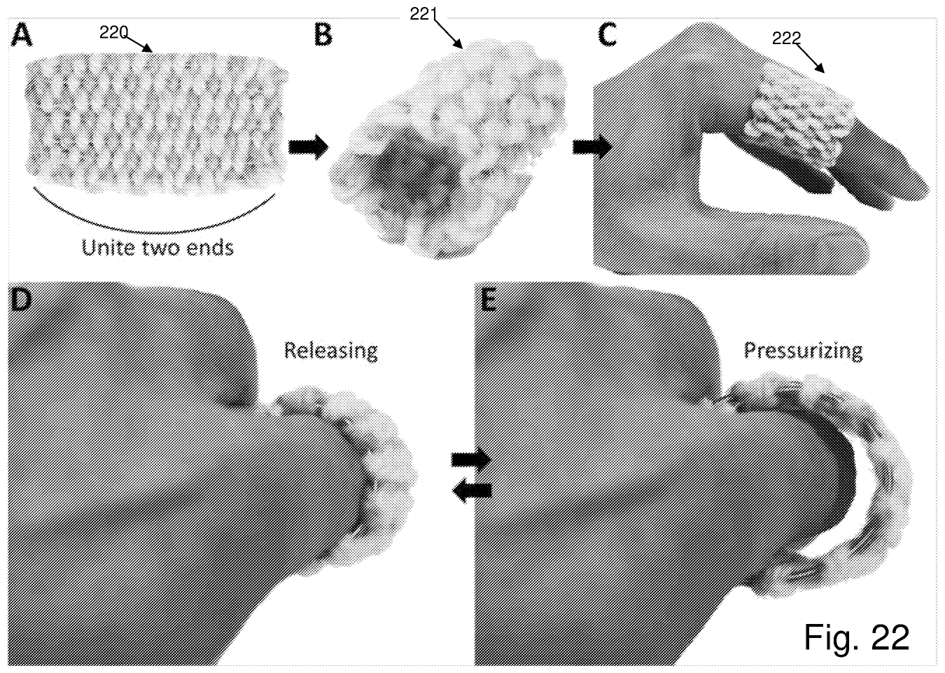

FIG. 22 illustrates photographs of a prototype of a compression sleeve for a finger. A cylindrical compression sleeve was produced by merging two edges in the stretching direction of a one-dimension weaving sheet 220. That is the axial weaving technique of a single HFAM in the helical configuration and acrylic yarns. This hollow device 221 was then inserted to cover an index finger 222. The diameter of the compression sleeve may be continuously controlled by increasing or decreasing the input pressure. The diameter of the compression sleeve may be continuously controlled. As a result, the compression force generated by the sleeve to the finger may be fully manipulated in terms of amplitude and frequency that follows predefined therapies. Thanks to the scalability of the HFAM, the compression sleeve may be customized fabricated to suit the human arm, leg, body, or other parts. These sleeves will serve as massage therapies to relieve pain, improve blood circulation, and relaxation. In addition to the sleeve, the weaving sheet may be further developed to become a wearable glove or suit for human uses.

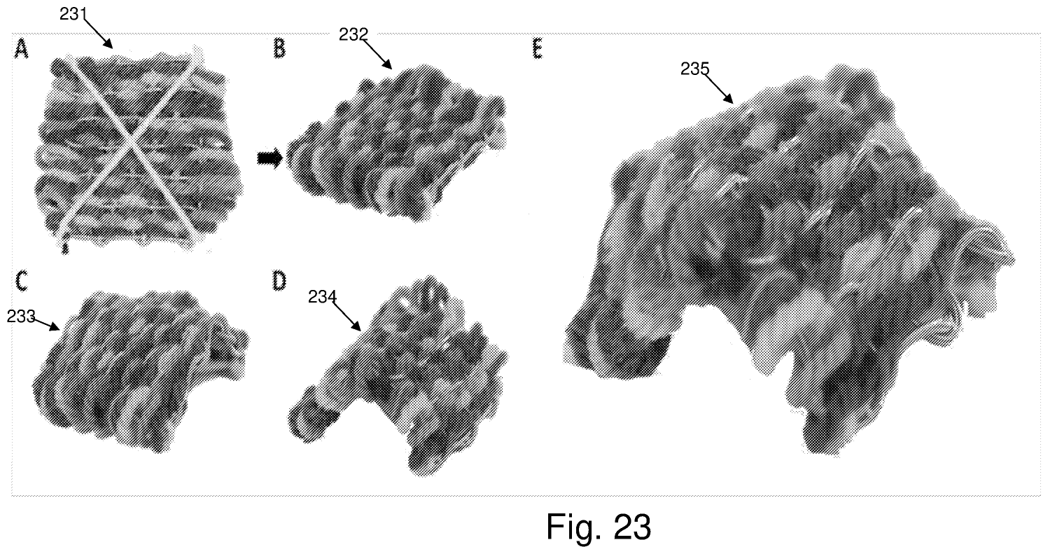

FIG. 23 presents a simple concept of shape transformation based on a two-dimension weaving sheet. The initial phase of the weaving sheet is a square shape in a 2D plane 231. The figure shows the shape transformation of a two-dimension weaving sheet. A. Diagonal constraints of the sheet 231. B. Flip the sheet to the ready position (initial phase) 232. C. D. Pressurizing in each dimension 233, 234. E. Pressurizing both dimensions, the 2D sheet is fully transformed into a 3D structure 235. The sheet is made from two HFAMs (350 mm long each) and acrylic yarns with the original dimension of 46×47 mm.

By constraining the diagonal extension at one surface and applying pressure, the weaving sheet will form a dome that looks like a 4-legged robot 235. The transformed shape from 2D to 3D of the weaving sheet may be controlled by numerous strategies. Firstly, deciding which muscles to actuate: one active one passive and vice versa; same phase for the two; or overlapping active between them. Secondly, the intensity of the input pressure. Each in these endless variations will create a unique 3D structure of the two-dimension weaving sheet. The shape transformation ability of the W-HFAM paves the way for the development of robotic locomotion or other active fabric applications.

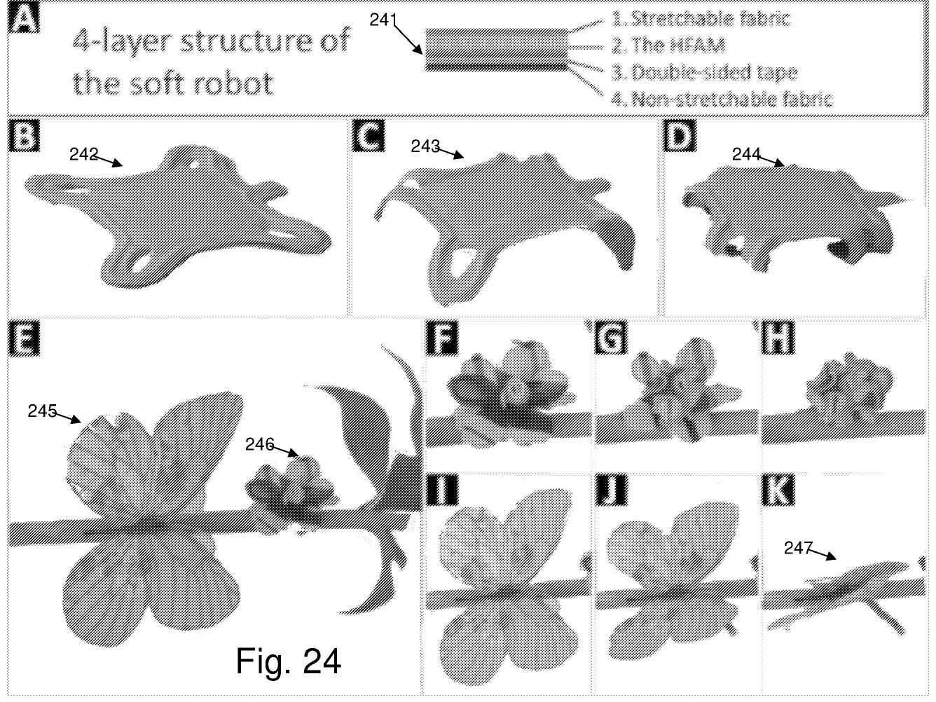

Referring to FIG. 24, disclosed are bio-inspired soft robots. A. The principle structure of embedding and routing HFAM in fabric layers by double-sided tape 241. B-D. A 4-legged robot at the initial phase 242, medium pressurization 243, and high pressurization 244, respectively. E. Butterfly robot and flower robot 245 with embedded HFAMs under the wings and petals. F-H (246). The flower robot at the initial phase, pressurizing only the top-layer petals, and pressurizing both layers of petals, respectively. I-K. The butterfly robot at the initial phase, medium pressurization, and high pressurization, respectively.

The HFAM was integrated into fabric layers by double-sided tape. This method fully constrains the non-stretchable layer while retaining the function of the stretchable one, leading to a purely bending motion of the obtained structure without the helical twisting effect. The long and flexible HFAM allows it to freely route on 2D and 3D surfaces to create endless desired shapes or embed to sheet-like passive objects such as fabrics, papers, and polymer sheets. This fast, cheap, and versatile fabrication methodology enables multifunctionality, on-demand, and mass production of soft robots.

FIG. 24 illustrates the principle 4-layer structure 241 to create bio-inspired soft robots and reconfigured objects from passive to active. The double-sided tape is used to fully anchor the HFAM to the non-stretchable fabric layer. The stretchable fabric layer stabilizes the routed HFAM and also serves as a cover to protect the HFAM. In some cases that require a slight bending force, the stretchable layer is optional. FIG. 24 B-D (242, 243, 244) present a 4-legged robot made from two layers of fabric and is power by a single HFAM that is routed around the outer boundaries. The robot was mechanically programmed to bend only at its legs by using double-sided tape to reinforce other parts of the robot (not at the legs positions). When the HFAM is supplied by fluid pressure, only the leg segments can bend, resulting in the lifting of the robot body. The bending angle of the legs can be controlled by the intensity of the input pressure. The 4-legged robot can exert locomotion if its feet are equipped with directional patterns or using multiple HFAMs with locomotion operation strategies. The locomotion soft robots are needed in various tasks including rescue missions from bushfires, collapse building, or hazardous environments, and drug delivery robots for medical applications.

FIG. 24 also introduces other bio-inspired soft robots 245, 246 by embedding HFAM into passive objects to make them active and controllable. The fabric flower has two layers of petal and each layer has five petals. A single HFAM was sticking beneath each petal layer following the outer boundaries of all petals. At the initial phase, the flower is fully blooming, in which all petals are entirely open. Upon pressurization, the HFAM will cause the bending motion of petals, making them close. Two HFAMs control the motion of two petal layers independently while five petals in the same layer are curving at the same time. Like the flower, the butterfly was produced by a thin fabric sheet and a routing HFAM attached under its wings. The butterfly's wings will bend upward when pressurizing the HFAM and return to the balanced position by releasing the pressure. Both left and right wings of the butterfly are flapping at the same time because they are controlled by only a single HFAM. The ability to transform passive sheets to active ones of HFAM opens a whole new possibility to manipulate objects. By only routing and sticking a single HFAM in any shape on sheet-like objects to make it active and controllable. This fast and simple method is useful in producing compact and deployable devices for space missions, construction, and industry. It can be used to create bio-inspired robots that provide biomimetic motions for decorations and fashion design.

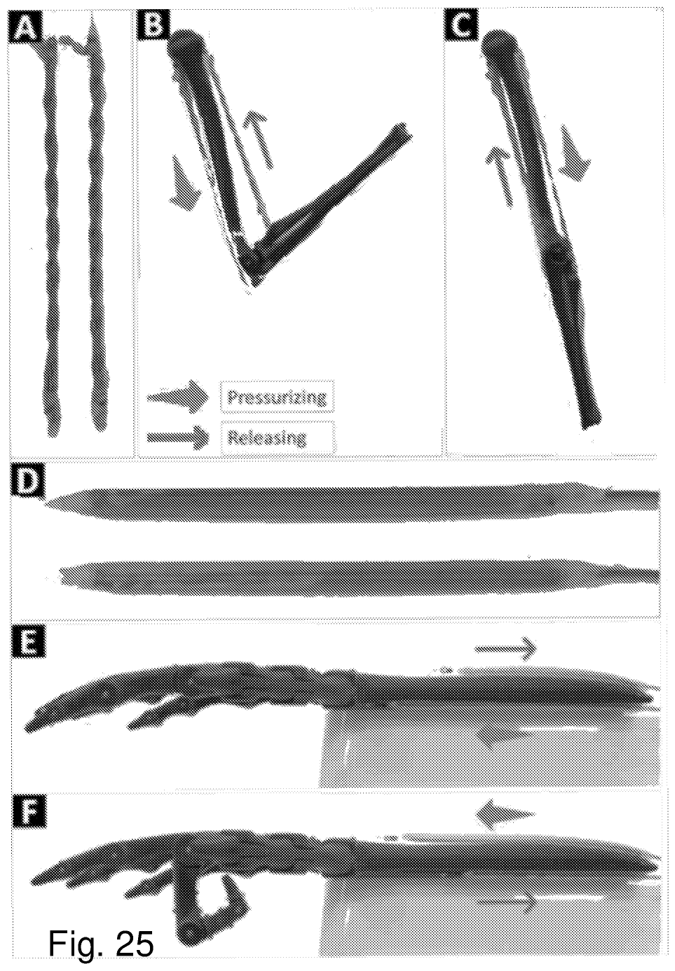

FIG. 25 demonstrates the usage of the HFAM as artificial muscles to mimic movements of the human elbow and index finger. To exhibit the versatility of the HFAM, two different types of artificial muscles were produced. The first one was made from a 600 mm long HFAM with the quadruple twisted configuration to power the elbow while a single 90 mm long HFAM coated with a thin layer of EcoFlex™ was fabricated for the second one to drive the finger. Each type had a pair of muscles to facilitate bi-directional rotation of joints. In a pair, when one muscle is being pressurized, the other must be releasing and vice versa. The muscles are switching between the pressurizing phase and releasing phase in sequence to manipulate the joints. Because the artificial muscles exert contraction force, the controlled links will be driven toward the muscle that is at the releasing phase. In the elbow application, two artificial muscles mimic the human bicep and tricep to maneuver the forearm in its full range of rotational motion. The index finger model is under-actuated, consists of three controlled links and three rotational joints. Like the human hand, the artificial muscles do not connect directly to the links but via two tendons that run alongside these links and joints. Despite under-actuated, the two artificial muscles could complete the full range of flexion and extension of the index finger. The model of elbow and finger simplified the actual human anatomy to some extent, such as the model replaced the complex elbow and finger joints with the simple rotational joints, and the model suppressed all other muscles, tendons, and many features that may affect the links' movement. The artificial muscles may be scaled up to power exoskeleton suits and exo-glove, or integrated into garments for spinal assistance.

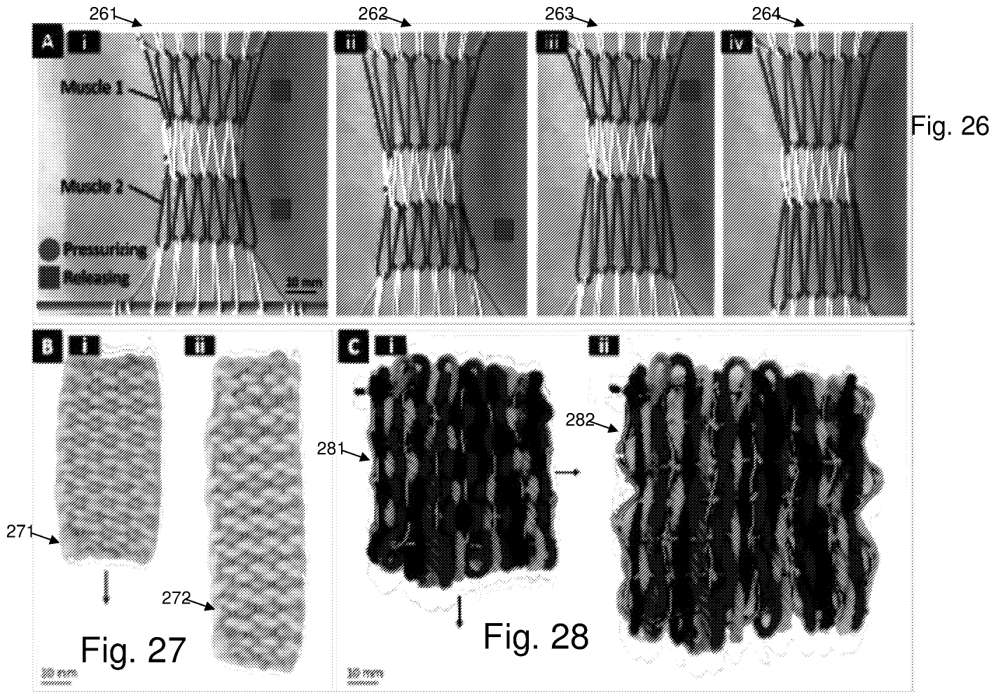

FIG. 26 to FIG. 28, shows knitting and weaving HFAM. A. Knitting sheet made from two single HFAMs and elastic strings at the initial phase 261 (i); pressurizing the muscle 1 262 (ii); pressurizing the muscle 2 263 (iii); pressurizing both muscles 264 (iv).

FIG. 27 illustrates a One-dimension weaving sheet made from a single HFAM and acrylic yarns at the initial phase (i) 271 and pressurizing phase (ii) 272. C. FIG. 28 illustrates a two-dimension weaving sheet made from two single HFAMs and acrylic yarns at the initial phase (i) 281 and pressurizing phase of both muscles (ii) 282.

Multiple smart garment prototypes may be made from embodiment HFAM1, acrylic yarns, and elastic strings by the traditional knitting and weaving techniques. A knitting soft hydraulic filament artificial muscle (K-HFAM) is a planar chain-like structure that was produced from a combination of HFAMs and elastic strings by knitting method. The knitting sheet was created row-by-row by inserting and looping the current filament into the previous loops. The knitting sheet can be made entirely from one type of filament (HFAM) or interleave with other fibers. Here, we combined HFAM with elastic strings to demonstrate the versatility and customization capability of the disclosed muscle. The produced knitting sheet will elongate and exert the contraction force in the axis that perpendiculars with the knitting direction. Due to the loose loop construction of the knitting technique, the knitting sheet is highly extensible, suiting those applications that require one-dimension lengthen. A weaving soft hydraulic filament artificial muscle (W-HFAM) is a crossing pattern of filaments and can be achieved by interlacing a single or multiple HFAM with various types of wires such as yarn, fiber, and thread. The properties of the W-HFAM are influenced by the muscle, the participated wires, and how the weaving sheet is manufactured.

FIG. 26 to FIG. 28 introduces the prototype of a one-dimension weaving sheet made from a single HFAM and acrylic yarns. The single HFAM was folded several times but instead of gathering, it expanded in a 2D plane. The zigzag shape of the folding HFAM was then secured by the acrylic yarns by weaving technique. The one-dimension weaving sheet will expand in only one direction upon pressurizing. A two-dimension W-HFAM was produced from two single HFAM and acrylic yarns, whose prototype is illustrated in FIG. 28. Segments of the two zigzag HFAM were interlacing with each other to form a weaving sheet. The acrylic yarns filled all porous to stabilize the 2D shape. In some cases, rubber bands are required at intersections to sustain the integrated sheet. The two-dimension weaving sheet can produce elongation in two perpendicular directions, resulting in area expansion.

The knitting sheet prototype provided around 31.2% of elongation when pressurizing both participating HFAM while the one-dimension and two-dimension weaving sheet could generate 64.9% of elongation and 152.1% of area expansion, respectively. Like a single HFAM, the knitting and weaving sheets will restore their initial shape when releasing the pressure.

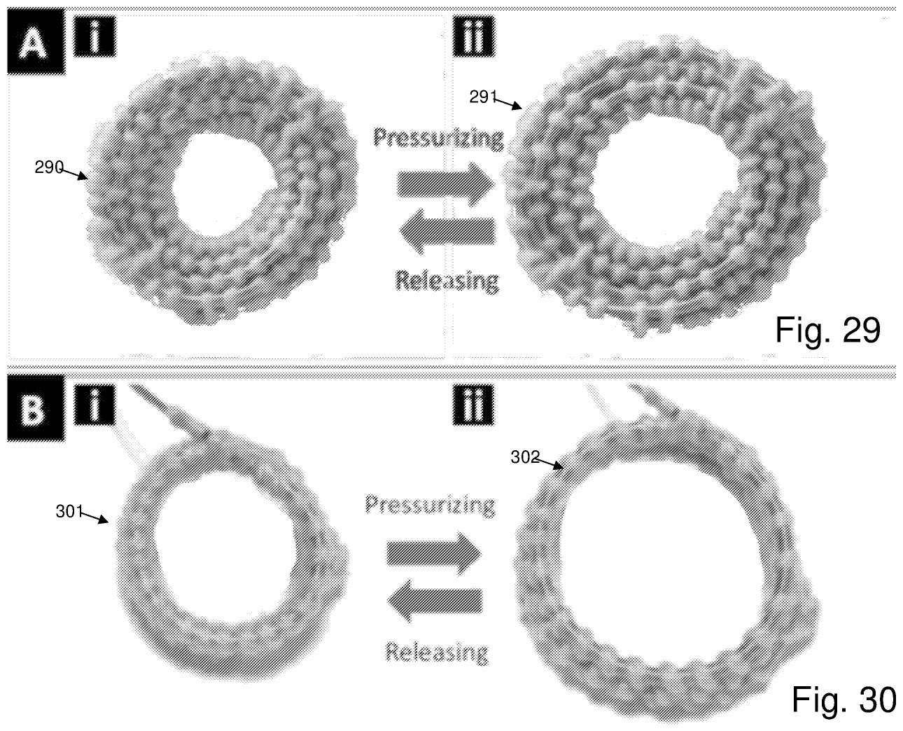

Turning now to FIG. 29 and FIG. 30, the elongation and contraction motion of a long and soft HFAM can be configured to provide radial expansion motion by spiral arrangement. There are two types of radial expansion configurations that are presented. The first one 290 is a 2D spiral arrangement of HFAM to form a planar washer-like shape (FIG. 29). The spiral HFAM is secured by radial weaving of acrylic yarns. When pressurizing 291, the longitudinal lengthen of HFAM results in the radial expansion of the device, in which both inner and outer diameters are increasing. The washer shape will return to the initial dimension when the pressure is released. FIG. 30 illustrates a second configuration 301 as a 3D hollow cylindrical device made from the helical arrangement of HFAM and axial weaving of acrylic yarns. The spring-like configuration of HFAM enables it to transform the elongation motion to radial expansion, leading to an enlargement of the cylindrical diameter upon pressurizing 302. The device will restore its initial shape by releasing the input pressure. Acrylic yarns in both configurations play the role of a flexible linkage element to stabilize and maintain the devices' appearance. Similar to basic HFAM, radial expansion devices expand and store elastic energy in the pressurizing phase and exert force in the releasing phase. However, this contraction force is the centripetal force that happens at every circumferent points of the devices.

The radial expansion motion and centripetal force of these HFAM structures are desired in emerging robotic fields. First, the HFAM-based assistive compressive medical devices can effectively restore the function of human organs with diseases such as heart or bladder failure. The flexibility and versatility of HFAM permit a better conformability to the complex tissue surface, enabling high effectiveness to support the organ as it can mimic the complex motion of these organs in a biomimetic fashion. It will overcome major limitations from existing devices and technologies to support a large number of patients. Second, it is perfectively suitable to create robotic fabrics or smart garments such as adjustable garments and compression sleeves for arms, legs, or body to serve as massage therapy to relieve pain or improve blood circulation. These devices have a large market ranging from hospitals, general practitioners, and individuals as sportsmen, workers, or the elderly. Third, this mechanism will inspire the development of advanced medical tape, implantable biomedical valves, and camera lens.

Heart Assist Device

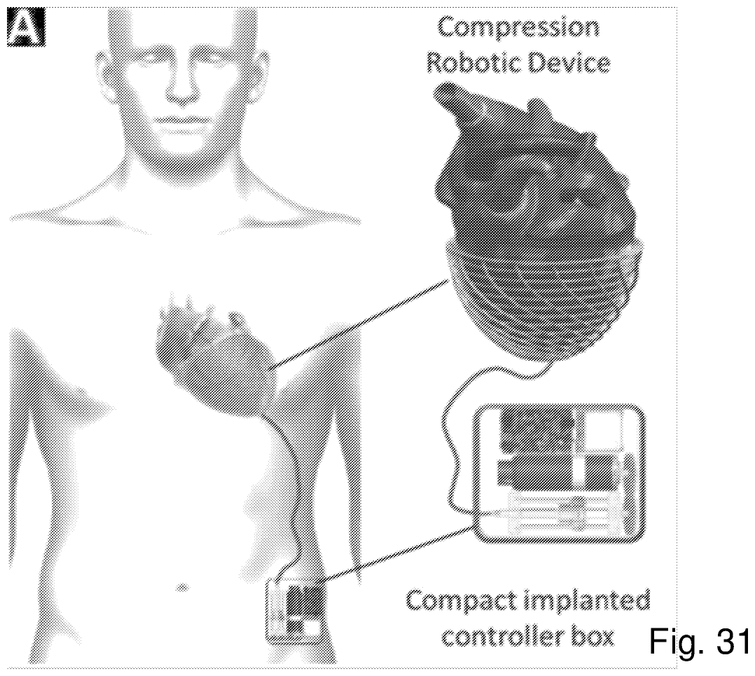

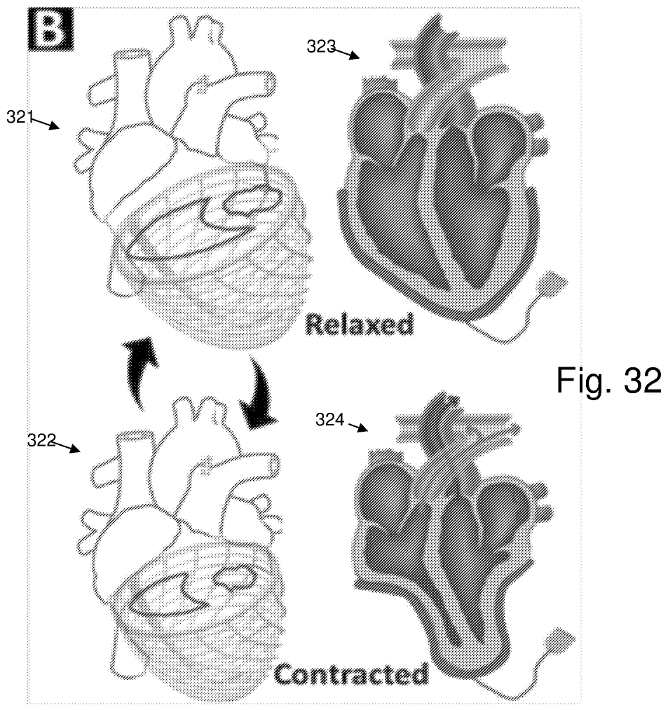

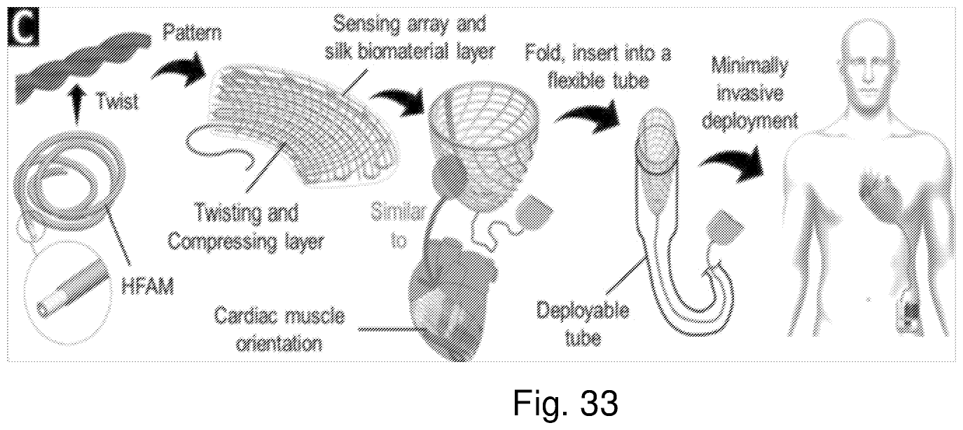

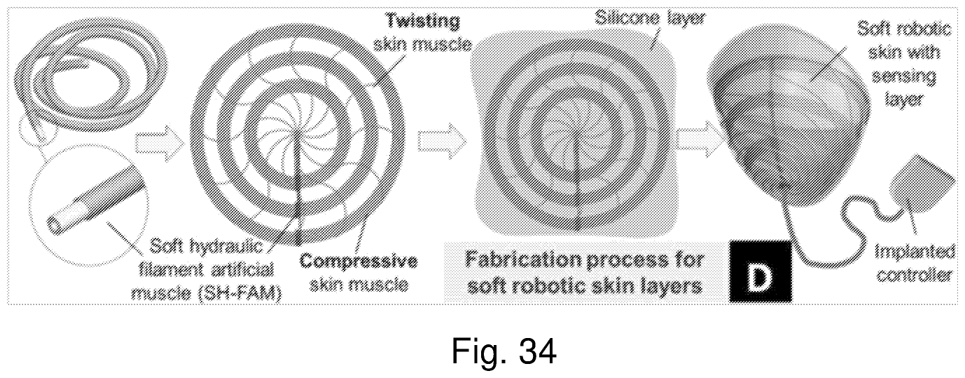

FIG. 31 shows design of a compression robotic heart assist device (CRD), illustrating the position overview of the CRD to the human body. FIG. 32 illustrates the two working phases. FIG. 33 illustrates schematically the fabrication and implantation process of a first embodiment. FIG. 34 illustrates the fabrication and implantation process of a second embodiment 2.

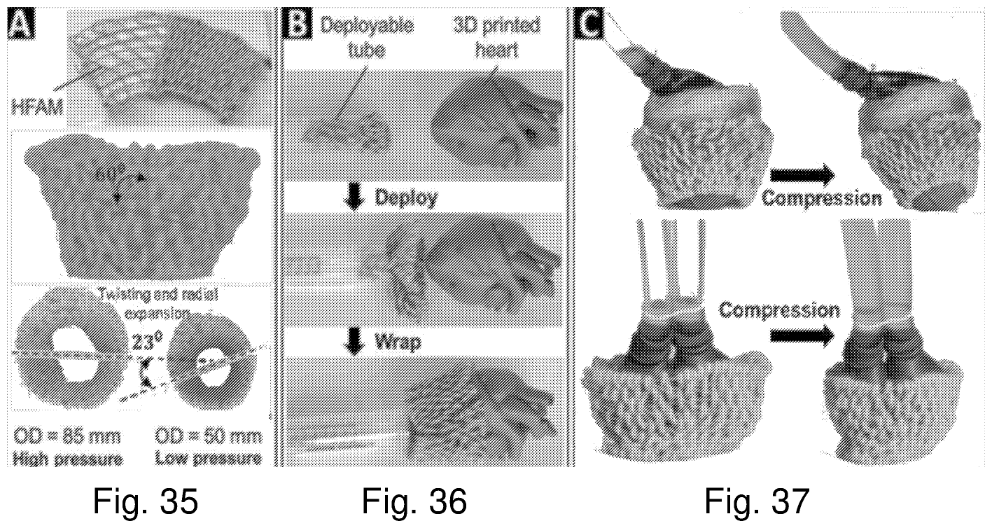

FIG. 35 to FIG. 37 shows the formation of compression robotic device (CRD) prototype.

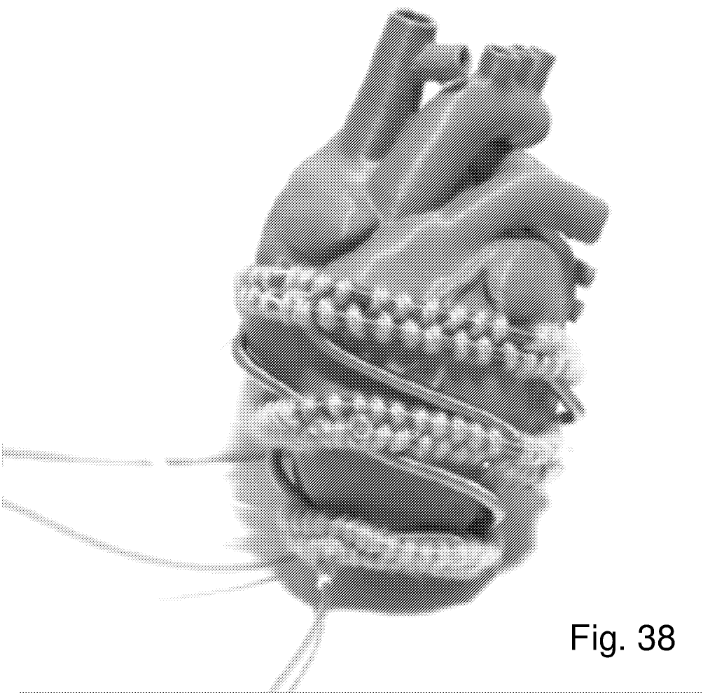

FIG. 35 illustrates the CRD made from a single HFAM and acrylic yarns by the weaving technique; it can provide twisting and radial expansion simultaneously. FIG. 36 illustrates the deployment process of the CRD to cover a 3D printed heart. FIG. 37 illustrates the compression tests with a fresh porcine heart (top) and rubber balloons (bottom). FIG. 38 illustrates wrapping of embodiment 2 for robotic compression device on 3D printed heart.

Heart failure is the inability of the heart to pump sufficient blood to the body, leading to significant morbidity and mortality. By 2030, heart failure will afflict approximately 90M people globally that include more than 0.75M Australians at annual national healthcare cost of $3.8B. In clinical settings, patients with end-stage heart failure are frequently referred for heart transplantation. However, the availability of donor's hearts is limited, and this results in the death of many patients awaiting transplantation. To assist the failing heart, ventricular assist devices (VADs), which withdraw blood from the heart and then pump it into the aorta and pulmonary artery are often used for life-prolonging therapy because they normally remain implanted for the rest of patient's life. Despite advances, the risk of bacterial infection, thromboembolic events, and bleeding resulting from direct contact between the blood and nonbiologic device components are still common in VADs. Alternative therapeutic options, including stem cell therapies or passive restraining devices, have been used, but remaining challenges include engraftment, mobilization, poor cell survival or inability to adjust the restraint level to obtain the desired cardiac output.

Recently, direct cardiac compression devices (DCCDs) have been proven to restore proper heart motion, which undergoes substantial deformation with each contraction. Advances in robotic technologies have enabled the development of several DCCDs such as PediBooster (ABIOMED Inc., USA), HeartPatch (Heart Assist Technologies St, Leonards NSW, Australia), Epic-Heart (Wellcome Trust, Corinnova), or soft robotic sleeve (Harvard, MA, USA) which are placed around the heart to augment the ejection fraction in systole without contacting the blood, offering fewer complications and therefore a safer option than conventional VADs.

In one embodiment, the HFAM-based compression robotic device (CRD) is a soft, anatomically conformal, collapsible, and minimally invasive DCCD that can address these shortcomings. The device is able to induce both helical and concentric motions using a single HFAM with embedded biocompatible materials, soft sensing network, and miniature controller that can surgically be implanted under the patients' skin (FIG. 31). In addition, it will adjust its size to adapt to different patient's hearts and automatically synchronize with the heartbeat to augment the cardiac function. The proposed CRD (FIG. 31) is a thin-film soft robot that surrounds both ventricles of the heart. Unlike existing approaches, it is designed in the form of a collapsible and self-deploying structure, enabling minimally invasive delivery via a flexible deployment catheter inserted through the left rib cage. Also, the CRD is created by a single HFAM, which is arranged in a special pattern to both compress and twist the heart, mimicking the natural heart muscles. The mesh-like structure of CRD and novel silk biomaterial are expected to increase the heart-device contact, which is monitored by a real-time force sensor array. As HFAM only requires a small fluid volume to operate, it can be driven by a compact hydraulic controller via microtube and micro-motor which is significantly smaller than existing state-of-the-art systems. In some forms, the proposed CRD will be programmed to synchronize with the heartbeat via an intelligent closed-loop controller that does not impede the heart function when the device is inactive. It also affirms safe synergistic interaction with the heart and can be customized to adapt to patient-specific needs while maintaining desired cardiac output and avoid using bulky wires and tubes crossing the skin barriers. The system can serve as a bridge to transplant for heart failure patients, leading to a substantial shift in this field with positive impacts on practice and patient outcomes.

The CRD consists of a high density of soft sensor networks to monitor the heart-device interaction, active thin-film actuation layers where a single twisted HFAM is arranged in helical and circumferential patterns, and an implanted controller of DC micro-motor, miniature syringe, and microelectronics. The CRD will be folded and inserted into a deployable tube, which subsequently will be delivered to the failing heart via the left rib cage, allowing minimally invasive implantation.

FIG. 35 presents a prototype of the CRD made from a single HFAM and acrylic yarns by a special arrangement (fan-shape) of the weaving technique. The obtained sheet was folded and sewed to form a 3D funnel shape that could produce radial expansion up to 70% and twisting motion up to 23° simultaneously when pressurizing. Subsequently, it generated around 8 N contraction force when releasing the input pressure. As shown in FIG. 36, the CRD was successfully folded, inserted, and deployed to wrap around a 3D printed heart via a deployable tube. It also ejected a volume fraction of 41.2% on dual silicone balloons and 28.9% on a fresh porcine heart filled with an inserted balloon (FIG. 37). To obtain thin-film, collapsible CRD with a tuneable compression force for minimally invasive delivery, we reduce the size of HFAM while increasing its generated force using stronger constrained coil, which is made from stainless steel microwires and a high strength microtubule which is fabricated by a rolling coating process of Nusil elastomer. The HFAM is then integrated into a thin membrane (Ecoflex) to form an active robotic band (total thickness <1 mm). Later, we wrap this band around a 3D printed heart (size is similar to failing heart) in the desired orientation, mold it to form a cup-like device. Physiological saline is used as the fluid to drive the device. Because the disclosed device only requires a single HFAM with small fluid volume (<1 mL) to operate, it enables driving source miniaturization to be either fully implanted under the patients' skin or attached to the body as an unobtrusive wearable device. Other than the heart, the CRD can also be used to assist other human organs that have the compression working principle such as bladder or lung.

Exo-Suit Device

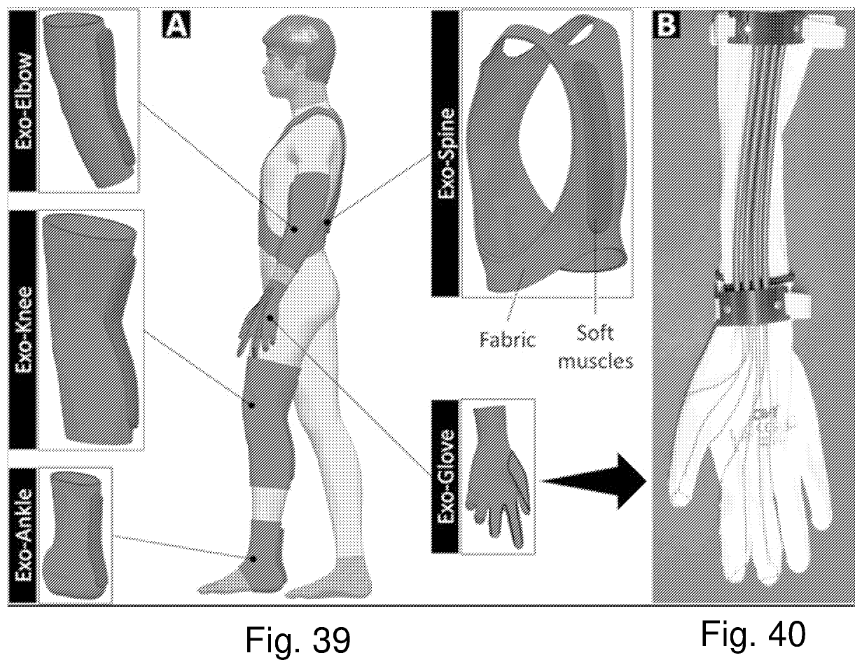

FIG. 39 shows an exo-suit made by embedding HFAMs into garments, including various configurations of an exo-suit at the desired positions: spine, elbow, knee, ankle, and glove (palm and fingers).

FIG. 40 shows a prototype of an exo-Glove.

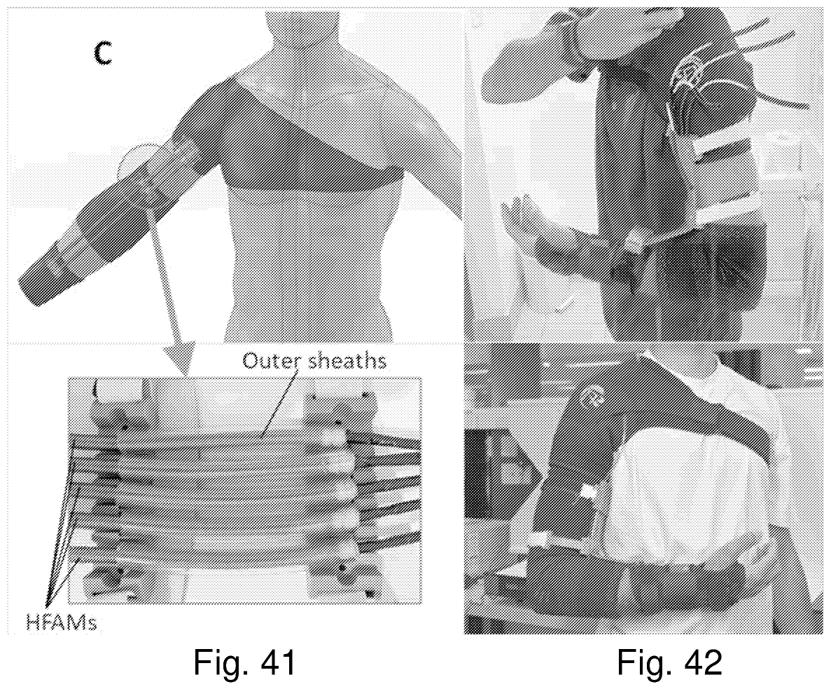

FIG. 41 shows a prototype of soft HFAM-driven wearable device-assisted upper limb.

FIG. 42 shows photographs of a final device.

Experimental Results

Both the elongation and force experiments shared almost the same components with some exceptions. An encoder and a linear slider were connected to the distal end of the muscle to collect the accumulated elongated length while a load cell was employed to record force data. The experimental platform was designed as a module system where the HFAM can be easily exchangeable. The actuation unit was equipped with a DC motor coupling with a ball screw to provide linear motion to a plunger. An encoder was placed at the motor side to track displacement of the plunger, then be converted to the input volume. The input pressure was collected by a pressure sensor that was located amid the fluid transmission tube by a T-connecter. A thin, long, and highly stretchable elastic string was used to retain the HFAM from slack while collecting elongation.

Detail Specifications of Experimental Components

| Component | Model | Specifications | Manufacturer |
|---|---|---|---|
| DC motor | 3272G024CR | Gearhead ratio: 68:1 | Faulhaber, |
| | | Encoder:IE3-1024 | Germany |
| Ball screw | SFU1204 | Shaft | XINHUANGDUO |
| | | diameter: 12 mm | AUTO, China |
| | | Lead: 4 mm | |
| Syringe | Luer-Lok ™ | Volume: 1 ml | BD Biosciences, |
| | 1 mL | Ratio: 57.3 mm/ml | Canada |
| Pressure | 40PC250G2A | Capacity: 250 psi | Honeywell, USA |
| sensor | | | |
| Encoder | S6S-1000-B | Optical, 1000 CPR | US Digital, USA |
| Load cell | LSB200 | Capacity: 1 lb | FUTEK, USA |
| Controller | QPIDe | 8 channels | Quanser, Canada |
| Force gauge | MARK-10, | Capacity: 25N | MARK-10, USA |
| | Series 5 | | |

In one embodiment, the flexible silicone microtubule can be fabricated from a rolling coating process. In another embodiment, the microtube can be manufactured by dip-coating techniques or tube extrusion method with silicone materials ranging from Ecoflex, PDMS, Exosil, Reoflex, Vytaflex, Latex rubber. The microtube can be off-the-shelf commercial medical microtube or any stretchable ones. The microtube is inserted into a constrained layer (a hollow micro-coil made from inextensible fibers or wrinkled hollow fabrics/thin tubes that can be only elongated along with its axial directions). In one embodiment, the micro-coils a type of stainless steel that can store elastic energy under the applied strain. In another embodiment, the coil can be fabricated from the fishing line, wrapped around a micro carbon fiber, following by heating in an oven to form the final coil shape. The constrained coil (inextensible fiber) can be used as a miniature extension spring like ones from MCMaster-Carr, USA, which provides a consistent diameter and axial force along the fiber length. The constrained wrinkled outer layer can be fabricated by wrinkling a hollow inextensible tube such as ultrathin-wall PET, Nylon tubes or any inextensible fabrics. In an embodiment, the outer micro-coil of the muscle can be sewing threads or fishing lines that are gently wrapped around the flexible silicone tube circumference with a rigid rode inside to prevent collapse in the silicone tube wall. To form the coil, the constrained layer is produced by wrapping the fishing line around a carbon fiber rode in a helical shape, following by heating in an oven set at 130 degrees Celsius in two hours.

Once the soft microtube is completely inserted into the constrained outer layer, its one end is tied a knot and permanently adhered into the one end of constrained layer by an adhesive glue (LOCTITE®, USA or any other adhesive glues) while the other end is connected to a commercial fluid tube (Cole-Parmer, USA or any non-stretchable and flexible microtube such as PET tubes). The fluid transmission tube is then connected to a miniature syringe via a blunt needle. To remove the air bubbles that are trapped inside the soft microtube and fluid transmission tube, the whole composites are degassed in a vacuum chamber for 30 minutes (Binder—VD115, Binder, USA) until the air bubbles are completely disappeared. When fluid pressure is applied to the actuation channel, the HFAMHFAM will be lengthened from position A at length L₀ to B at length L with a displacement x which is due to the circumferential constraint by inextensible fibers around the channels. At point B, it stores elastic energy. If a load is connected to the end of the actuator at position B and the fluid is removed, the stored elastic energy is released, allowing the actuator to apply a force against the load and bring it back to the position A. The higher pressure is applied, the higher elastic energy is stored and then a higher contraction force is achieved (at position C with length Lₘₐₓ and displacement xₘₐₓ).

In one prototype, the flexible silicone tube (microtubule) has an outer diameter of 1.1 mm, an inner diameter of 0.7 mm, and a working length of 50 mm. The HFAM is scalable and therefore its diameters and length can be varied to adapt to specific applications. The corresponding constrained coil has an outer diameter of 1.5 mm, an inner diameter of 1.1 mm that covers the entire length of the silicone microtube. In this embodiment, the use of stainless-steel coil offers many advantages including high durability and high forces compared to others such as fishing line, sewing thread or nylon coils.

In another embodiment, the actuation muscles for the devices can be obtained by twisting at least two HFAMs. This embodiment will enhance the generated force compared to a single configuration.

B. Analytical Model for the HFAM

Under some exemplary assumptions, the material volume of the silicone tube is unchanged through all stages of the HFAM structure. Using that assumption we get:

Aₜlₜ=Aᵢlᵢ=Aₚlₚ (1)

Equation (1) is then expressed as:

$$\frac{π}{4} ( {d}_{ot}^{2} - {d}_{it}^{2} ) {l}_{t} = \frac{π}{4} ( {d}_{o}^{2} - {d}_{i}^{2} ) {l}_{t} = \frac{π}{4} ( {d}_{o}^{2} - {d}_{p}^{2} ) {l}_{p} \qquad ( 2 )$$

Elongation (or strain) of the HFAM is defined by the ratio between its accumulated displacement and the initial length, ε=x/li. Letting α=lt/li is the stretching ratio of the HFAM. The value of α is obtained from experiments and is dependent on material properties and the original dimension of both helical coil and silicone tube. Equation (1) and (2) can be rewritten as follows, respectively:

αAₜ=Aᵢ=(1+ε)Aₚ (3)

α(dₒₜ²−dᵢₜ²)=dₒ²−dᵢ²=(1+ε)(dₒ²−dₚ²) (4)

Then, we can obtain the unknown inner diameters of the silicone tube when the HFAM is at the initial phase and pressurizing phase.

$${d}_{i} = \sqrt{{d}_{o}^{2} - α ( {d}_{ot}^{2} - {d}_{it}^{2} )} \qquad ( 5 )$$

$${d}_{p} = \sqrt{{d}_{o}^{2} - \frac{a}{1 + ε} ( {d}_{ot}^{2} - {d}_{it}^{2} )} \qquad ( 6 )$$

Force distribution of the HFAM can be described as:

Fₒᵤₜ=Fₜ+F_{c}−Fₚ (7)

Where Fout, Ft, Fc, Fp denote the exertion force of the muscle, the elastic force of the silicone tube, the elastic force of the helical coil, and the driving force caused by fluid pressure, respectively. The elastic force of the silicone tube is adapted from Kanno et al.'s model,

$${F}_{t} = α {EA}_{t} ( 1 - \frac{1}{1 + x / {l}_{i}} ) \qquad ( 8 )$$

Where E is Young's modulus of the silicone tube material. The elastic force of the helical coil obeys the Hooke's law that it is a product of the spring constant kc and displacement x,

F_{c}=k_{c}x (9)

The input pressure P provides the fluid force to elongate the muscle,

$${F}_{p} = \frac{π}{4} {d}_{p}^{2} P \qquad ( 10 )$$

$$= P ( \frac{π}{4} {d}_{o}^{2} - \frac{α {A}_{t}}{1 + x / {l}_{i}} ) \qquad ( 11 )$$

Exertion force of the muscle in Equation (7) can be rewritten as:

$${F}_{out} = α {EA}_{t} ( 1 - \frac{1}{1 + x / {l}_{i}} ) + {k}_{c} x - P ( \frac{π}{4} {d}_{o}^{2} - \frac{α {A}_{t}}{1 + x / {l}_{i}} ) \qquad ( 12 )$$

There are three distinct phases in the operating of the HFAM. In the initial phase, it is in a relaxed condition with no input pressure and no output force, P=0, Fout=0. In the pressurizing phase, it receives input pressure to produce elongation and accumulates elastic energy, P=P(x)>0, Fout=0. The relationship between input pressure and displacement or elongation is given in equation (13). In the releasing phase, the muscle converts elastic energy to the contraction force. The maximum output force is obtained when the pressure is completely released, P=0, Fout=Fmax(x)>0. At this phase, we can derive the relationship between output force and displacement or elongation, equation (14).

$$P = \frac{α {EA}_{t} ( 1 - \frac{1}{1 + x / {l}_{i}} ) + {k}_{c} x}{\frac{π}{4} {d}_{o}^{2} - \frac{α {A}_{t}}{1 + x / {l}_{i}}} \qquad ( 13 )$$

$${F}_{out} = α {EA}_{t} ( 1 - \frac{1}{1 + x / {l}_{i}} ) + {k}_{c} x \qquad ( 14 )$$

One of the advantages of the disclosed HFAM in some forms is that its contraction force output has the component Fc from the helical coil, which is higher than existing soft actuators with the same structure. Besides, the use of stretching ratio α also contributes to increasing the contraction force threshold due to an increase in its storage elastic energy.

Characterization and Data Analyses for the HFAM

In this section, the characterization and analysis for the HFAM in terms of its elongation and generated forces with respect to the applied fluid pressure will be carried out in the preferred embodiment with the stainless-steel coil as constrained layer and microtubule. The performance of two soft microtubule actuators connected in an axial configuration via a tactor head is evaluated. The maximization of the tactor displacement and velocity will be also introduced.

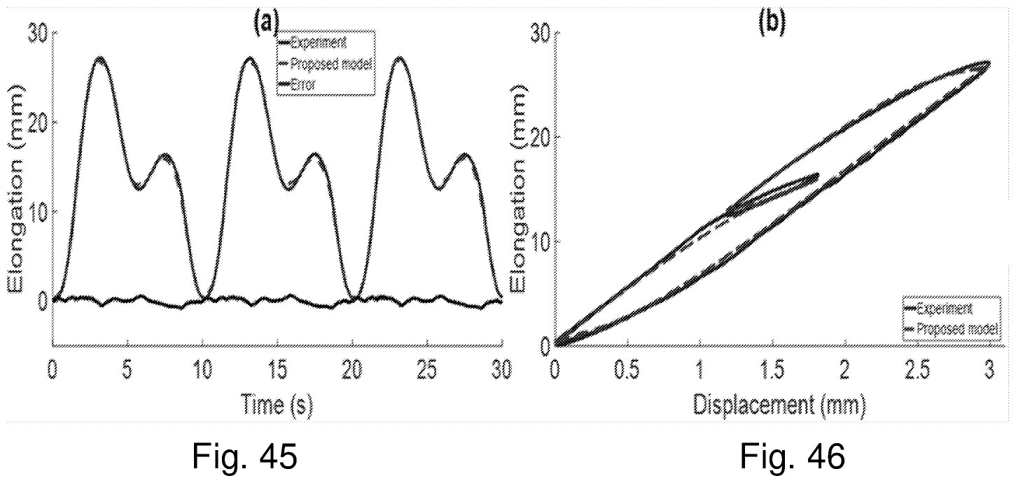

Referring to FIG. 45 to FIG. 50, an asymmetric hysteresis model is proposed for the HFAM, which offers fewer numbers of parameters compared to conventional Bouc-Wen model. In an embodiment, a simplified hysteresis shape function Ψ(x(t), \dot{x}(t), z(t)) is added to the new hysteresis shape function which can fully described by:

Φ_{S}(x,t)=αₓ₁x⁴(t)+αₓ₂x(t)+α_{z}z(t) (15)

ż(t)=|\dot{x}(t)|[Asgn(\dot{x}(t))−υ|z(t)|ⁿ⁻¹z(t)+ρ] (16)

where displacement output Φ_{S}(x,t)=xₒᵤₜ(t) and displacement input x(t)=xᵢₙ(t). This model utilizes a fourth-degree polynomial in Equation (1) for the relationship between the input and output due to the shape of hysteresis loops in experimental data. While the dimensionless parameters A, υ, ρ and n control the shape and size of the hysteresis loops, αₓ₁, αₓ₂, and α_{z} represent the ratio of output hysteresis to the input displacement and the internal state. Seven parameters are identified and optimized by minimizing the error between the modeling output and the measured output based on Particle Swarm Optimization (PSO).

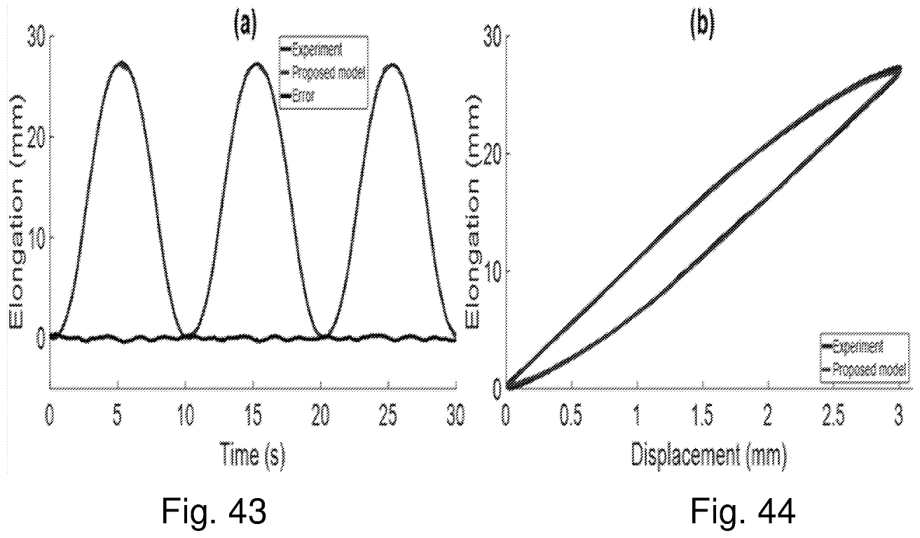

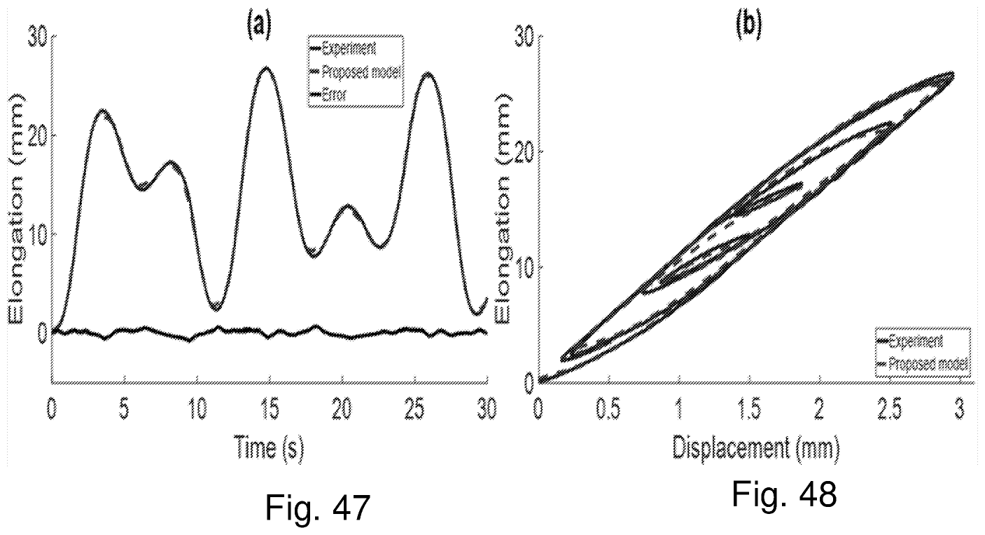

Six parameters are identified and optimized through the PSO method in Matlab with a sinusoidal input signal of 0.1 Hz. It is shown in FIG. 43 with the smallest fitness value (f=0.0215) compared to other models. The proposed hysteresis curves are asymmetric for the loading and unloading phases and it provides a good tracking performance to the experimental data. The parameters of the proposed model are identified and optimized from the PSO method that αₓ₁=−0.024, αₓ₂=10.994, α_{z}=0.272, A=−17.976, n=1.035, υ=1.563, ρ=1.391. Furthermore, FIG. 45 shows the hysteresis curves of the proposed model using the same set of parameters as revealed above and experimental results for multi-periodic inputs consisting of two signals (frequencies of 0.1 Hz and 0.2 Hz). The fitness value is f=0.1058 for this case and the results from the proposed model are close to the experimental data in the time history. Similarly, the identified results for a non-harmonic sequence are presented in FIG. 47 for the combination of 0.1 Hz and 0.1\sqrt{3} Hz with the fitness value (f=0.0769). In this embodiment, the proposed model displays a good agreement with the actual experimental data for both periodic and non-periodic input displacement of the plunger in the syringe, which drives the HFAM.

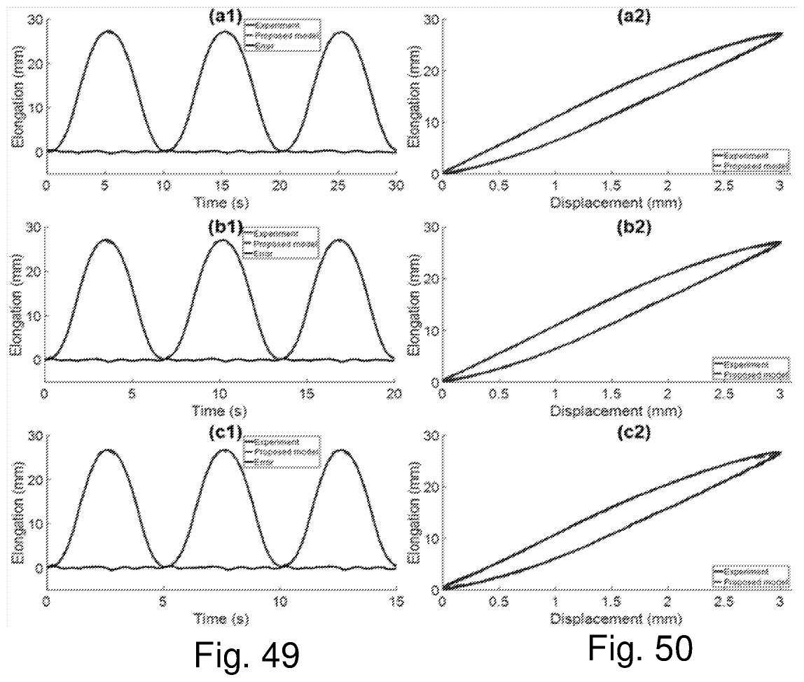

Additionally, the proposed hysteresis model is able to adapt to different velocities of HFAM elongation by testing with three sinusoidal inputs as shown in FIG. 49. They have the same amplitude of 1.5 mm and a frequency of 0.1, 0.15, and 0.2 Hz corresponding to 5.34, 8.07, and 10.75 mm/s for the HFAM average elongation velocity respectively. The same set of parameters as mentioned above is used for these cases, then the fitness function for each case is calculated with the proposed hysteresis model such as f (f_{0.1 Hz}=0.0215, f_{0.15 Hz}=0.0234, f_{0.2 Hz}=0.0389). Therefore, the proposed hysteresis model works well and is consistent in various velocities. However, the fitness function is bigger at higher velocity because the HFAM combines soft components that show non-linear characteristics with different working velocities.

Helical Gripper Embodiment

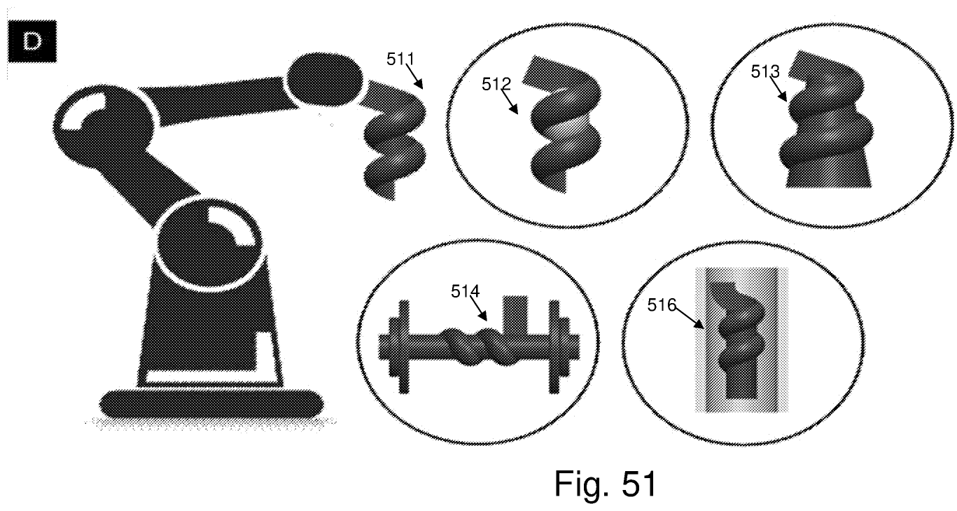

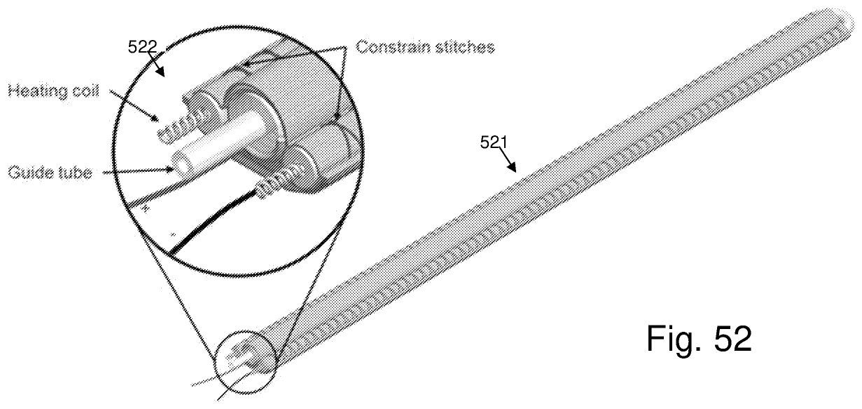

Referring to FIG. 51, there will now be disclosed a helical gripper embodiment. The helical gripper finds analogous use in nature in elephant trunks, boa constriction and cephalopod tentacles. The proposed bio-inspired soft, helical gripper with potential uses in gripping fragile objects, conforming to different shapes, lifting heavy objects and working in confined spaces. Examples are shown 511 to 516.

Referring to FIG. 52, the design and fabrication of a soft, helical gripper 521 with a variable stiffness structure (VST) and contact sensor is shown in a schematic illustration of the structure of the proposed soft gripper.

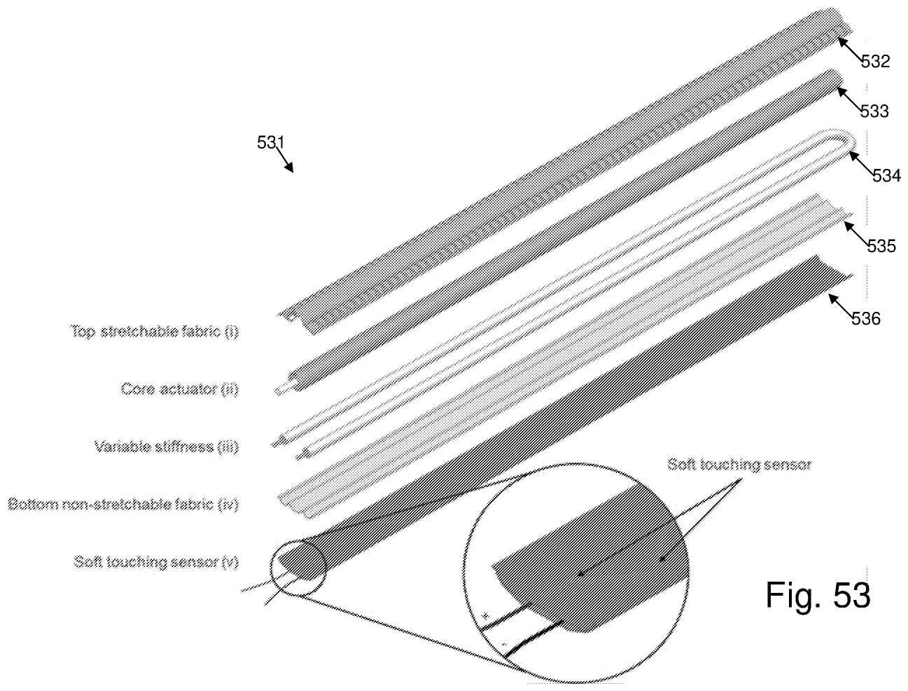

FIG. 53 illustrates the soft helical gripper in an exploded schematic form. The gripper 531 is composed of a number of main components, including top stretchable fabric 532, core actuator 533, variable stiffness element 534, bottom non stretchable fabric 535, and soft touching sensor 536. The core actuator 533 is hydraulic-driven, the fabric sleeve 532 constrains and causes the actuator to bend, a contact sensor 536 for higher sensitivity and a variable stiffness structure 5345 (VST) to enhance the load capacity.

The core actuator 533 of the soft, helical gripper is a hydraulic-driven actuator as previously described. Its construction consists of a stretchable silicone tube that is radially constrained by an inextensible coil. Both ends of the tube are securely bonded to the two ends of the coil so that both of them extend and contract together under hydraulic pressure. A non-stretchable guide tube conveys water from a syringe to the tube via a blunt needle. Upon pressurization, i.e., movement of the plunger in the syringe, the pressure inside the silicone tube increases and causes the tube to expand in all directions. However, due to the radial constraint induced by the inextensible coil, the silicone tube could only extend in one direction along the longitudinal axis, promoting the actuator to extend and twist simultaneously. The twisting direction of the actuator will be opposite to the winding direction of the coil.

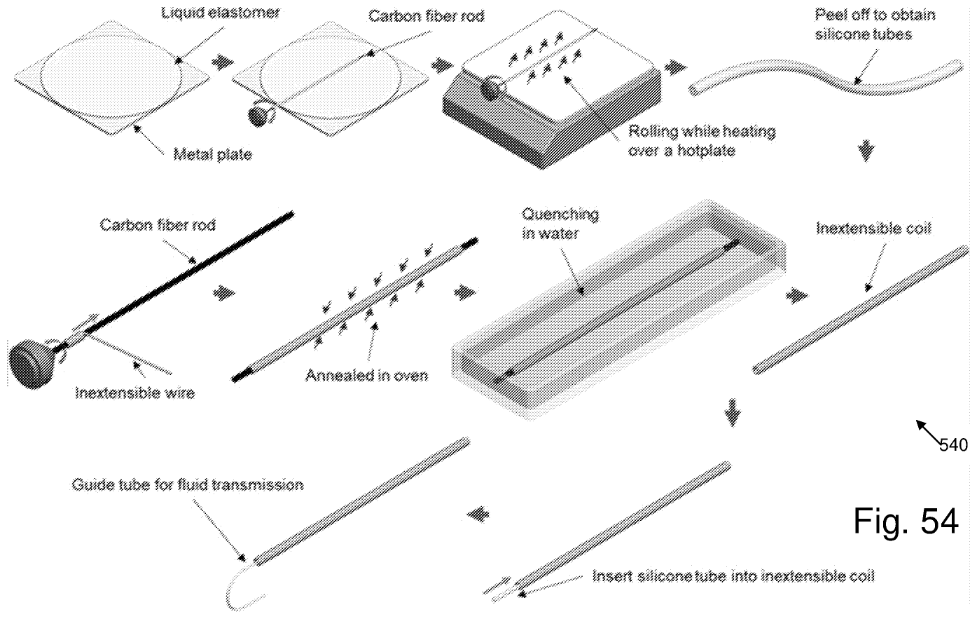

FIG. 54 illustrates the fabrication process of the core actuator 540. Briefly, the fabrication begins with the inner silicone tube by a simple roll-coating method. Platinum-cured soft elastomer (Ecoflex 00-30, Smooth-On, Inc., USA) is mixed with a weight ratio of 1:1 (part A: part B) and then spin-coated on a metal plate of desired thickness. A carbon fiber rod is then rolled onto the metal plate surface using a hand drill, and subsequently heated over a hot plate. Because the polymer has a high elongation at failure, the thin-walled tube that has been cured is then easily peeled off the carbon fiber rod. Different rod diameters can be selected in order to control the inner diameter of the silicone tube. The thickness, and hence the outer diameter of the tubes are also controllable by varying the number of rolling layers. Similarly, the inextensible coil is fabricated by winding Polyvinylidene fluoride (PVDF) fishing line around a carbon fiber rod. Coils with different inner diameters can be produced by alternating the rod diameters. The strength of the coils depends on the fishing line diameters and materials. After being wrapped around the carbon fiber rod, the coil made of the fishing line is glued at both ends and placed inside an oven at 120° C. for half an hour. Finally, the coil is quenched in water at room temperature, and the central carbon fiber is removed, resulting in an inextensible coil ready to use. The next step in the fabrication process involves inserting the silicone tube inside the inextensible coil. In order to maximize the response of the actuator, the silicone tube is fabricated to have the outer diameter being a little larger than the inner diameter of the coil. Once the tube has been inserted inside the coil, the guide tube is connected at one end of the silicone tube, and water is pumped from a syringe all the way to the other end of the silicone tube. This step is critical to ensure that all the air is removed before the tubes are sealed. Having air trapped inside the tube can adversely affect the performance of the actuator due to air compression. Inter-tubing connections are then reinforced by knots of polyester thread and superglue for better water-seal. Both ends of the silicone tube and inextensible coil are then bonded together by superglue so that the actuator could extend and twist due to the constraint of the inextensible coil.

While the core actuator provides extension and twisting motions, the fabric sleeve constrains the actuator to bend and, therefore, wind up into the helical shape. The fabric sleeve is fabricated by combining fabrics and stitches in a multilayer structure with conduits for actuator insertion. There is a wide variety of options that fabrics and stitches can be combined to create the fabric sleeve for the helical gripper.

In order to cause bending, the fabric sleeve should be able to extend on one side while the other side is strain limited. Non-stretchable fabrics, such as cotton weaves, should be a suitable material for this purpose while the extending side should be made of either non-stretchable fabrics with wrinkles or stretchable fabrics. Stretchable fabrics can be either uniaxial elastic or biaxial elastic. These fabrics are usually made of elastic fibers, such as Spandex, spun into stretchable yarn and joined along weft, warp, or both directions of the weave, yielding uniaxially or biaxially stretchable fabrics, respectively.

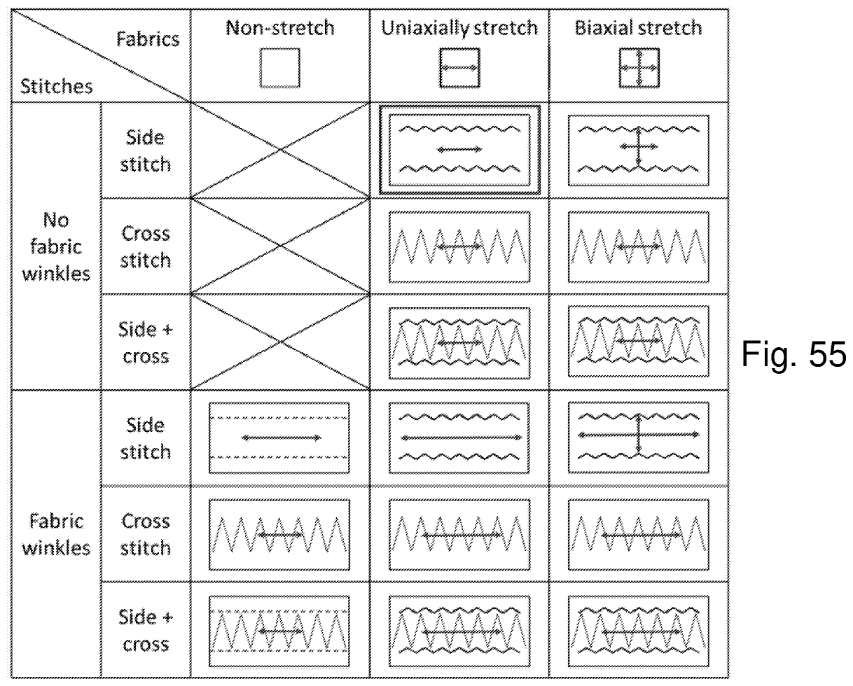

Stitching is necessary to join layers of fabrics and form fabric conduits for tube insertion. Different stitch designs can yield distinct patterns of stretchability in the assemble gripper. Three different combinations of fabric types and stitch designs with their corresponding stretchability are displayed in FIG. 55.

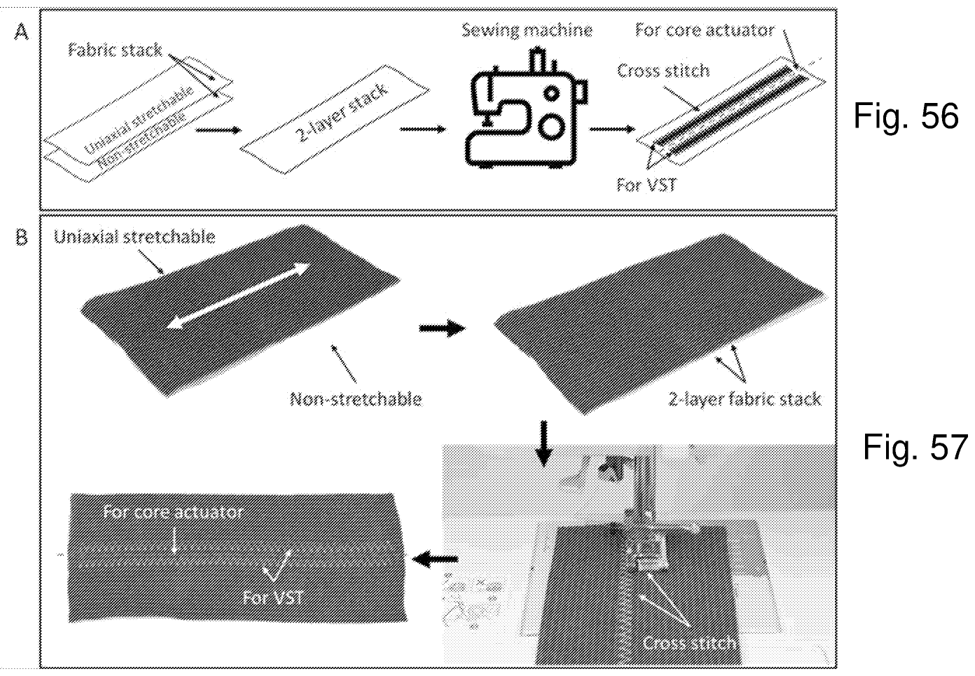

From the information provided in FIG. 55, in order to achieve an optimal combination between simple fabrication and performance, in this work, in some forms, the system requires the use of a piece of uniaxial stretchable fabric for the top layer and another piece of non-stretchable fabric at the bottom. One form of fabrication process is illustrated schematically in FIG. 56, and in further detail in FIG. 57.

In the first step, the fabric layers were aligned and stacked. Cross-stitch was then used to form the channels while preserving the stretchability of the top layer. In addition, the use of cross-stitch helps save fabrication time by allowing more channels to be formed with fewer numbers of sewing times, i.e., three channels could be formed with two stitches. The stitching patterns can be designed to realize different configurations for a single tube or multiple tubes, depending on applications. In the end, a fabric sleeve is provided with three channels, including a large channel in the center for insertion of the core actuator and two smaller channels running alongside the central channel that are for insertion of the variable stiffness structure (VST).

Different sewing methods can be used to form the fabric sleeve, including hand sewing, machine sewing and computerized embroidery. When the pattern size fits within machine limits, computerized embroidery is preferred because of its automation, great accuracy, flexibility and efficiency. When embroidery is not available, machine sewing is the second-best option. This method involves manual movement of the fabric under the sewing presser foot. Hand sewing is the least efficient method, nevertheless, it can help accommodate complex stitch paths or non-flat surfaces which are involved when turning stitches are necessary.

The variable stiffness structure in this work is made of PET medical tubes (ID: 0.89 mm and OD: 2.16 mm) inserted with a stainless-steel coil sheath (Asahi Intecc, Japan) (OD=0.84 mm) as the heating source. PET was selected among other thermoplastic materials due to its immense stiffness change between glassy and rubbery states, biocompatibility, high strength, relatively low glass transition temperature (around 67° C.), high chemical resistance, and low cost. Because of these benefits, PET was already reported as a potential variable stiffness manipulator for surgical robots by Huu Minh Le et al. The selected stainless-steel coil sheath used in this paper is highly flexible and, therefore, contributes little effect on the stiffness of the gripper in the rubbery state. There are several other advantages of using the proposed variable stiffness structure design. Firstly, using the coil sheath as the heating source can speed up the phase transition because, with the same overall length, a coil sheath can generate more heat and more even heat distribution than a single wire. Secondly, with the tubular configuration, the inner channel of the variable stiffness structure can be utilized for other purposes such as an active cooling system to reduce the cooling time of the gripper, which has long been a challenge hindering thermally-activated variable stiffness structures from practical applications. In the proposed design of the helical gripper, the variable stiffness structure is inserted inside fabric channels, running side-by-side with the core actuator. When a current is passed through the heating coil, the coil will heat up due to Joule-heating effect and soften the PET tubes, allowing the soft helical gripper to freely deform. When the current is removed, PET tubes will cool down and stiffen, holding the gripper at the current shape. Unlike the conventional designs which usually heat up the entire gripper due to the larger area covered by the variable stiffness layer, this design will have a benefit that the heat will be accumulated in a much smaller area, along the variable structures and therefore, will consume less input energy and affect less to other parts of the gripper such as the contact sensor. This design also allows the VST to be facilely attached or removed from the gripper according to applications. For example, the VST can be used when high-load holding is required while it is not necessary for grasping lightweight objects.

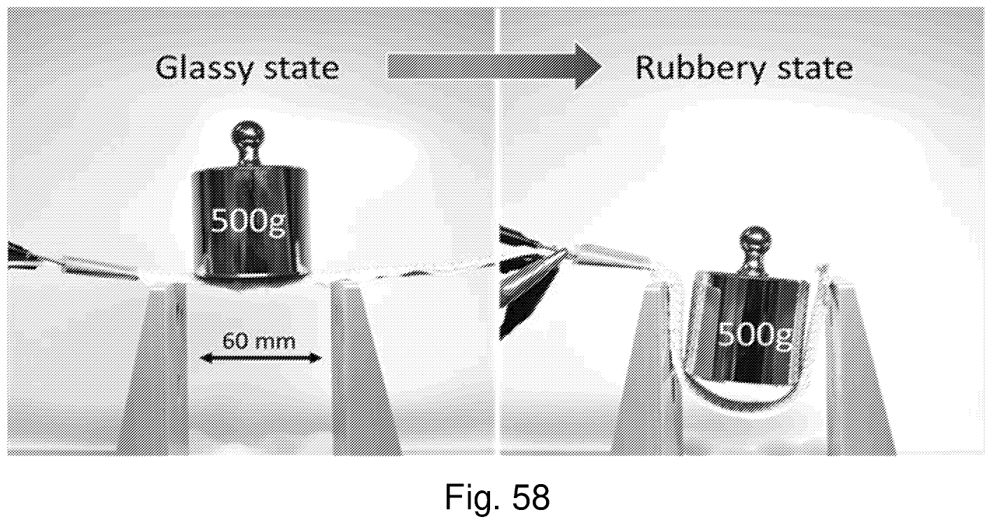

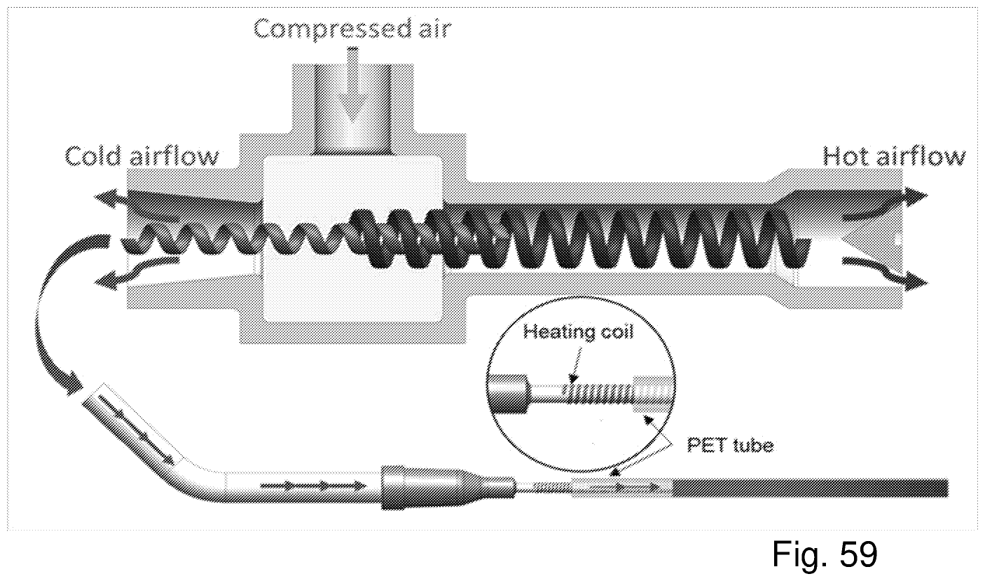

In order to accelerate the transition of the VST from the rubbery state to the glassy state, a compact-sized, low-cost and widely available vortex tube is used to blow a cold air stream through the inner hollow channels of PET tubes. In FIG. 58, the phase transition from the glassy state to the rubbery state of the variable stiffness structure is demonstrated. At the glassy state, the structure is stiff enough to support a 500 g weight while at rubbery state, it becomes soft, and under the load of the weight, the structure deforms, which leads to a large bending. FIG. 59 describes the working principle of the vortex tube to create a cold air stream and the design of the VST to accelerate the cooling time.

Returning to FIG. 53, a soft, stretchable contact sensor 536 based on eutectic gallium-indium (EGaIn) is arranged along and covers the non-stretchable side of the helical gripper to provide sensory feedback of touching when the actuator winds around the objects. The sensor is a wavy-shaped and single-pixel pressure sensor of which the resistance changes under external pressure due to dimensional changes of the EGaIn channels. However, unlike other approaches reported in the literature that used the EGaIn channels embedded inside a block of elastomer, we first fabricate electroconductive microtubules containing EGaIn and then wrapped them into the wavy patterns (the pitch between each turn is kept at 8 mm). A thin layer of silicone glue is used to keep the conductive microtubules in shape, leaving most of the microtubule body unconstrained and free to deform. This design allows higher sensitivity of the sensor thanks to a greater extent of deformation of the EGaIn channels compared to conventional sensors that have a bulk of silicone surrounding the channels, resulting in more constraint and less deformation.

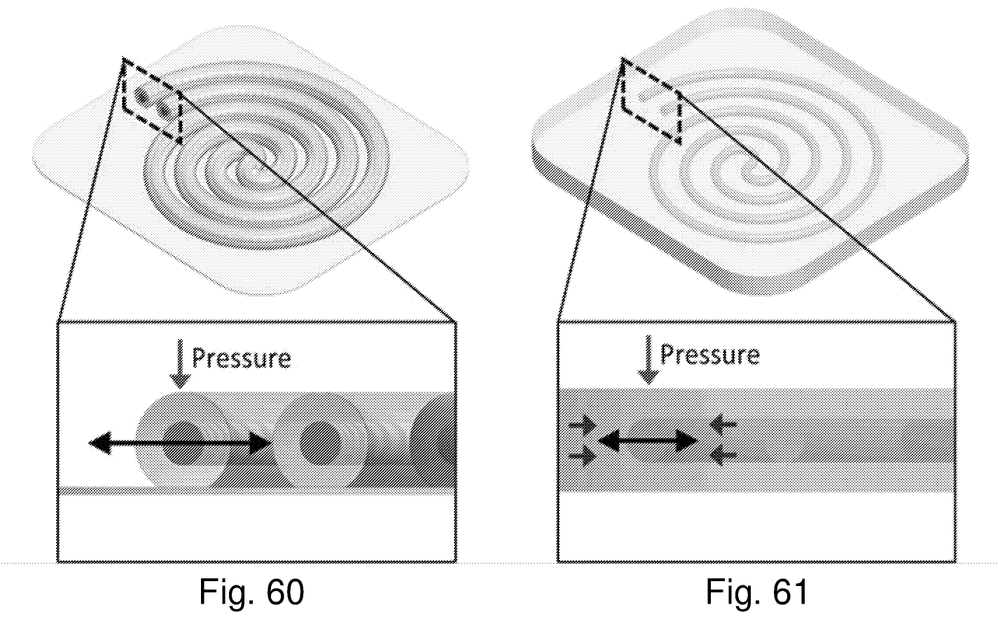

Turning to FIG. 60 and FIG. 61, for a better illustration purpose, the two sensors in FIG. 60 are patterned in spiral shape instead of wavy shape that will be used with the actual helical gripper.

Without wishing to be bound by theory, a simplified model to describe the deformation of the helical gripper was developed. The model was conducted with the assumption that the cylindrical geometry of the silicone tube maintains for both the undeformed and deformed configurations and that the fluid pressure is uniformly distributed inside the silicone tube. We also assume that the silicone tube behaves as a Neo-Hookean solid and that the strain energy per unit volume of the actuator u can be expressed as a function of the first invariant of the Cauchy-Green strain tensor I₁ as follows:

I₁=λ₁²+λ₂²+λ₃² (17)

u=C₁(I₁−3) (18)

where C₁ is the experimentally determined material constant and λ₁, λ₂, and λ₃ are the Cauchy-Green strains which define the deformation ratio in the length, the circumference and the thickness of the silicone tube, respectively. The incompressibility of the silicone tube implies that:

λ₁λ₂λ₃=1 (19)

Under pressurization, the core actuator undergoes a coupled extension-rotation deformation with ω representing the rotation of the actuator about its central axis. The inextensibility of the inextensible coil induces a constraint on the silicone tube deformation that can be expressed as:

$${λ}_{1}^{2} {cos}^{2} α + {λ}_{2}^{2} {sin}^{2} {α ( \frac{γ + ω}{γ} )}^{2} = 1 \qquad ( 20 )$$

$$γ = \frac{l}{{r}_{o}} tan α \qquad ( 21 )$$

where α is the helix angle of the inextensible coil, and γ is the number of turns of the inextensible coil expressed in radian (rad). (γ/2π defines the number of turns of the inextensible coil).

The internal volume of the actuator, V, and the volume occupied by the silicone tube, Vt, can be expressed by:

V=πλ₁l(rₒ−λ₃(rₒ−rᵢ))² (22)

Vₜ=πl(rₒ²−rᵢ²) (23)

where rᵢ, rₒ are the inner and outer radii of the silicone tube and l is the initial length of the actuator. During the working period, due to the constraint of the inextensible coil, the strain in circumferential direction was negligible so we can assume that λ₂=1. With this assumption and equation (3), if λ₁=λ, then we can have:

λ₃=1/λ (24)

This condition correctly describes the deformation of the silicone tube that under the fluid pressure, if the tube length increases, the thickness of the silicone tube will be reduced accordingly. Substituting for λ₁, λ₂, and λ₃, equations (4) and (6) can be rewritten in terms of extension ratio λ. From the virtual work presented by Trivedi et al, we can relate the change in total strain energy of the actuator to the change in the work done by the pressure P as:

$$P \frac{dV}{d λ} = {V}_{t} \frac{du}{d λ} \qquad ( 25 )$$

where dV and du are the virtual changes in the internal volume of the actuator and strain energy stored in the silicone tube in terms of the virtual change dλ. For any given pressure value, we can solve for the corresponding extension ratio λ by inverting the equation (24). From equation (20), we can further express the rotation of the actuator, ω, upon pressurization in terms of λ as:

$$ω = \frac{l}{{r}_{o}} ( tan α - \frac{\sqrt{1 - {λ}^{2} {cos}^{2} α}}{cos α} ) \qquad ( 26 )$$

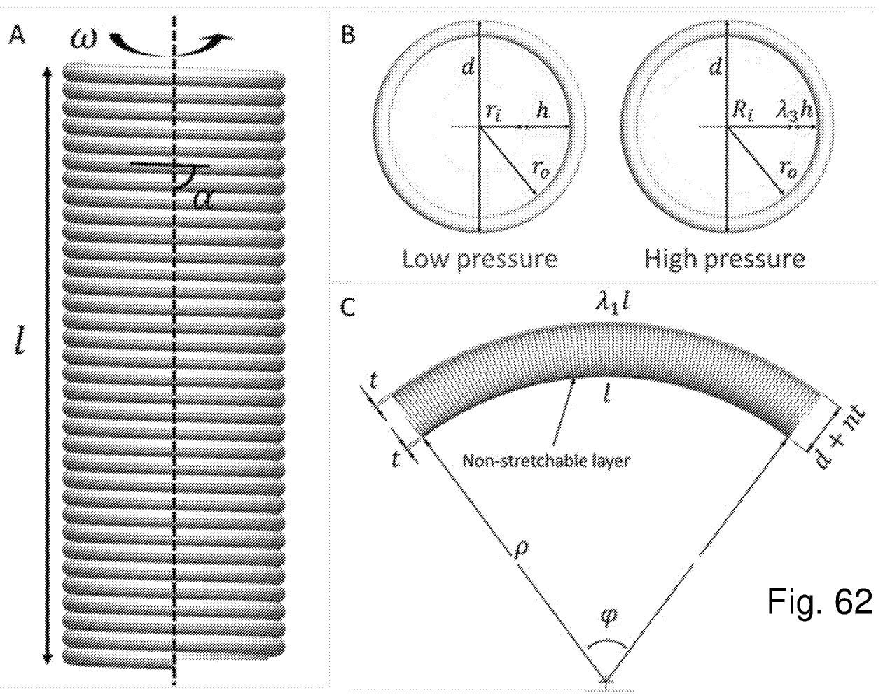

From FIG. 62, we can derive the helix radius of curvature, ρ, and therefore the curvature, κ, and the torsion, τ, of the helical actuator from the extension ratio λ and rotational angle ω, as follows:

$$ρ = \frac{d + n t}{( λ - 1 )} \qquad ( 27 )$$

$$κ = \frac{1}{ρ} = \frac{( λ - 1 )}{d + n t} \qquad ( 28 )$$

$$τ = \frac{ω}{l} \qquad ( 29 )$$

where n is the number of fabric layers, t is the thickness of each fabric layer, assuming all the layers have the same thickness and d is the diameter of the actuator, respectively. The radius of the helix can then be calculated by:

$${R}_{helix} = \frac{κ}{{κ}^{2} + {τ}^{2}} \qquad ( 30 )$$

We performed characterizations of the continuum, soft helical gripper fabricated by experimentally studying the relationship between input pressure and corresponding deformation of the gripper, thermal properties of the proposed variable stiffness structure, response of the integrated contact sensor under external pressure and realizing the helical gripper prototypes in different scenarios, grasping a variety of objects with different shapes and sizes. Dimensions of the helical gripper prototypes and their components are summarized in Table 1 below and remain unchanged throughout implemented experiments.

TABLE 1
Dimensions of the helical gripper and its
components used in characterization.

| | |
|---|---|
| Length (mm) | 130 |
| Width (mm) | 16 |
| Thickness (mm) | 3.6 |
| PET tubes (ID × OD × length) (mm) | 0.89 × 2.16 × 140 |
| Silicone tube (ID × OD) (mm) | 1.5 × 3 |
| Inextensible coil (ID × OD) (mm) | 2.35 × 3.15 |
| Weight (with VST) | 8.2 g |

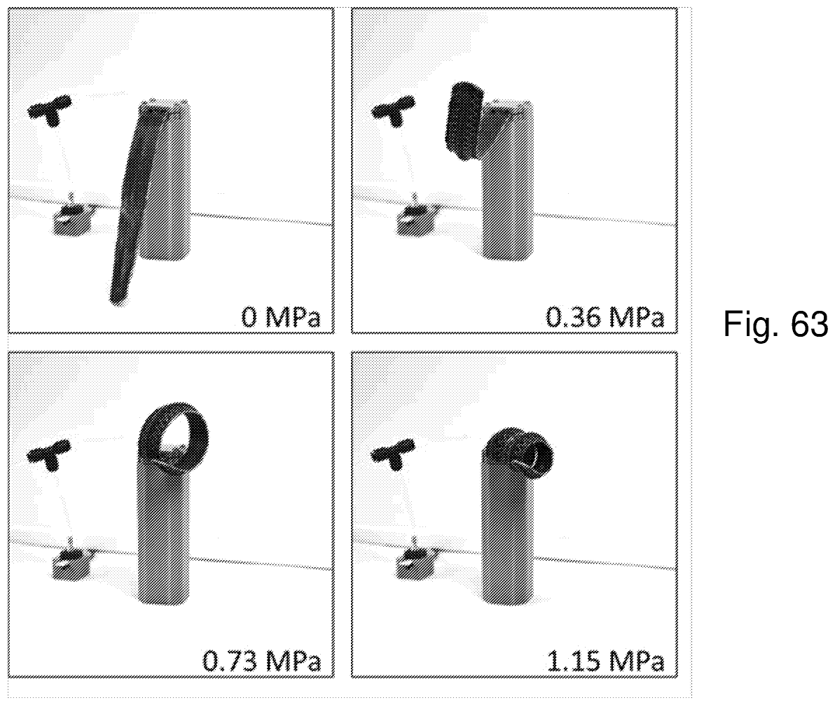

The behavior of the soft, helical gripper is experimentally characterized under various input pressure values. A syringe was used to pump water through the guide tube into the core actuator, causing the pressure to increase. Upon pressurization, the core actuator, being constrained radially by the inextensible coil, starts to elongate and twist simultaneously. However, the presence of the fabric sleeve introduces an anisotropic restriction on the elongation of the actuator, giving rise to bending. Both motions work together and cause the soft gripper to bend and twist while elongating, resulting in the helical shape. The more pressure is applied to the gripper, the more it will wind up and reduce the diameter. FIG. 63 displays the range of deformation of the soft, helical gripper corresponding to different input pressure values.

The heating and cooling behaviors of the designed variable stiffness structure (VST) were studied. In order to realize fast heating to the VST, we studied the heating rate of the VST by heating time while using different current values. The heating power follows the Joule-heating formula P=I².R, where I is the current passing the heating coil, and R is the resistance of the coil. The VST was tested with five values of electric current, ranging from 0.1 A to 0.5 A. A thermal camera (FLIR Inc., USA) was used to measure the change in temperature at the middle point of the continuum gripper upon heating while another DLSR (Nikon Inc., Japan) camera recorded the heating process in videos for later analysis.

The PET tubes that are used for the VST had the glass transition temperature starting at about 65° C. to 70° C., and entered a rubbery state at about 80° C. with its stiffness being significantly decreased. On the opposite side, the PET tubes recover most of their stiffness at about 50° C. and slightly stiffen when the temperature gets lower. Considering these thermo-mechanical properties of PET tubes, in this paper, the heating time would be defined by the interval for the VST to reach 80° C. from 25° C. while the cooling phase of the VST was ceased when the temperature reduced to 50° C. As anticipated, when the current is increased, the time required to heat up the VST decreased. For example, it takes less than 10 s and 15 s to heat the VST to 80° C. with 0.5 A and 0.4 A, respectively.

In order to accelerate the cooling rate, a design employing a vortex tube was used to create a cold air stream at 13° C. from 500 KPa compressed air and blow it through the hollow channels of PET tubes. Cooling time by vortex mechanism was recorded at two locations along the VST, including middle point and tip point, and compared with values by ambient cooling. We hypothesize that the further it is away from the air source, the longer it will take to cool down, and therefore, the tip of the VST will take a little more time to cool down than the middle point.

It was found that ambient cooling required about triple the time to cool down below 50° C. compared to vortex cooling, and there was no significant distinction in the cooling rate between the middle and tip point. On the contrary, as anticipated, vortex cooling could efficiently speed up the cooling rate by reducing the cooling time at the middle point to 8 s and at the tip point to 11 s to drop the temperature below 50° C.

Compared to previously reported active cooling systems that used either water or air, the proposed design using the vortex tube is among those that achieve the highest cooling rates. Moreover, the design has a simpler construction than those systems that employ water for cooling due to no requirement for circulating systems, rendering it more practical for applications. In this work, the soft, helical gripper could complete a thermal cycle in approximately 24 s, considering the heating of 0.5 A and vortex cooling time at the tip point. This value is a little longer than the sum of heating and cooling time reported above due to the temperature overshoot over 80° C.

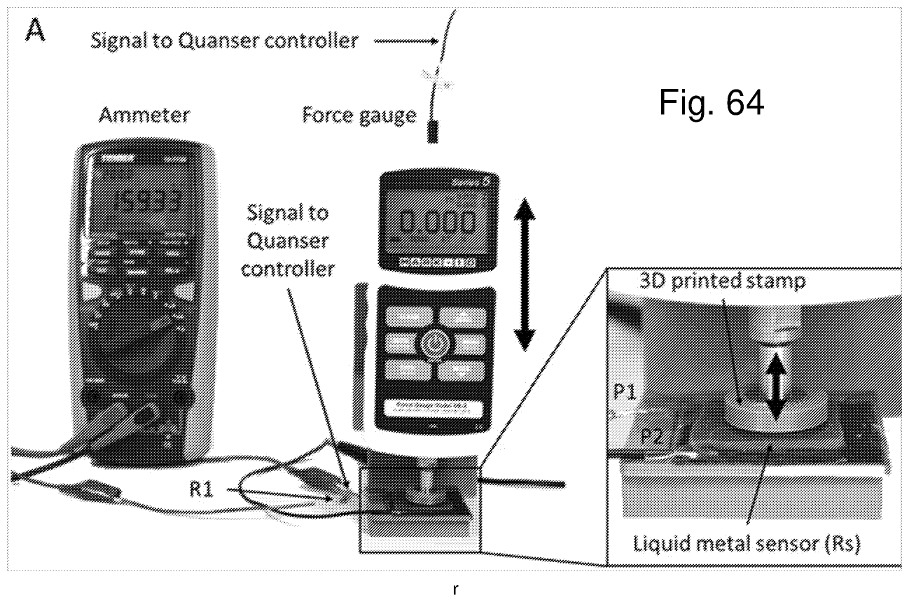

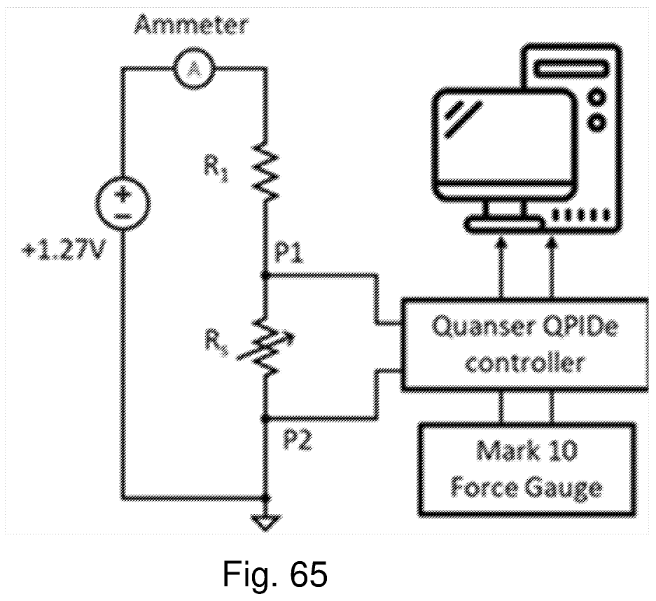

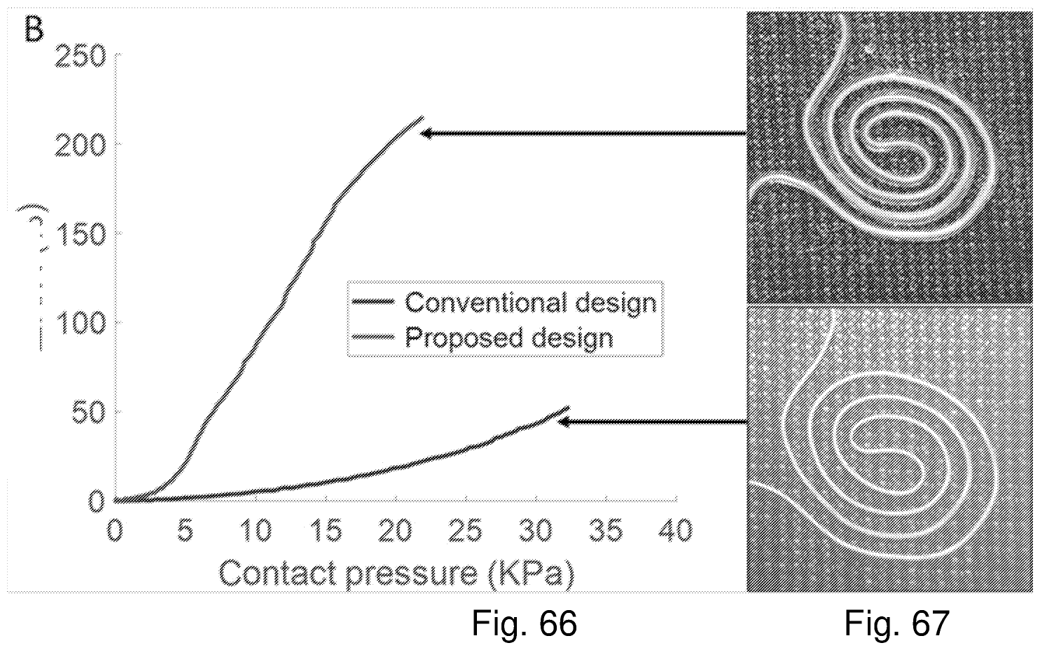

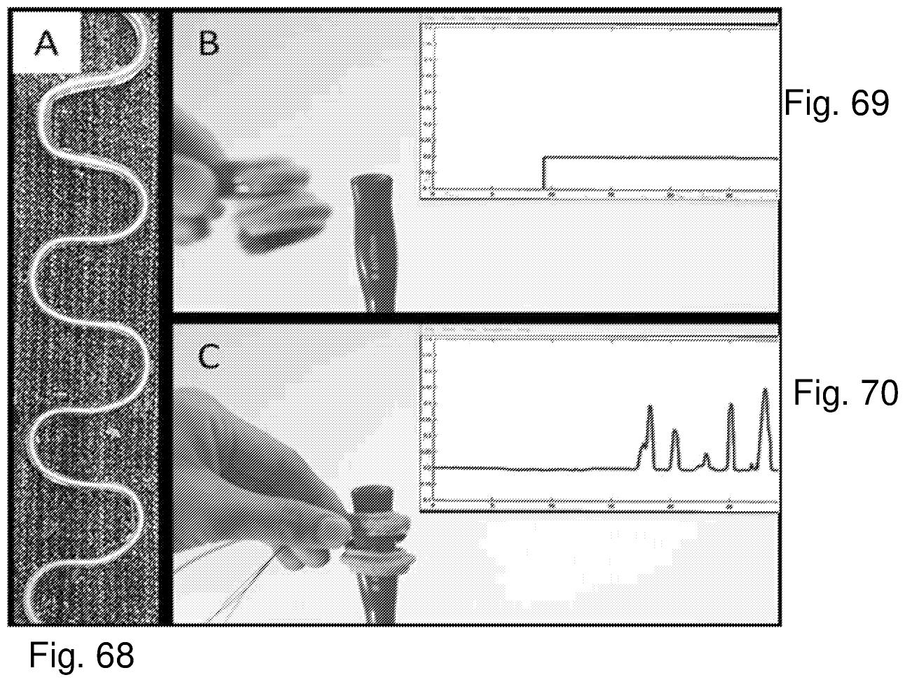

In order to demonstrate the enhancement in sensitivity of the sensor design, two soft, EGaIn-based sensors were fabricated and characterized by their change in resistance under the normal contact pressure, as displayed in FIG. 64. Both sensors were single-pixel and followed the spiral shape. At first, one sensor was fabricated by elastomeric microtubules filled with EGaIn and arranged in the spiral pattern on a piece of fabric. It was then fixed to a 3D printed base by a piece a double-sided tape and placed underneath a Mark-10 force gauge. A 3D printed circular stamp was attached to the force gauge shaft to cover over the entire surface area of the sensor to be tested. In order to read signals from the sensor, a simple voltage divider circuit (as shown in the schematic diagram in FIG. 65) was employed. The change in sensor resistance, ΔR, was derived from the change in voltage across the sensor (P1 and P2 in the schematic diagram). A vertical translation stage with micrometer steps was employed to press the force gauge against the contact sensor. Both the voltage data from the sensor and the force data from the Mark-10 force gauge were collected by the QPIDe controller board (Quanser) and transferred to a PC. The other sensor was fabricated by covering the first sensor with more elastomer, resulting in liquid metal channels embedded in a block of elastomer as conventional sensor designs. This sensor also underwent the same experiment as the first one, and their ΔR/R under normal force are compared in FIG. 66 for the two designs shown in FIG. 67 and FIG. 68.

As FIG. 66 indicates, the change in resistance, ΔR/R, significantly increases with the normal force applied directly upon the contact sensor. As hypothesized, the sensitivity of the sensor with the design has been noticeably improved compared to the other one. At 15 KPa, while ΔR/R of the conventional design was only 10.6%, that value of the novel design reached up to 157%, approximately 15 times larger than the former one. However, similar to previous observations reported for liquid-metal-based soft sensors, the ΔR/R of both sensors here was found to be nonlinearly related to the contact force.

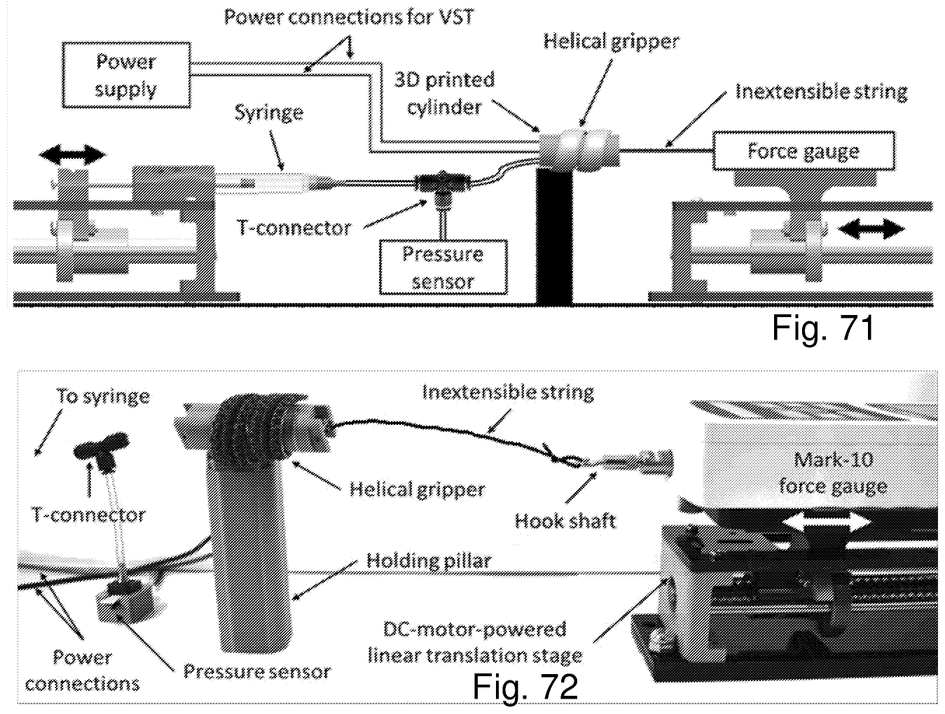

For the dynamic response test, instead of the spiral shape that was used in the previous experiment, a wavy-shaped sensor, as displayed in FIG. 69, was integrated into the gripper, covering along the non-stretchable surface. The reason for this change was because the spiral shape is more suitable for the pressing experiment, in which the stamp could press on the whole sensor, generating the highest sensitivity. On the other hand, since the helical gripper is long and thin and it winds its body around the objects during gripping, a wavy-shaped sensor that covers along the entire gripper surface will also ensure the same effect. Measurements were then recorded under two scenarios: when the gripper grasped with no load (we hypothesized there would be no significant change in ΔR/R) and when the gripper grasped an object (a screwdriver in this case and we hypothesized of significant change in ΔR/R). Multiple inflation-deflation cycles were conducted for each scenario. As hypothesized, under freeload condition, no significant change in ΔR/R was observed while changes in ΔR/R corresponding to each time the gripper grasped the screwdriver was clearly visible. FIG. 70 and FIG. 71, shows the changes in resistance of the contact sensor under two gripping scenarios.

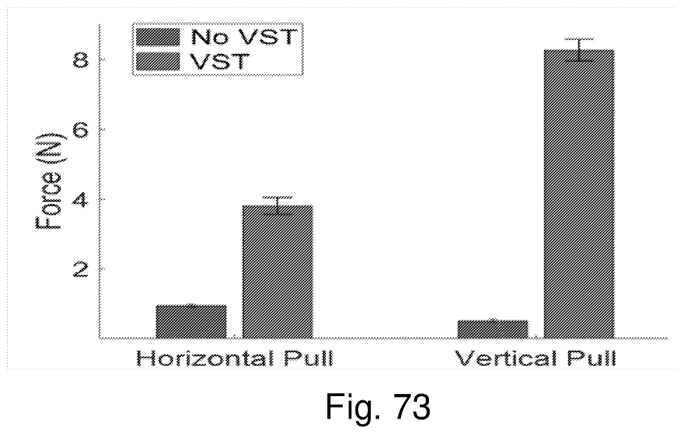

As shown in FIG. 72, in order to realize the improvement in load capacity of the integration of the VST into the helical gripper, a holding force test was conducted. In this experiment, a soft helical gripper with the dimensions listed in Table 1 above was used to helically grip a 3D printed cylinder with a diameter of 20 mm. The gripper was pressurized until 1.15 MPa so that it could grasp two full turns around the cylinder. The gripper was then anchored to an optical table (Thorlabs Inc., USA) while the cylinder was connected to the Mark-10 force gauge by an inextensible string. A DC-motor-powered linear translation stage moved the Mark-10 force gauge and pulled the cylinder from the gripper. Since helical gripper can approach objects from multiple directions, two gripping configurations were chosen to be investigated in this experiment, including horizontal pulling (the pulling direction was along the longitudinal axis of the cylinder) and vertical pulling (the pulling direction was perpendicular to the lateral surface of the cylinder). Both grippers with VST and without VST were tested on each configuration for five times, and the peak force of each combination was calculated. It was hypothesized that the helical gripper would have a stronger holding force in vertical pulling than horizontal pulling. Therefore, the force gauge displacement was 30 mm in horizontal pulling tests, while the displacement was 40 mm in vertical pulling tests. The force was defined as the peak pulling force in the process of moving the force gauge on the linear translation stage, starting from the position where the display of the force gauge was not zero. FIG. 72 illustrates the experimental setup for the horizontal pulling test of the helical gripper with VST, while FIG. 73 displays the real setup. (The helical gripper without VST underwent the same experiment, except the use of power supply for heating VST).

As anticipated, under both horizontal and vertical pulling tests, the helical gripper without VST exhibited low holding force with the peak forces were approximately 0.9N and 0.5N for horizontal and vertical pulling, respectively. These results did not agree with the hypothesis about vertical pulling having greater holding force than horizontal pulling. This may be attributed to the force direction that caused the helical gripper to uncoil during vertical pulling, resulting in much lower holding force than that of horizontal pulling, which did not experience uncoiling. In addition, significant deformations to the gripper shape were visible in both pulling configurations. This was also the reason contributing to the low holding force of the helical gripper without VST. On the contrary, as shown in FIG. 36, with the integration of the VST, the peak holding forces were increased in both pulling configurations. The peak holding forces were approximately 3.8N and 8N for horizontal and vertical pulling, respectively, which accounted for four times and 16 times greater than the results of gripper without VST. There was also no significant deformation to the gripper shape observed in this case. It is also noted that both grippers with and without VST could fully or partially restore their initial gripping states when the pulling force was released (the force gauge was translated backward) except for horizontal pulling of the gripper with VST in which the cylinders already slipped out of the gripper's grasp. The results from this experiment confirmed that the VST could not only enhance the load capacity of the soft, helical gripper but also help maintain the gripper shape under loading, preventing the gripper from being damaged.

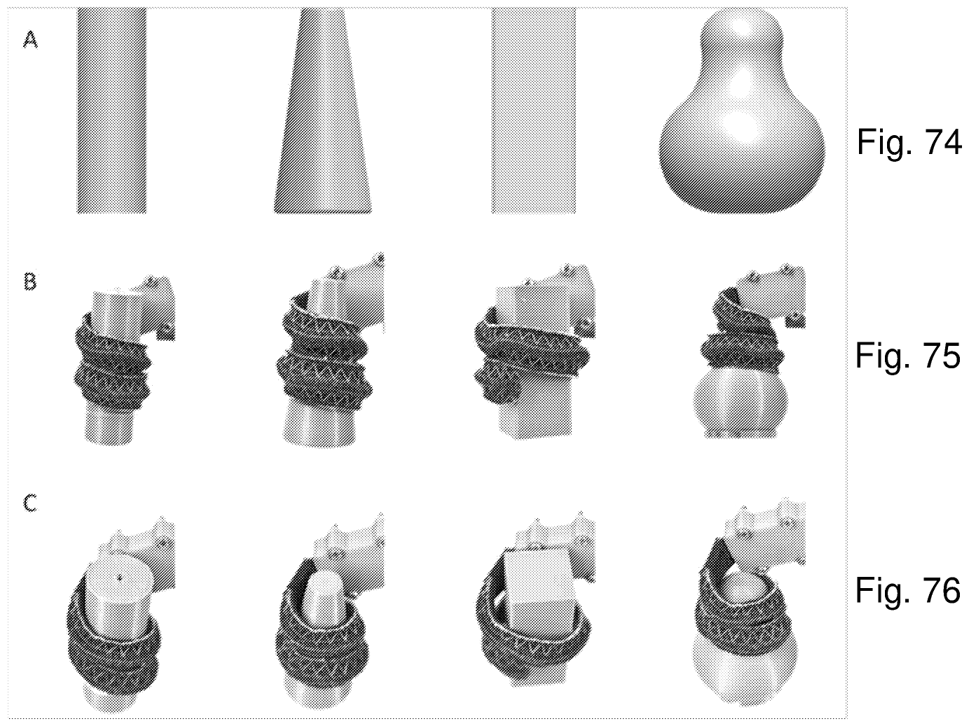

Turning now to FIG. 74, continuum soft grippers following the helical configuration offer the advantage of high conformability to objects of various shapes, sizes and poses. In order to illustrate this, experiments were conducted below involving grasping objects of four basic shapes, including cylinder, rectangular prism, cone and gourd. These shapes were selected from others because they are the basic shapes that appear in almost every object in real life. Four objects with the desired shapes were designed, and then 3D printed using an Ultimaker 2+3D printer. FIG. 74 shows experiment four objects that were used in the conformability while FIG. 75 and FIG. 76 display the conformability of the helical gripper when grasping these objects from the front and top views, respectively. Dimensions of the helical gripper used in the experiment were kept as listed in Table 1 above. Because the weight of these objects was light, gripper without VST was used in this experiment to mitigate complexity in control. The images show that the helical gripper was able to conformally wrap around three other objects, while the rectangular prism was the only one that could not be conformally grasped by the gripper. This result can be attributed to the sharp edges of the rectangular prism that prevented the gripper from squeezing inward, leaving a small gap between the gripper and the prism surfaces. (FIG. 37C—3rd image). Nevertheless, the gripper was able to firmly hold the rectangular prism object, even under vigorous touching and being thrown at by another object.

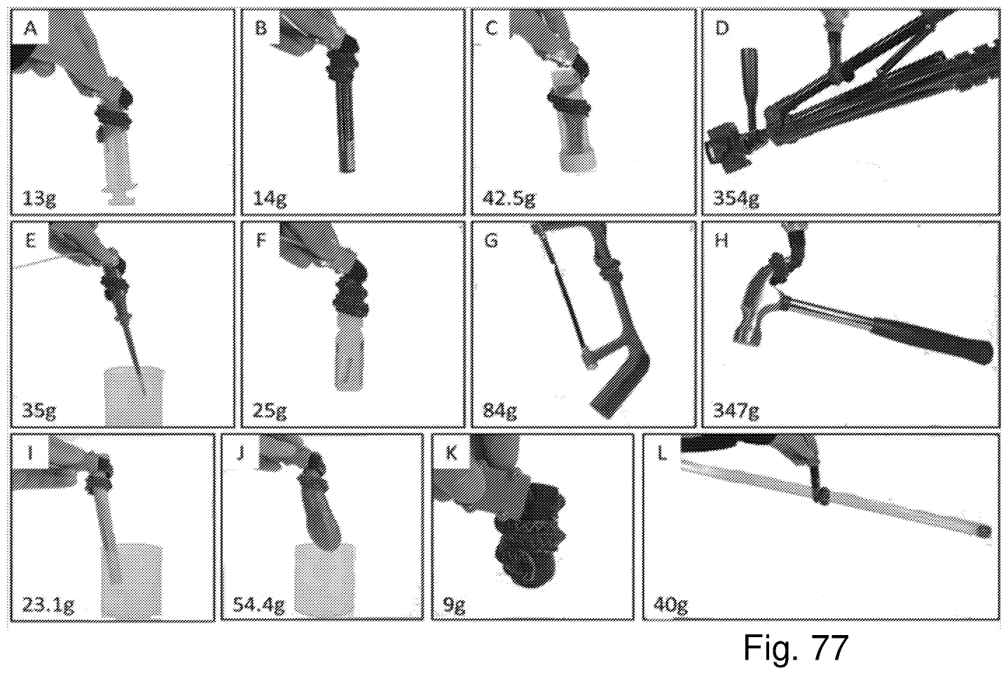

FIG. 77 demonstrates the helical gripper gripping a variety of objects of different geometries and weights. In some forms the disclosed helical gripper may facilitate a safe grasp of fragile objects as well, thanks to the buckling effect of the core actuator in this design. When inflated, the gripper works by extending and coiling around the object. In no object condition, increasing pressure causes the gripper to increase the number turns and reduce the inner diameter of the helical shape. However, when the gripper reaches the size of the object, the helical diameter cannot become smaller, causing the core actuator to buckle inside the fabric conduit, and the squeezing action stops. The buckling effect prevents the gripper from damaging fragile objects, as shown in FIG. 77 I, J, K, even under the application of large input pressure. FIG. 77 L shows that the helical gripper is an ideal option for grasping long and slender objects that are challenging for multi-fingered- and closed-grippers.

Since the continuum, helical gripper is long, flat and thin, it is especially suitable for gripping tasks in confined environments where fingered, or close-structured grippers cannot fit, or their sizes should be significantly reduced, which subsequentially diminishes their performance.

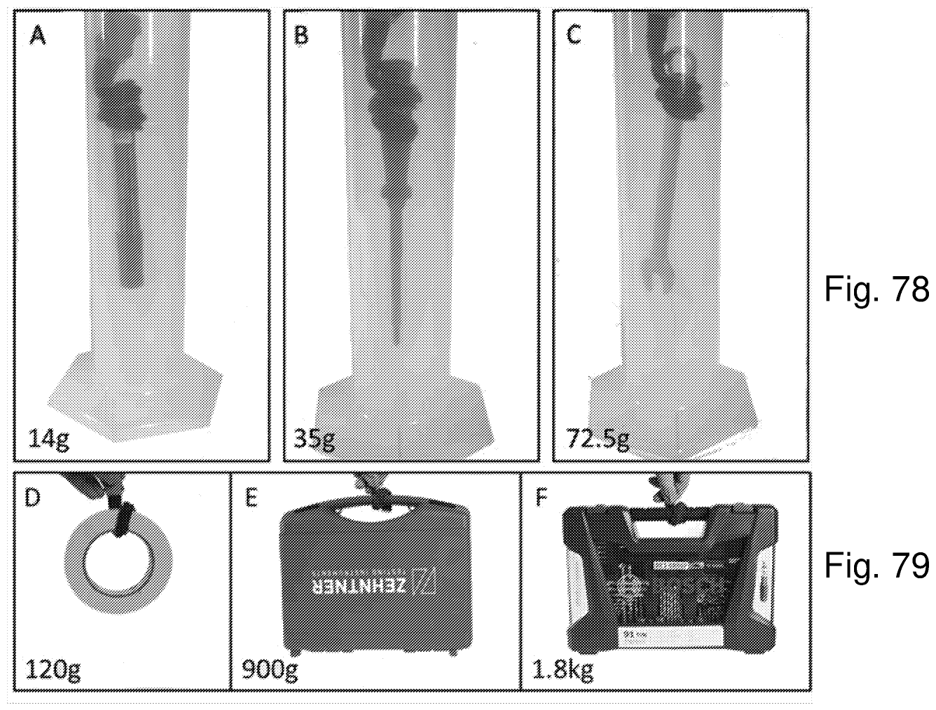

An experiment was conducted to demonstrate the ability of the continuum, helical gripper to go inside a cylindrical tube of 62 mm diameter and 410 mm length and retrieve objects such as a marker, a screwdriver and a wrench as shown in FIG. 78A, B, C. The helical gripper could successfully wrap around the object bodies and retrieve them from the tube. This capability of the helical gripper would be useful for exploration, rescuing and manipulation applications in confined and hazardous environments.

In addition, thanks to small footprint and continuum structure, the helical gripper may utilize slots, holes or handles that are available on object bodies to grasp and lift objects. This is the feature that is not present in many grippers nowadays. Being able to utilize slots, holes or handles available on objects, the helical gripper offers the ability to grip much larger and heavier objects compared to the conventional way by enclosing objects from outside. FIG. 79 is an example of the helical gripper hooking through the center hole and lifting a roll of masking tape. When combined with the variable stiffness, this feature further enables the helical gripper to grip and lift much heavier and larger objects such as the Zehntner film applicator case in FIG. 79 (900 g) and a Bosch tool case in FIG. 79 (1.8 kg). These two objects are approximately 110 times and 220 times heavier than the mass of the gripper, respectively.

HFAM-Driven Smart Surgical Sutures

A further application of the HFAM device will now be described.

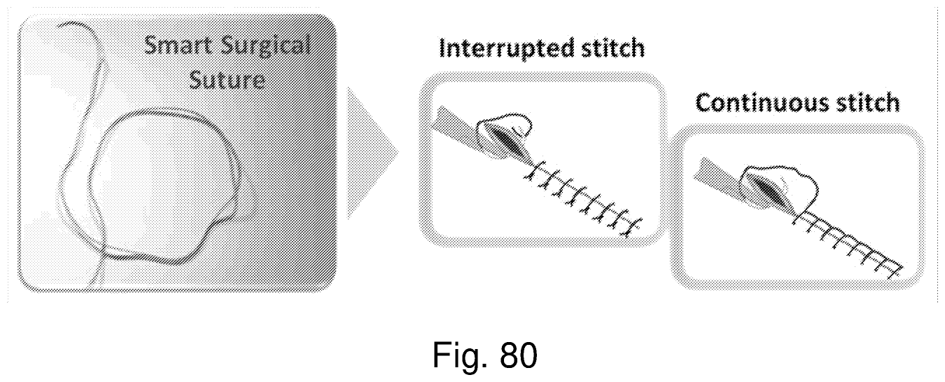

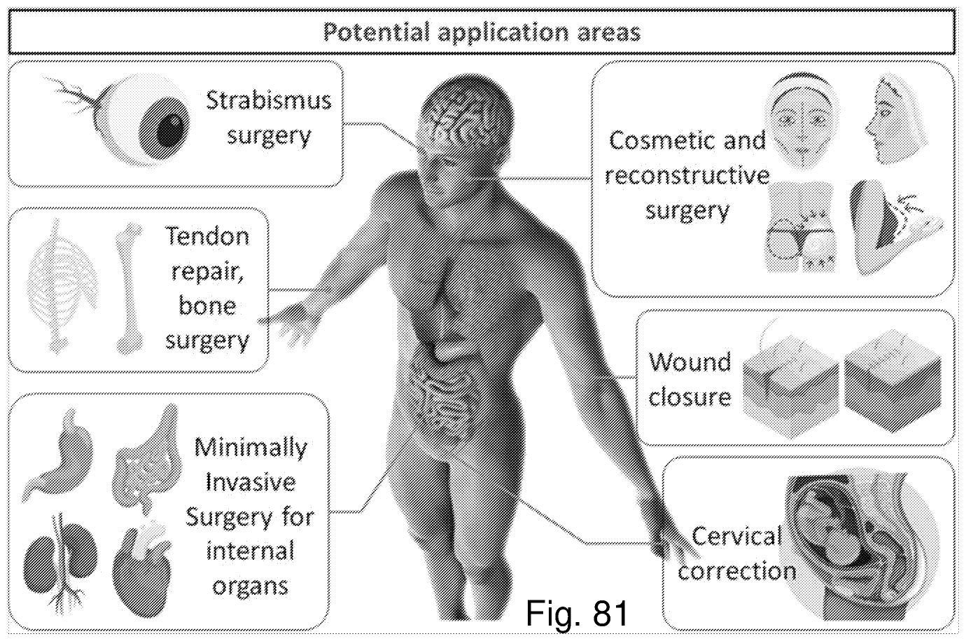

FIG. 80 illustrates the smart surgical sutures (S2 sutures), which are able to provide interrupted and continuous stitch. FIG. 81 illustrates their potential application areas.

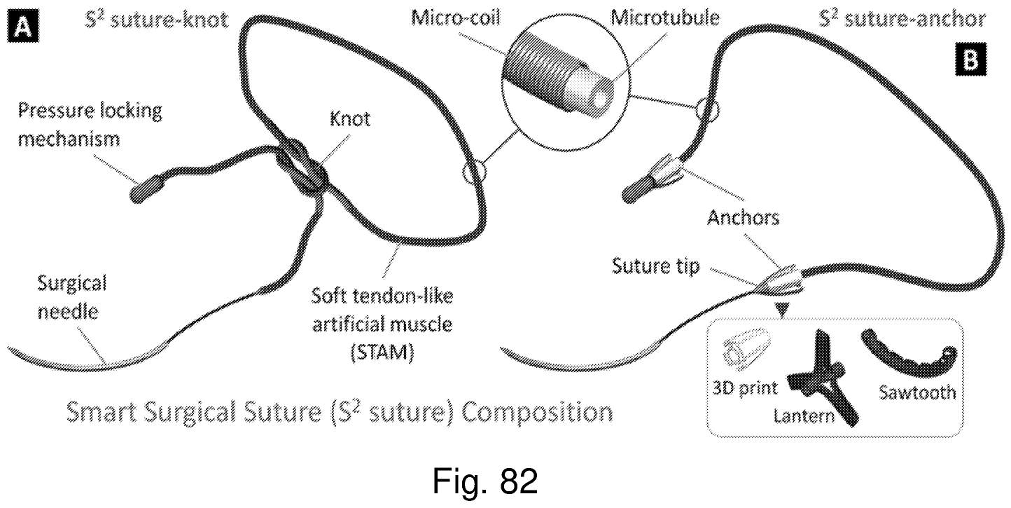

Turning now to FIG. 82, there is provided more details of the S2 suture composition. Wound closure with surgical sutures is a critical challenge for flexible endoscopic surgeries. The HFAM can be used to develop functional and smart surgical sutures (S2 sutures) to either monitor wound conditions or ease the complexity of knot tying. The S2 sutures not only offer effective anchoring functions of both knot tying and barbed anchors but also provide desired and uniform tensile distribution without the need for additional intervention. For ease of comparison, the S2 suture with knotting function is named “S2 suture-knot” while that with barbed anchors is named “S2 suture-anchor.” Typically, the S2 suture-knot consists of a soft tendon-like artificial muscle or HFAM, a pressure locking mechanism (PLM), and a commercial surgical needle (FIG. 82). The HFAM is a flexible, soft artificial muscle made from a miniature soft silicone tube inserted into a micro-coil so that it can be elongated to store elastic energy upon hydraulic pressurization and exert contraction force when releasing the pressure. Regarding the S2 suture-anchor composition, both ends of the HFAM are equipped with locking anchors (FIG. 82). These anchors can be automatically deployed to secure the tissue without the need for a surgical knot which requires complex manipulation of the closure device. One end of the HFAM is connected to a pressure locking mechanism to hold and release its pressure. The other end of the HFAM is connected to a cone-shaped suture tip and a curved surgical needle to facilitate the tissue puncture.

Both the S2 suture-knot and S2 suture-anchor are equipped with a pressure locking mechanism (PLM) to hold the inner pressure of the HFAM at a predetermined threshold to maintain the desired elongation. After making all stitches, the PLM can be cut to release the pressure to shorten the HFAM length. When producing a HFAM, a flexible tube called fluid transmission tube is used to connect the HFAM body to a fluid source. While input pressure from the fluid source to the HFAM is maintaining, the fluid transmission tube is locked and becomes a PLM.

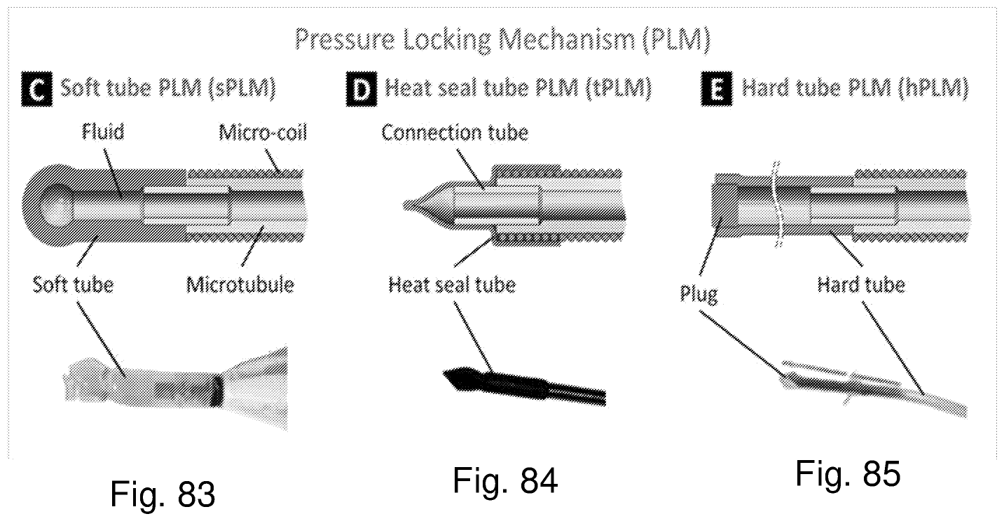

Turning to FIG. 83 to FIG. 85, three different PLM designs including a soft tube PLM (sPLM, FIG. 83) made from soft rubber tubing, a heat seal tube PLM (tPLM, FIG. 84) made from flexible polyethylene terephthalate (PET) tubing, and a hard tube PLM (hPLM, FIG. 85) made from polytetrafluoroethylene (PTFE) tubing. Three types of PLMs require different locking methods: a simple overhand knot for the sPLM, heat seal effect and reinforced thread for the tPLM, a cylindrical plug for the hPLM. The sPLM is easier to cut but has a relatively lower pressure threshold and thus smaller suture tension compared to the tPLM and hPLM.

The anchors are responsible for holding separated tissues in place so that surgical knots can be eliminated, releasing surgeons from performing the toughest tasks, especially in confined spaces during endoscopic surgeries.

FIG. 82 illustrates three different designs for the anchors: 3D print, lantern, and sawtooth. The 3D printed anchors are made from hard plastic materials by commercial 3D printers. They have a cone shape with 4 barbs to facilitate tissue puncture in one way and locking the suture in the opposite direction. The lantern and sawtooth anchors are flexible plastic hollow tubes with patterned cuts so that they can be deployed to hold the separated tissues once the fluid pressure is released. A tube with longitudinal cuts (spare at two ends) produces a lantern-like shape when lengthwise compressing its two ends. The triangle cuts (sawtooth) create a bending anchor upon deployment where the HFAM is shortened after hydraulic depressurization.

The S2 suture can adjust its length to achieve the desired tension at the time it is fabricated and automatically tighten its knot or deploy anchors to stabilize the suture against the sewed tissues without using any external pulling force, which is normally required in conventional sutures. In addition, the S2 sutures are also equipped with different anchors that can effectively secure the defect tissues without using knots, offering flexible choices to meet different demands of wound closure. The S2 sutures can be used in wound closure and tissue folding for applications of strabismus surgery, tendon/bone repair, minimally invasive surgery for internal organs, cosmetic and reconstructive surgery, cervical correction, and other related wound closure procedures (Fig. S1-S6).

FIG. 82 illustrates structure of the smart surgical sutures (S2 sutures), including the S2 suture-knot can be knotted as conventional surgical sutures and the S2 suture-anchor formed by combining the S2 FIGS. 83 to 85 illustrated three different types of anchors illustrating design of different pressure locking mechanisms (PLMs) and their prototypes.

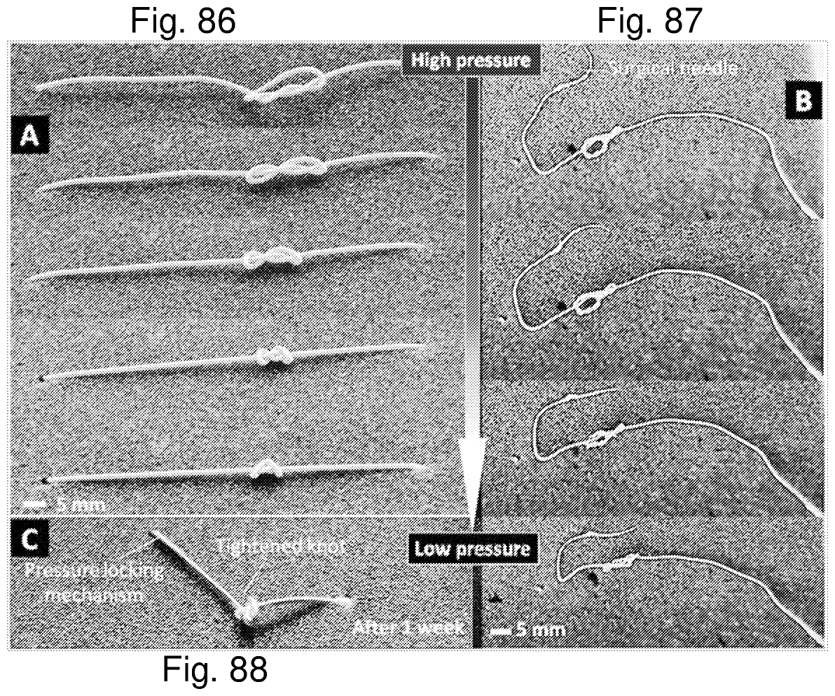

FIG. 86 illustrates the self-tightening capability and knot security of the S2 suture-knot. In A, a prototype (OD1.49×L70 mm) is pressurized to 100% elongation and tied a loose knot with both ends are fixed. The knot is tightened when reducing input pressure. FIG. 87 illustrates a similar method to FIG. 86 but with a prototype OD0.8×L100 mm and both ends are set free. FIG. 88 illustrates stability of the tightened knots after one week.

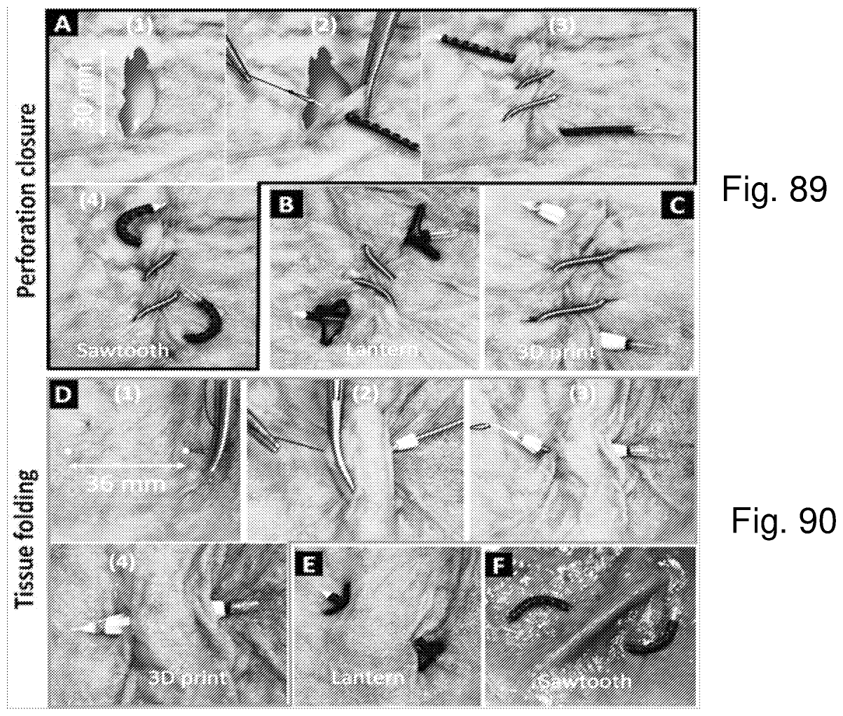

FIG. 89 illustrates perforation closure with the S2 suture-anchors. A) Perforation closure procedure with 6 running stitches by sawtooth anchor suture. B, C) The same procedure applies to the lantern and 3D printed anchor sutures, respectively.

FIG. 90 illustrates the tissue folding procedure (weight loss surgery) by 3D printed anchor suture. E, F) The same procedure applies to the lantern and sawtooth anchor sutures, respectively.

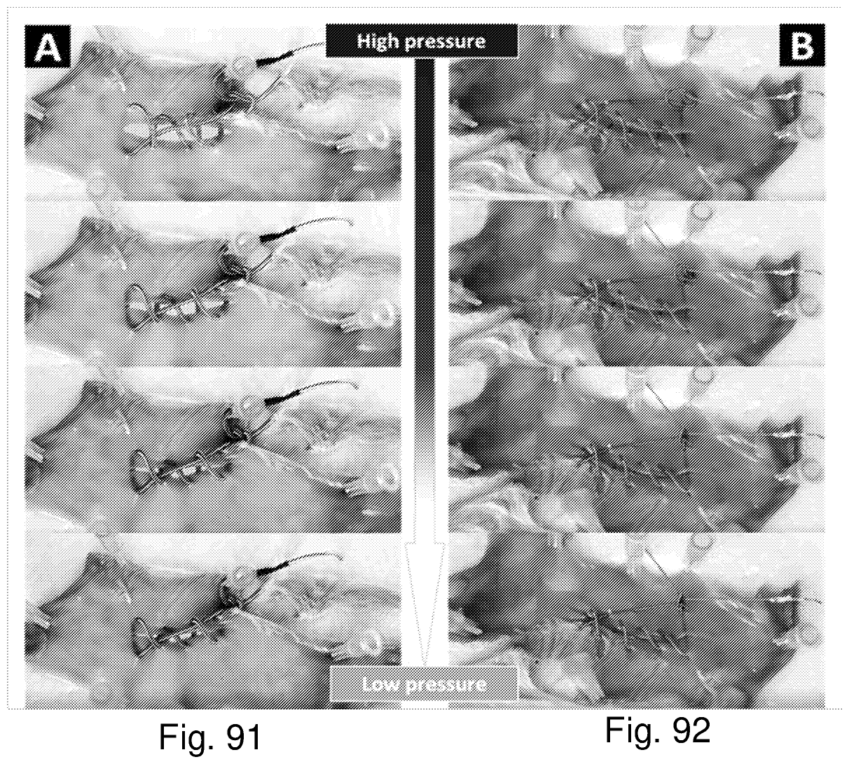

FIG. 91 illustrates perforation closure with the S2 suture-knots on a fresh porcine colon, with Results for the S2 suture-knot OD1.49×L70 mm FIG. 92 illustrates B, results for the S2 suture-knot OD0.8×L100 mm.

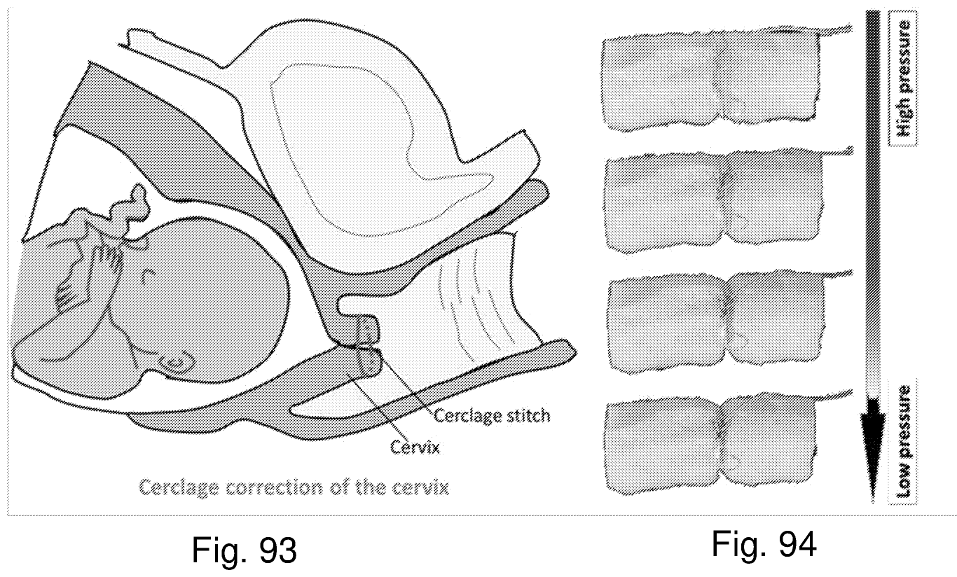

FIG. 93 illustrates the cerclage correction of the cervix with the S2 suture-knot. FIG. 94 illustrates a prototype which is pressurized to 60% elongation, wrapped around a soft foam, tied a knot, and finally released the fluid pressure to self-tighten and secure the knot.

While the technology has been described in reference to its preferred embodiments, it is to be understood that the words which have been used are words of description rather than limitation and that changes may be made without departing from its scope as defined by the appended claims.

### INTERPRETATION

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1-3. (canceled)

4. A method of fabricating a soft filament artificial muscle comprising inserting a microtubule into an outer coil, the microtubule having first and second ends.

5. The method as defined in claim 4, further comprising tying off the first end of the microtubule and engaging it with the coil.

6. The method as defined in claim 4, further comprising attaching the second end of the microtubule to a fluid source.

7-16. (canceled)

17. A gripper system comprising a soft fabric gripper having a continuum helical shape.

18. A gripper system as defined in claim 17, the soft fabric gripper comprising a core actuator that is hydraulic-driven

19. The gripper system as defined in claim 18, the soft fabric gripper comprising a fabric sleeve that constrains and causes the core actuator to bend.

20. The gripper system as defined in claim 17, further comprising a contact sensor.

21. The gripper system as defined in claim 17, further comprising a variable stiffness structure to enhance the load capacity.

22. An elongated actuator comprising:
an elongated inner tube for carrying a pressurized actuation fluid;
a helical coil wrapped around the elongated inner tube;
wherein the actuator undergoes actuation by means of pressure fluctuations in the elongated inner tube.

23. The elongated actuator as claimed in claim 22, wherein the inner tube is open at at least one end and attached to a fluid pressure control means for causing controlled pressure fluctuations in the inner tube.

24. The elongated actuator as claimed in claim 22, wherein said helical coil formed from one of metal wire, fishing line, a polymer or sowing thread.

25. The elongated actuator as claimed in claim 22, wherein said actuator is twisted, knitted, weaved, or braided to form a fabrics or rope structure.

26. A collection of elongated actuators as claimed in claim 22 attached to at least one substrate so as to cause relative controlled movement thereto.

27. The elongated actuator as claimed in claim 22, wherein said actuator tube expands on pressure increase and contracts on pressure decrease.

28-30. (canceled)

31. A helical gripper comprising an actuator as claimed in claim 22.

32. The elongated actuator as claimed in claim 22, wherein said fluid is actively cooled.

33. (canceled)

34. The elongated actuator as claimed in claim 22, the elongated actuator being configured to be twisted, knitted, weaved, or embedded to create smart garments or active objects.

35. The elongated actuator as claimed in claim 22, the elongated actuator being arranged in a spiral arrangement such that the spiral arrangement expands radially when the pressure within the elongated actuator increases.
